(19) United States
(12) Patent Application Publication
(10) Pub. No.: US 2023/0313099 A1
(43) Pub. Date: Oct. 5, 2023

(54) MULTI-WELL DEVICE, KITS AND METHODS FOR ANALYSIS OF CELLS

(71) Applicants: Subin Mac GEORGE, Saint-Laurent (CA); Margaret Haiganouch MAGDESIAN, Saint-Laurent (CA); Xue Ying CHUA, Montreal (CA); 9493662 CANADA INC., Montreal (CA)

(72) Inventors: Subin Mac GEORGE, Saint-Laurent (CA); Margaret Haiganouch MAGDESIAN, Saint-Laurent (CA); Xue Ying CHUA, Montreal (CA)

(73) Assignee: ANANDA DEVICES INC., Montreal, QC (CA)

(21) Appl. No.: 18/043,931

(22) PCT Filed: Sep. 3, 2021
(86) PCT No.: PCT/CA2021/051226

§ 371 Date: Mar. 3, 2023

Related U.S. Application Data
(60) Provisional application No. 63/209,561, filed on Jun. 11, 2021.
(60) Provisional application No. 63/074,628, filed on Sep. 4, 2020.

(51) Int. Cl. C12M 1/32; C12M 3/06; B01L 3/00; C12M 1/00

(52) U.S. Cl. CPC C12M 23/12; B01L 3/502707; C12M 23/16; C12M 23/22; B01L 2300/0829

(57) ABSTRACT

Systems and methods for culturing and/or analyzing cells are provided. The system can include a microfluidic layer, a multi-well grid layer and a base layer. An electrode layer can optionally be provided. The system can also include an alignment feature for aligning microchannels of the microfluidic layer with electrodes of the electrode layer to achieve a predetermined organized architecture of the microchannels relative to the electrodes. The system can include a plurality of microfluidic layers and a microfluidic layer engaging frame engageable with the plurality of microfluidic layers to form a unitary structure engageable with a multi-well plate comprising a plurality of wells each comprising an electrode layer. A well identification feature associated with a microfluidic unit can be provided on an upper surface of the multi-grid layer to enable visual identification of a well of the multi-well grid layer that is in fluid communication with a microfluidic unit.

### CROSS REFERENCE TO RELATED APPLICATIONS

The present application claims priority from U.S. Provisional Pat. Applications No. 63/074,628, filed on Sep. 4, 2020, and No. 63/209,561, filed on Jun. 11, 2021, the disclosures of which are hereby incorporated by reference in their entirety.

### TECHNICAL FIELD

The technical field generally relates to cell analysis and more particularly to multi-well devices, kits and methods for high throughput and high content analysis of cells in a microfluidic environment.

### BACKGROUND

Current efficacy and toxicity evaluation of chemical and biological compounds in contact with humans such as medications, cosmetics, pesticides, pollution, etc., relies on massive screening of compound libraries against various extra- and intracellular molecular targets to find compounds with the desired mode of action. While high-throughput technologies for chemical synthesis and automation technologies for isolation of natural products have significantly increased in the past years, there is still no efficient method to evaluate the effects of these large collections of compounds in a wide range of cellular assays.

In neurobiology research and drug discovery, High Throughput Screening (HTS) and High Content Assays (HCA) technologies are seen as key elements for filling the drug discovery pipeline in industry with new therapeutic compounds and new modes of action. However, neurons grown in vitro on standard HTS and HCA multi-well plates self-organize in each well in random entangled networks, the neuronal organization in each well is unique and difficult to reproduce, besides, this random organization lacks the characteristic compartmentalization of axons and soma seen in vivo. As a result, it is not possible to separate and easily identify axons for compatibility with HTS and HCA analysis of axons in culture. In addition, the large volume of medium on top of cells in standard multi-well neuronal cultures (ratio of cell volume to medium volume is over 1:200) hinders the homogeneous distribution of compounds secreted by the cells. Some cells grow in isolation while other cells grow too close to each other, resulting in random, non-organotypic structures unique to each well plate and difficult to standardize and reproduce. Therefore, neurons cultured in standard multi-well plates are not well suited to model neurological diseases, innervation and to investigate the neuronal relation with other cellular types in co-cultures compatible with HTS and HCA.

There is thus a need for technologies compatible with miniaturization, laboratory automation, and robotics to enable testing of thousands to hundreds of thousand compounds in vitro by means of high throughput screening (HTS) and high content analysis (HCA), in neurobiology as well as in other fields.

Recent innovations in microfluidic technology have been applied to compartmentalize and organize neuronal cell cultures [Magdesian MH, Anthonisen, M., Lopez-Ayon GM, Chua XY, Rigby M, Grütter P. (2017) Rewiring Neuronal Circuits: A New Method for Fast Neurite Extension and Functional Neuronal Connection. J Vis Exp. 2017 Jun 13;(124); Magdesian MH, Lopez-Ayon GM, Mori M, Boudreau D, Goulet-Hanssens A, Sanz R, Miyahara Y, Barrett CJ, Fournier AE, De Koninck Y, Grütter P. (2016) Rapid Mechanically Controlled Rewiring of Neuronal Circuits. J Neurosci. Jan 20;36(3):979-87; Anne M Taylor, Mathew Blurton-Jones, Seog Woo Rhee, David H Cribbs, Carl W Cotman, and N. L. J. A microfluidic culture platform for CNS axonal injury, regeneration, and transport. Nat. Methods 2, 599-605 (2005); In-mold patterning and actionable axo-somatic compartmentalization for on-chip neuron culture; Yamada A, Vignes M, Bureau C, Mamane A, Venzac B, Descroix S, Viovy JL, Villard C, Peyrin JM, Malaquin L. Lab Chip. 2016 May 24;16(11):2059-68.]

Also, various devices and chips using microfluidic technology have been developed and are described in Pats. US 10,526,572; US 9,637,715; US 10,532,355; US 7,419,822; EP 1 581 612; and in Pat. Publications US 2020/0208089; US 2020/0063081; WO 2020/074592. However, these systems have many deficiencies either because they are not efficient, they still require a high volume of cells and reagents, they require an active flow, they are difficult to implement, they are complex to use and require specialized training, they lack scalability and/or because they are not compatible with standard plates and equipment for high capacity assays and automation such as for HTS and HCA.

There is thus a need for multi-well devices and methods for analysis of cells, including neuronal cells, that are capable of supporting HTS and HCA analysis for research and drug discovery.

There is more particularly a need for microfluidic devices and methods that are easy to use and implement, do not require specialized training, and that are compatible with standard HTS and HCA automation equipment used at industrial scale and high capacity testing.

There is also a need for microplates that can be used for the culture and/or analysis of cells, including neurons, that are compliant with standard multi-well plates as defined by the Society for Laboratory Automation and Screening (ANSI/SLAS), and that enable faster, more reproducible, and standardized tests for multiple applications including, but not limited to drug screening, neurotoxicity tests, disease modelling, neurodevelopmental studies, axonal transport, etc.

### SUMMARY

In accordance with an aspect, there is provided A multi-well device for analysis of cells, comprising:
a multi-well grid layer comprising a plurality of wells;
a microfluidic layer comprising microchannels, wherein the microfluidic layer is configured for being positioned beneath the upper multi-well grid layer; and
a base layer configured for being positioned beneath the microfluidic layer and adapted for being detachably connected to the microfluidic layer and/or to the multi-well grid layer;
wherein once connected, the multi-well grid layer, the microfluidic layer and the base layer form at least one microchannel network enabling a fluid to flow therein from via the microchannels.

In some implementations, the microfluidic layer comprises a microfluidic unit comprising a central main chamber with microchannels, at least one inlet, at least one outlet and arms extending from the main chamber to the at least one inlet and the at least one outlet, the arms providing a fluidic communication between the central main chamber, the at least one inlet and the at least one outlet.

In some implementations, the at least one inlet, the at least one outlet, the central main chamber, the arms, and the microchannels are carved, printed, embossed, or moulded into the microfluidic layer.

In some implementations, the microfluidic layer comprises an upper surface that is bounded to a lower surface of the multi-well grid layer.

In some implementations, the multi-well grid layer comprises at least 6, 12, 24, 48, 96, 384, 1536 or 3456 wells.

In some implementations, at least one of the multi-well grid layers, the microfluidic layer and the base layer is made of glass and/or a polymeric material.

In some implementations, the base layer is made of an optically transparent material or a translucent material.

In some implementations, the optically transparent material is selected from the group consisting of glass, acrylic, polystyrene (PS), cyclo-olefin-copolymer (COC), cycloolefin polymer (COP), a thermoplastic elastomer (TPE) and polydimethylsiloxane (PDMS).

In some implementations, the base layer is coated with a substance that promotes cellular adhesion, promotes cellular growth or repels cellular adhesion.

In some implementations, the base layer comprises connecting means for detachably connecting the base layer to the multi-well grid layer.

In some implementations, the base layer comprises a frame and a transparent layer bonded to the frame.

In some implementations, the base layer comprises a frame and a transparent layer integral to the frame.

In some implementations, the multi-well device further comprises a lid adapted to be deposited over the multi-well grid layer.

In some implementations, the multi-well device is adapted for optical analysis of cells loaded into the at least one microchannel network.

In some implementations, the microfluidic layer is integral with the base layer.

In some implementations, the microfluidic layer is integral with the multi-well grid layer.

In some implementations, the microfluidic layer comprises a plurality of layers configured such that once superposed, the multi-well grid layer, the microfluidic layer, and the base layer form the least one microchannel network.

In some implementations, at least one layer of the plurality of layers is integral with the base layer.

In some implementations, at least one layer of the plurality of layers is integral with the multi-well grid layer.

In accordance with another aspect, there is provided a multi-well device for analysis of cells, comprising:
a multi-well grid layer comprising a plurality of wells;
a patterned layer configured for being positioned beneath the upper multi-well grid layer; and
a base layer configured for being positioned beneath the patterned layer and adapted for being detachably connected to the patterned layer and/or to the multi-well grid layer;
wherein once connected, the multi-well grid layer, the patterned layer and the base layer form at least one fluidic network enabling a fluid to flow therein.

In some implementations, the patterned layer comprises a patterned unit.

In some implementations, the patterned unit comprises a hole extending through a thickness of the patterned layer.

In some implementations, the hole is an inlet configured to receive a fluid therein.

In some implementations, the hole is an outlet configured for retrieving a fluid therefrom.

In some implementations, the patterned unit comprises a plurality of holes extending through a thickness of the patterned layer, the plurality of holes comprising an inlet to receive a fluid therein and an outlet configured for retrieving a fluid therefrom.

In some implementations, the inlet and the outlet are in fluid communication with a central main chamber.

In some implementations, the inlet and the outlet are in fluid communication with the central main chamber via a corresponding arm.

In some implementations, the patterned unit comprises an additional inlet and an additional outlet in fluid communication with the central main chamber.

In some implementations, the patterned unit is carved, printed, embossed, or moulded into the patterned layer.

In some implementations, the patterned unit comprises a microfluidic unit.

In some implementations, the microfluidic unit comprises a central main chamber with microchannels, and an inlet and an outlet both in fluid communication with the central main chamber.

In some implementations, the inlet and the outlet are in fluid communication with the central main chamber via a corresponding arm.

In some implementations, the microfluidic unit is carved, printed, embossed, or moulded into the microfluidic layer.

In some implementations, the patterned layer comprises an upper surface that is bounded to a lower surface of the multi-well grid layer.

In some implementations, the multi-well grid layer comprises at least 6, 12, 24, 48, 96, 384, 1536 or 3456 wells.

In some implementations, at least one of the multi-well grid layers, the patterned layer and the base layer is made of glass and/or a polymeric material.

In some implementations, the base layer is made of an optically transparent material or a translucent material.

In some implementations, the optically transparent material is selected from the group consisting of glass, acrylic, polystyrene (PS), cyclo-olefin-copolymer (COC), cycloolefin polymer (COP), a thermoplastic elastomer (TPE) and polydimethylsiloxane (PDMS).

In some implementations, the base layer is coated with a substance that promotes cellular adhesion, promotes cellular growth or repels cellular adhesion.

In some implementations, the base layer comprises connecting means for detachably connecting the base layer to the multi-well grid layer.

In some implementations, the base layer comprises a frame and a transparent layer bonded to the frame.

In some implementations, the base layer comprises a frame and a transparent layer integral to the frame.

In some implementations, the multi-well device further comprises a lid adapted to be deposited over the multi-well grid layer.

In some implementations, the multi-well device is adapted for optical analysis of cells loaded into the at least one microchannel network.

In some implementations, the patterned layer is integral with the base layer.

In some implementations, the patterned layer is integral with the multi-well grid layer.

In some implementations, the patterned layer comprises a plurality of layers configured such that once superposed, the multi-well grid layer, the patterned layer, and the base layer form the least one microchannel network.

In some implementations, at least one layer of the plurality of layers is integral with the base layer.

In some implementations, at least one layer of the plurality of layers is integral with the multi-well grid layer.

In accordance with another aspect, there is provided a method for culturing cells, comprising:
providing a microfluidic assembly comprising:
   a multi-well grid layer comprising a plurality of wells; and
   a microfluidic layer comprising microchannels, the microfluidic layer being positionable beneath the upper multi-well grid layer;
providing a base layer positionable beneath the microfluidic layer and adapted for being detachably connected to the microfluidic layer and/or to the multi-well grid layer;
connecting the base layer to the microfluidic assembly, wherein once connected the multi-well grid layer, the microfluidic layer, the base layer form at least one microchannel network enabling a fluid to flow therein via the microchannels; and
loading cells to be cultured into at least one well of the plurality of wells.

In some implementations, the method further comprises analyzing the cells loaded into the at least one well.

In some implementations, the cells are cultured for a certain period of time prior to the analysis.

In some implementations, analyzing the cells loaded into the at least one well comprises performing at least one of microscopy, electrical stimulation, absorbance, spectrophotometry, mass spectroscopy, or electrical impedance.

In some implementations, the at least one microchannel network comprises a central main chamber in fluid communication with at least one inlet and at least one outlet, and wherein the analysis of the cells loaded into the at least one well is carried out by analyzing cells that are in the central main chamber.

In some implementations, the multi-well grid layer comprises at least 6, 12, 24, 48, 96, 384, 1536 or 3456 wells.

In some implementations, the microfluidic assembly is adapted for high throughput optical analysis.

In some implementations, the method further comprises, prior to loading the cells, coating the base layer with a substance that promotes cellular adhesion, promotes cellular growth or repels cellular adhesion.

In some implementations, the method further comprises detaching the microfluidic assembly from the base layer, leaving organized cells attached to the base layer.

In some implementations, the method is for drug discovery, drug screening and/or systems biology.

In accordance with another aspect, there is provided a kit for analysis of cells, comprising:
a fluidic assembly comprising:
   a multi-well grid layer comprising a plurality of wells; and
   a patterned layer comprising microchannels and configured to be positioned beneath the upper multi-well grid layer; and
a base layer configured for being positioned beneath the patterned layer and adapted for being detachably connected to the patterned layer and/or to the multi-well grid layer; wherein once connected, the multi-well grid layer, the microfluidic layer, and the base layer
form at least one microchannel network enabling a fluid to flow therein via the microchannels.

In some implementations, the kit further comprises at least one feature as described herein.

In accordance with another aspect, there is provided a multi-well device for analysis of cells comprising:
a microfluidic layer comprising:
   a central main chamber comprising a first compartment and a second compartment, wherein the first and second compartments are separated by a plurality of microfluidic channels, the microfluidic channels providing a fluidic communication between the first and second compartments;
   a first inlet and a first outlet disposed at opposite ends of the first compartment of the central main chamber;
   two arms extending diagonally in opposite directions from the first compartment of the central main chamber, the arms providing a fluidic communication of the first inlet and the first outlet with the first compartment of the central main chamber;
   a second inlet and a second outlet disposed at opposite ends of the second compartment of the central main chamber;
   two arms extending diagonally in opposite directions from the second compartment of the central main chamber, the arms providing a fluidic communication of the second inlet and the second outlet with the second compartment of the central main chamber;
a multi-well grid layer configured to be superposed over the microfluidic layer;
the multi-well grid layer comprising a corresponding well extending therethrough and vertically aligned with the central main chamber, the first inlet, the first outlet, the second inlet and the second outlet of the microfluidic layer, respectively;
a base layer configured for being positioned beneath the microfluidic layer.

In some implementations, the multi-well grid layer comprises at least nine (9) wells that are distributed in a 3 × 3 configuration, and wherein said 3 × 3 configuration comprises (i) a center well vertically aligned over the main chamber and (ii) four opposite corner wells vertically aligned over the first inlet, the first outlet, the second inlet and the second outlet of the microfluidic layer, respectively.

In some implementations, the central main chamber, the first inlet, the first outlet, the second inlet and the second outlet of the microfluidic layer forms together a single microfluidic unit having an X-configuration.

In some implementations, the base layer is detachably connected to the microfluidic layer and/or to the multi-well grid layer.

In some implementations, the microfluidic layer comprises an upper surface that is bounded to a lower surface of the multi-well grid layer.

In some implementations, the multi-well grid layer comprises at least 6, 12, 24, 48, 96, 384, 1536 or 3456 wells.

In some implementations, at least one of the multi-well grid layer, the microfluidic layer and the base layer is made of glass and/or a polymeric material.

In some implementations, the base layer is transparent or translucid.

In accordance with another aspect, there is provided a device for analysis of cells, comprising:
a microfluidic layer configured for being placed in contact with an electrode layer comprising electrodes, the microfluidic layer comprising a microfluidic unit having microchannels configured to receive at least a component of the cells therein, the microchannels being provided in a spaced-apart relationship relative to each other; and
an alignment feature for aligning the microchannels of the microfluidic unit with the electrodes of the electrode layer once the microfluidic layer is placed in contact with the electrode layer to achieve a predetermined organized architecture of the microchannels relative to the electrodes.

In some implementations, each of the electrodes comprises an electrode tip, and the alignment feature enables alignment of at least one microchannel with a predetermined number of the electrode tips.

In some implementations, each of the electrodes comprises an electrode tip, and the alignment feature enables alignment of a predetermined number of the electrode tips laterally along the microchannels.

In some implementations, each of the electrodes comprises an electrode tip, and the alignment feature enables positioning of a predetermined number of the microchannels over a predetermined number of electrode tips.

In some implementations, each of the electrodes comprises an electrode tip, and the alignment feature enables placement of the microfluidic layer over the electrode layer such that the microchannels extend substantially vertically or substantially horizontally and intersect a predetermined number of electrode tips.

In some implementations, each of the electrodes comprises an electrode tip, and the alignment feature enables positioning the microfluidic layer such that the microchannels intersect the electrode tips along an entire diameter of the electrode tips.

In some implementations, the microfluidic layer comprises a plurality of microfluidic units, each microfluidic unit of the plurality of microfluidic units being associated with a corresponding electrode grid of the electrode layer.

In some implementations, the alignment feature comprises a microfluidic layer alignment opening defined in the microfluidic layer, the microfluidic layer alignment opening being engageable with an electrode layer protruding member extending upwardly from the electrode layer.

In some implementations, the alignment feature comprises a microfluidic layer protruding member extending downwardly from the microfluidic layer, the microfluidic layer protruding member being engageable with an electrode layer alignment cavity defined in the electrode layer.

In some implementations, the alignment feature comprises a series of ridges protruding from a lower surface of the microfluidic layer, the series of ridges being engageable with a complimentary series of furrows defined in an upper surface of the electrode layer.

In some implementations, the alignment feature comprises an alignment marking provided on the microfluidic layer, the alignment marking having a predetermined configuration based on a distribution of the electrodes of the electrode grid to enable alignment of the microchannels with the electrodes.

In some implementations, the alignment feature comprises a microfluidic layer alignment frame coupled to the microfluidic layer, the microfluidic layer alignment frame being configured to engage with an electrode layer frame to secure the microfluidic layer in the predetermined organized architecture of the microchannels relative to the electrodes.

In some implementations, the microfluidic layer alignment frame is engageable with the electrode layer frame via a snap-on mechanism.

In some implementations, the microfluidic layer comprises a single microfluidic unit, the single microfluidic unit being associated with a corresponding electrode grid of the electrode layer.

In some implementations, the alignment feature comprises a microfluidic layer alignment opening defined in the microfluidic layer, the microfluidic layer alignment opening being engageable with an electrode layer protruding member extending upwardly from the electrode layer.

In some implementations, the alignment feature comprises a microfluidic layer protruding member extending downwardly from the microfluidic layer, the microfluidic layer protruding member being engageable with an electrode layer alignment cavity defined in the electrode layer.

In some implementations, the alignment feature comprises a series of ridges protruding from a lower surface of the microfluidic layer, the series of ridges being engageable with a complimentary series of furrows defined in an upper surface of the electrode layer.

In some implementations, the alignment feature comprises an alignment marking provided on the microfluidic layer, the alignment marking having a predetermined configuration based on a distribution of the electrodes of the electrode grid to enable alignment of the microchannels with the electrodes.

In some implementations, the alignment feature comprises a microfluidic layer alignment frame coupled to the microfluidic layer.

In some implementations, the microfluidic layer alignment frame is configured to engage with a peripheral wall of a well having the electrode layer as a bottom wall.

In some implementations, the microfluidic layer alignment frame comprises an alignment tab configured to be received within an alignment tab-receiving cavity defined in the peripheral wall of the well.

In some implementations, the microfluidic layer alignment frame comprises a predetermined number of alignment tabs configured to be received in a corresponding predetermined number of alignment tab-receiving cavities defined in the peripheral wall of the well.

In some implementations, the microfluidic layer alignment frame extends upwardly from the microfluidic layer, and the alignment tabs are provided in an upper portion of the microfluidic layer alignment frame.

In some implementations, the microfluidic layer alignment frame at least partially surrounds an outer periphery of the microfluidic layer.

In some implementations, the microfluidic layer alignment frame is engageable with an electrode layer frame via a snap-on mechanism.

In some implementations, the at least a component of the cells received in the microchannels comprises axons of neuronal cells.

In accordance with another aspect, there is provided a device for establishing electrical communication with cells, comprising:
an electrode layer configured for being placed in contact with a microfluidic layer comprising a microfluidic unit having microchannels configured to receive at least a component of the cells therein, the electrode layer comprising electrodes for interacting with at least a component of cells received in the microchannels; and
an alignment feature for aligning the electrodes with the microchannels of the microfluidic unit once the electrode layer is placed in contact with the microfluidic layer to achieve a predetermined organized architecture of the microchannels relative to the electrodes.

In some implementations, each of the electrodes comprises an electrode tip, and the alignment feature enables alignment of a predetermined number of the electrode tips with at least one microchannel.

In some implementations, each of the electrodes comprises an electrode tip, and the alignment feature enables alignment of a predetermined number of the electrode tips laterally along the microchannels.

In some implementations, each of the electrodes comprises an electrode tip, and the alignment feature enables positioning of a predetermined number of electrodes tips over a predetermined number of microchannels.

In some implementations, each of the electrodes comprises an electrode tip, and the alignment feature enables placement of the electrode layer in contact with the microfluidic layer such that a predetermined number of the electrode tips intersect the microchannels.

In some implementations, each of the electrodes comprises an electrode tip, and the alignment feature enables positioning the electrode layer such that the microchannels intersect the electrode tips along an entire diameter of the electrode tips.

In some implementations, the electrodes of the electrode layer are provided as a plurality of electrode grids that are placeable in contact with a corresponding microfluidic unit of the microfluidic layer.

In some implementations, the alignment feature comprises an electrode layer protruding member extending upwardly from the electrode layer, the electrode layer protruding member being engageable with a microfluidic layer alignment opening defined in the microfluidic layer.

In some implementations, the alignment feature comprises an electrode layer alignment cavity defined in the electrode layer, the electrode layer alignment cavity being engageable with a microfluidic layer protruding member extending downwardly from the microfluidic layer.

In some implementations, the alignment feature comprises a series of ridges protruding from an upper surface of the electrode layer, the series of ridges being engageable with a complimentary series of furrows defined in a lower surface of the microfluidic layer.

In some implementations, the alignment feature comprises an electrode layer alignment frame surrounding the electrode layer, the electrode layer alignment frame being configured to engage with a microfluidic layer alignment frame to secure the electrode layer in the predetermined organized architecture of the microchannels relative to the electrodes.

In some implementations, the electrode layer alignment frame is engageable with the microfluidic layer alignment frame via a snap-on mechanism.

In some implementations, the electrode layer is provided as a bottom wall of a well of a multi-well plate.

In some implementations, the electrodes of the electrode layer are provided as an electrode grid.

In some implementations, the alignment feature comprises an electrode layer protruding member extending upwardly from the electrode layer, the electrode layer protruding member being engageable with a microfluidic layer alignment opening defined in the microfluidic layer.

In some implementations, the alignment feature comprises an electrode layer alignment cavity defined in the electrode layer, the electrode layer alignment cavity being engageable with a microfluidic layer protruding member extending downwardly from the microfluidic layer.

In some implementations, the alignment feature comprises a series of ridges protruding from an upper surface of the electrode layer, the series of ridges being engageable with a complimentary series of furrows defined in a lower surface of the microfluidic layer.

In some implementations, the alignment feature comprises an alignment tab-receiving cavity defined in a peripheral wall of the well, the alignment tab-receiving cavity being configured to receive therein an alignment tab extending from a microfluidic layer alignment frame coupled to the microfluidic layer.

In some implementations, the peripheral wall of the well comprises a predetermined number of alignment tab-receiving cavities for receiving a corresponding predetermined number of alignment tabs tab extending from a microfluidic layer alignment frame coupled to the microfluidic layer.

In some implementations, the alignment tab-receiving cavity is provided in an upper portion of the well.

In some implementations, the at least a component of the cells received in the microchannels comprises axons of neuronal cells.

In accordance with another aspect, there is provided a device for analysis of cells, comprising:
an electrode layer comprising electrodes for establishing electrical communication with the cells;
a microfluidic layer configured for being placed in contact with the electrode layer, the microfluidic layer comprising a microfluidic unit having microchannels configured to receive at least a component of the cells therein, the microchannels being provided in a spaced-apart relationship relative to each other; and
an alignment feature for aligning the microchannels of the microfluidic unit with the electrodes of the electrode layer once the microfluidic layer is placed in contact with the electrode layer to achieve a predetermined organized architecture of the microchannels relative to the electrodes.

In some implementations, the device further comprises one or more features as defined herein.

In accordance with another aspect, there is provided a method for placing a microfluidic layer in contact with an electrode layer, the method comprising:
placing the microfluidic layer in proximity of the electrode layer; and
aligning microchannels of a microfluidic unit of the microfluidic layer with electrodes of the electrode layer using an alignment feature to achieve a predetermined organized architecture of the microchannels relative to the electrodes, the microchannels being configured to receive at least a component of cells therein and being provided in a spaced-apart relationship relative to each other.

In some implementations, each of the electrodes comprises an electrode tip, and aligning the microchannels of the microfluidic unit with the electrodes of the electrode layer using the alignment feature comprises aligning at least one microchannel with a predetermined number of the electrode tips.

In some implementations, each of the electrodes comprises an electrode tip, and aligning the microchannels of the microfluidic unit with the electrodes of the electrode layer using the alignment feature comprises aligning a predetermined number of the electrode tips laterally along the microchannels.

In some implementations, each of the electrodes comprises an electrode tip, and aligning the microchannels of the microfluidic unit with the electrodes of the electrode layer using the alignment feature comprises positioning a predetermined number of the microchannels over a predetermined number of electrode tips.

In some implementations, each of the electrodes comprises an electrode tip, and aligning the microchannels of the microfluidic unit with the electrodes of the electrode layer using the alignment feature comprises placing the microfluidic layer over the electrode layer such that the microchannels extend substantially vertically or substantially horizontally and intersect a predetermined number of electrode tips.

In some implementations, each of the electrodes comprises an electrode tip, and aligning the microchannels of the microfluidic unit with the electrodes of the electrode layer using the alignment feature comprises positioning the microfluidic layer such that the microchannels intersect the electrode tips along an entire diameter of the electrode tips.

In some implementations, aligning the microchannels of the microfluidic unit with the electrodes of the electrode layer using the alignment feature comprises engaging a microfluidic layer alignment opening defined in the microfluidic layer with an electrode layer protruding member extending upwardly from the electrode layer.

In some implementations, aligning the microchannels of the microfluidic unit with the electrodes of the electrode layer using the alignment feature comprises engaging a microfluidic layer protruding member extending downwardly from the microfluidic layer with an electrode layer alignment cavity defined in the electrode layer.

In some implementations, aligning the microchannels of the microfluidic unit with the electrodes of the electrode layer using the alignment feature comprises engaging a series of ridges protruding from a lower surface of the microfluidic layer with a complimentary series of furrows defined in an upper surface of the electrode layer.

In some implementations, aligning the microchannels of the microfluidic unit with the electrodes of the electrode layer using the alignment feature comprises aligning an alignment marking provided on the microfluidic layer with the electrodes, the alignment marking having a predetermined configuration based on a distribution of the electrodes.

In some implementations, aligning the microchannels of the microfluidic unit with the electrodes of the electrode layer using the alignment feature comprises engaging an alignment tab of a microfluidic layer alignment frame coupled to the microfluidic layer with an alignment tab-receiving cavity defined in a peripheral wall of a well having the electrode layer as a bottom wall.

In some implementations, aligning the microchannels of the microfluidic unit with the electrodes of the electrode layer using the alignment feature comprises engaging a microfluidic layer alignment frame coupled to the microfluidic layer with an electrode layer frame surrounding the electrode layer.

In some implementations, the microfluidic alignment frame is engageable with the electrode layer frame via a snap-on mechanism.

In some implementations, the at least a component of the cells received in the microchannels comprises axons of neuronal cells.

In accordance with another aspect, there is provided a multi-well device for analysis of cells, comprising:
a multi-well grid layer comprising a plurality of wells;
a microfluidic layer comprising a microfluidic unit having microchannels configured to receive at least a component of the cells therein, the microchannels being provided in a spaced-apart relationship relative to each other; and
an electrode layer comprising electrodes placeable in contact with the microfluidic layer to achieve a predetermined organized architecture of the microchannels relative to the electrodes.

In some implementations, the multi-well device further comprises a base layer positionable underneath the electrode layer, the base layer being detachably connectable to at least one of the microfluidic layer, the electrode layer or the multi-well grid layer.

In some implementations, the multi-well device further comprises a base layer positionable underneath the microfluidic layer, the electrode layer being integrated into the base layer.

In some implementations, the multi-well device further comprises one or more features as defined herein.

In accordance with another aspect, there is provided a device for analysis of cells, comprising:
a plurality of microfluidic layers each comprising a microfluidic unit having microchannels configured to receive at least a component of the cells therein; and
a microfluidic layer engaging frame engageable with the plurality of microfluidic layers such that once engaged, the microfluidic layer engaging frame and the plurality of microfluidic layers form a unitary structure, the unitary structure being engageable with a multi-well plate comprising a plurality of wells each comprising an electrode layer having electrodes and each being configured for receiving therein a corresponding one of the plurality of microfluidic layers to place the corresponding one of the plurality of microfluidic layers in contact with the electrode layer to achieve a predetermined organized architecture of the microchannels relative to the electrodes.

In some implementations, the microfluidic layer engaging frame comprises a base wall comprising microfluidic layer openings defined therethrough to enable fluid communication with the microfluidic unit and insertion and/or removal of fluids into the microfluidic unit.

In some implementations, the microfluidic layer engaging frame comprises a multi-well plate alignment feature extending downwardly toward the multi-well plate, the multi-well plate alignment feature being insertable into an alignment feature receiving opening defined in the multi-well plate to align the microfluidic layer engaging frame with the multi-well plate.

In some implementations, the microfluidic layer engaging frame comprises an engagement feature engageable with an engagement feature connector of the multi-well plate.

In some implementations, the engagement feature is engageable with the engagement feature connector of the multi-well plate via a snap-on mechanism.

In some implementations, wherein the plurality of microfluidic layers is integral with the microfluidic layer engaging frame.

In some implementations, the microfluidic layer engaging frame comprises a microfluidic layer alignment feature configured for placement of the corresponding one of the plurality of microfluidic layers at a given location of the microfluidic layer engaging frame.

In accordance with another aspect, there is provided a device for analysis of cells, comprising:
a microfluidic layer comprising a plurality of microfluidic units each comprising microchannels; and
a multi-well grid layer comprising a plurality of bottomless wells, the multi-well grid layer being positionable over the microfluidic layer; and
a well identification feature provided on an upper surface of the multi-grid layer, the well identification feature being associated with a corresponding microfluidic unit of the plurality of microfluidic units to enable visual identification of at least one predetermined well of the multi-well grid layer that is in fluid communication with a component of the corresponding microfluidic unit.

In some implementations, each microfluidic unit comprises:
first and second inlets;
first and second outlets, the first outlet being in fluid communication with the first inlet via a first compartment and the second outlet being fluid communication with the second inlet via a second compartment;
wherein the microchannels extend between the first and second compartments.

In some implementations, the well identification feature comprises a well marking.

In some implementations, the well marking comprises an individual well marking associated with each one of the first and second inlets and the first and second outlets once the multi-well grid layer is positioned over the microfluidic layer, each one of the first and second inlets and the first and second outlets corresponding to a respective component of the microfluidic layer.

In some implementations, the at least one predetermined well of the multi-well grid layer comprises a plurality of wells associated with the corresponding microfluidic unit, and the well marking comprises an outer well marking provided at an outer periphery of the plurality of wells of the multi-well grid layer to visually identify the corresponding microfluidic unit once the multi-well grid layer is positioned over the microfluidic layer.

In some implementations, the well identification feature comprises a well identification layer superposable to an upper surface of the multi-well grid layer.

In some implementations, the well identification layer comprises columns provided in between longitudinally spaced-apart microfluidic units of the plurality of microfluidic units.

In some implementations, the well identification layer comprises rows provided in between laterally spaced-apart microfluidic units of the plurality of microfluidic units.

In accordance with another aspect, there is provided a method for culturing cells, comprising:
providing a fluidic assembly comprising:
   a multi-well grid layer comprising a plurality of wells; and
   a patterned layer being positionable beneath the upper multi-well grid layer;
providing a base layer positionable beneath the patterned layer and adapted for being detachably connected to the patterned layer and/or to the multi-well grid layer;
connecting the base layer to the fluidic assembly, wherein once connected the multi-well grid layer, the patterned layer, the base layer form at least one fluidic network enabling a fluid to flow therein; and
loading cells to be cultured into at least one well of the plurality of wells.

### BRIEF DESCRIPTION OF THE DRAWINGS

The attached figures illustrate various features, aspects and implementations of the technology described herein.

FIG. 1 is a top exploded perspective view showing an implementation of a multi-well device for analysis of cells, the device comprising a multi-well grid layer, a microfluidic layer and a base layer.

FIGS. 2A-2C are, respectively, a top perspective view (FIG. 2A), a top view (FIG. 2B) and a side view (FIG. 2C) of the microfluidic layer of FIG. 1.

FIG. 3A is an enlarged top perspective view of a microfluidic unit of the microfluidic layer of FIG. 1, and a further enlarged perspective view of the microchannels of the microfluidic unit.

FIGS. 3B to 3F are top views and cross-sectional views of the microfluidic unit of FIG. 3A, taken along the dotted lines of each microfluidic pattern.

FIGS. 4A-4C are, respectively, a top perspective view (FIG. 4A), a top view (FIG. 4B), and a side view (FIG. 4C) of the base layer of FIG. 1.

FIG. 5 is an enlarged perspective view of the microfluidic unit of FIG. 3A, showing a flow in different compartments (white arrows).

FIG. 6A is a top perspective view of an optional transparent lid and FIG. 6B is top perspective view of the lid deposited over the multi-well device of FIG. 1.

FIG. 6C is a top view of the multi-well device of FIG. 1.

FIG. 6D is a bottom view of the multi-well device of FIG. 1.

FIGS. 7A-7D illustrate an implementation of a multi-well device using a 384-well plate, with FIG. 7A showing a multi-well grid layer comprising 24 microfluidic units, FIG. 7B showing an enlarged view of one of the 24 microfluidic units, FIG. 7C showing an enlarged view of an assay window composing the microfluidic unit, and FIG. 7D showing a picture of the assay window showing neurons in culture.

FIGS. 8A-8F is a panel of drawings illustrating examples of different configurations of microfluidic units disposed under a grid of a 384-well plate.

FIG. 9 is a panel providing examples of ranges of values for length and dimensions of microfluidic units adapted for compatibility with 24-well plates, 96-well plates and 384-well plates.

FIGS. 10A-10B show pictures of cultured neurons having longer parallel axons (FIG. 10A) or shorter parallel axons (FIG. 10B), in accordance with Example 1.

FIG. 11 show pictures of cultured neurons, in accordance with Example 2.

FIG. 12 is a picture of a co-culture of neurons (*) and astrocytes (**), in accordance with Example 3.

FIG. 13 is a portion of a microfluidic layer and an electrode layer, the electrode layer being superposed to the microfluidic layer and including a group of electrodes, each electrode including a tip.

FIG. 14 is a portion of a microfluidic layer and an electrode layer, the electrode layer being superposed to the microfluidic layer and including a group of electrodes, each electrode including a tip.

FIG. 15 is a prior art picture showing cultured neuronal cells randomly distributed with respect to the electrodes of an electrode layer.

FIG. 16 is a picture of axons growing according to an organized architecture in microchannels, and electrodes being aligned along the axons.

FIG. 17 is an exploded perspective view of a multi-well device as described herein, with an electrode layer shown underneath a microfluidic layer.

FIG. 18 is a top view of a microfluidic layer that includes 96 microfluidic units.

FIG. 19 is a top view of an electrode layer that include 96 electrode grids.

FIG. 20 is a top view of a microfluidic layer that includes 24 microfluidic units, the microfluidic layer being layered with an electrode layer that includes a corresponding 24 electrode grids, with an enlarged portion showing a microfluidic unit that includes microchannels and electrodes aligned along the microchannels.

FIG. 21 is a top view of a microfluidic unit that includes microchannels and electrodes aligned along the microchannels.

FIG. 22 is a top view of a microfluidic unit that includes microchannels and electrodes aligned along the microchannels.

FIG. 23 is a top view of a portion of a microfluidic layer that includes an alignment marking that includes a plurality of elongated markings.

FIG. 24 is a top view of a portion of a microfluidic layer that includes an alignment marking that includes a plurality of elongated markings, and electrodes aligned according to the alignment marking.

FIG. 25 is a cross-sectional view of an electrode grid of an electrode layer that includes a protective coating deposited onto the electrodes, and a base layer underneath the electrode layer.

FIG. 26 is a cross-sectional view of the electrode layer shown in FIG. 25, with a microfluidic layer being superposed onto the electrode layer, the microchannels being aligned with the electrodes.

FIG. 27 is a top view of an example of a multi-well plate that includes six wells, each of the wells including an electrode layer that is integrated into the bottom wall of the well.

FIG. 28 is a top view of a well of the multi-plate of FIG. 27 into which is received a microfluidic layer, and an enlarged view of the electrodes being aligned with the microchannels.

FIG. 28 is a top view of a well of the multi-plate of FIG. 27 into which is received a microfluidic layer, and an enlarged view of the electrodes being aligned with the microchannels.

FIG. 30 is, on a left-hand side, a top view of a schematic representation of the multi-plate of FIG. 27, showing one microfluidic layer received into one of the wells, the microfluidic layer being coupled to an alignment frame, and on the right-hand side, a top view and a side view of the alignment frame coupled to the microfluidic layer, a side view of a peripheral wall of a well of the multi-well plate, and a side view of the alignment frame and the microfluidic layer received into the well of the multi-well plate.

FIG. 31 is a perspective view of a multi-well grid layer.

FIG. 32 is a perspective view of the multi-well grid layer shown in FIG. 31, the multi-well grid layer including individual well markings and well markings.

FIG. 33 is a perspective view of the multi-well grid layer shown in FIG. 31, the multi-well grid layer including a well identification layer.

FIG. 34 is an exploded perspective view of a cell culture device that includes a microfluidic layer engagement frame that is engageable with a plurality of microfluidic layers each having a microfluidic unit.

FIG. 35 is a perspective view of the cell culture device of FIG. 34 showing the microfluidic layer engagement frame engaged with a plurality of microfluidic layers.

FIG. 36 is an exploded perspective view of the cell culture device of FIG. 35 and a multi-well plate that includes six wells, each of the wells including an electrode layer that is integrated into the bottom wall of the well.

FIG. 37 is an exploded perspective view of the cell culture device of FIG. 36.

FIG. 38 is a perspective view of the cell culture device of FIG. 37, shown with the microfluidic layer engagement frame engaged with the multi-well plate.

FIG. 39 is a top view of the microfluidic layer engagement frame and plurality of microfluidic layers of FIG. 35, with an enlarged section showing a microfluidic unit.

FIG. 40 is a top view of the multi-well plate of FIG. 36, with an enlarged section of electrodes of an electrode grid that are electrically connected by wires to a series of terminals.

FIG. 41 is a top view of the microfluidic layer engagement frame and plurality of microfluidic layers of FIG. 39 and the multi-well plate of FIG. 40 shown engaged and with the electrodes being aligned along the microchannels.

FIG. 42 is a top view of a section of the microfluidic layer engagement frame and plurality of microfluidic layers of FIG. 39 and the multi-well plate of FIG. 40 shown engaged and with the electrodes being aligned along the microchannels.

FIG. 43 is a perspective cross-sectional view of the microfluidic layer engagement frame and plurality of microfluidic layers of FIG. 39 and the multi-well plate of FIG. 40 shown engaged.

FIG. 44 is an exploded perspective view of a multi-well grid layer, a microfluidic layer that includes a first, second and third layer, and a base layer.

FIG. 45 is an exploded perspective view of a multi-well grid layer, a microfluidic layer that includes a first, second and third layer, an electrode layer, and a base layer.

### DETAILED DESCRIPTION

In the following description of the implementations, references to the accompanying drawings are illustrations of an example by which the technology may be practiced. It will be understood that other implementations may be made without departing from the scope of the technology disclosed.

### General Overview

The techniques described herein provide multi-well devices and methods for analysis of cells. The current technology is compatible with high capacity experimentation and automation equipment, significantly increasing efficiency of HTS and HCA.

In one particular aspect, the present technology involves microplates compliant with standard multi-well plates as defined by the Society for Laboratory Automation and Screening (ANSI/SLAS), wherein groups of wells are fluidically connected to guide a flow of liquid, which can include cells, for instance to a visualization window with precise microarchitecture allowing organization of cells (e.g., neuronal cells) for cultures and more efficient biological assays.

Although the present technology has first been developed with the focus of culture and analysis of neuronal cells, it is applicable to other fields of diagnostic, research and drug discovery including, but not limited to, infectious diseases, fertility (e.g., sperm analysis), cancer, muscular diseases, immune diseases, Alzheimer’s disease (e.g., neuronal cells), etc.

The current technology enables faster, more reproducible, and standardized tests and can be used for multiple applications including, but not limited to drug screening, neurotoxicity tests, disease modelling, neurodevelopmental studies, axonal transport, degeneration and regeneration, etc.

For instance, neurons cultured according to the present technology can be used for a wide range of cellular assays in neurobiology research and drug discovery. The precise architecture of neuronal cells that is obtained when cultured within the current technology allows simultaneous study of bundles of axons and single axons (in the corners of the seeding chamber, the neurons are denser and will form bundles, whereas in the middle single axons can be found in each channel).

### Multi-Well Device for Analysis of Cells

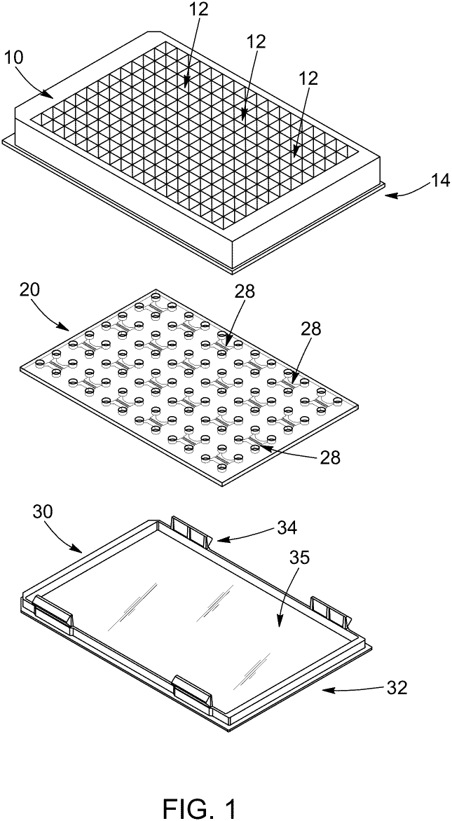

FIGS. 1 to 8 show implementations of a multi-well device for analysis of cells. In the illustrated implementations, the device 1 comprises: a multi-well grid layer 10 comprising a plurality of wells 12; a microfluidic layer 20 comprising a series of microfluidic units 28 with microchannels 65, and a base layer 30 configured for being positioned beneath the microfluidic layer 20 and adapted for being detachably connected, or assembled, to the microfluidic layer 20 and/or to the multi-well grid layer 10.

The multi-well grid layer 10 comprises a plurality of bottomless wells 12. In the illustrated implementations, the multi-well grid layer 10 comprises a hollow rectangular frame 14 surrounding a central portion comprising a plurality of wells 12 (e.g., a 384-well plate in the figures). Depending on the needs of a user, the multi-well grid layer 10 may comprise any number of wells, for instance 6, 12, 24, 48, 96, 384, or 1536 wells. In some implementations, the multi-well grid layer 10 can be a commercially available bottomless multi-well plate comprising 6, 12, 24, 48, 96, 384, or 1536 wells, and the microfluidic layer 20 is configured to comprise inlets, outlets and a central assay window that are vertically aligned with at least some of the bottomless wells 12 of the multi-grid layer 10 (see hereinafter for more information regarding configurations of the device). In some implementations, the multi-well grid layer 10 can be a commercially available bottomless multi-well plate which complies with American National Standards Institute of the Society for Laboratory Automation and Screening (ANSI/SLAS) microplate standards.

The microfluidic layer 20 can be integral to (e.g., moulded with) the multi-well grid layer 10. The microfluidic layer 20 can also be reversibly or irreversibly attached to the multi-well grid layer 10 by using any suitable method or technique, including but not limited to, compression, surface adhesion, ultrasonic welding, laser welding, thermocompression bonding, plasma bonding, solvent-assisted bonding, laser-assisted bonding or adhesive bonding using glue, pressure sensitive adhesives, or double-sided adhesive tape.

The base layer 30 is configured for being positioned beneath the microfluidic layer 20, and can be adapted for being detachably connected, or assembled, to the microfluidic layer 20 and/or to the multi-well grid layer 10. FIG. 1 illustrates an example in which the base layer 30 is positioned underneath the microfluidic layer 20, which is itself positioned underneath the multi-well grid layer 10.

Alternatively, in other implementations, the microfluidic layer 20 can be integral to (e.g., moulded with) the base layer 30. The microfluidic layer 20 can be reversibly or irreversibly attached to the base layer 30 by using any suitable method or technique, including but not limited to, compression, surface adhesion, ultrasonic welding, laser welding, thermocompression bonding, plasma bonding, solvent-assisted bonding, laser-assisted bonding or adhesive bonding using glue, pressure sensitive adhesives, or double-sided adhesive tape.

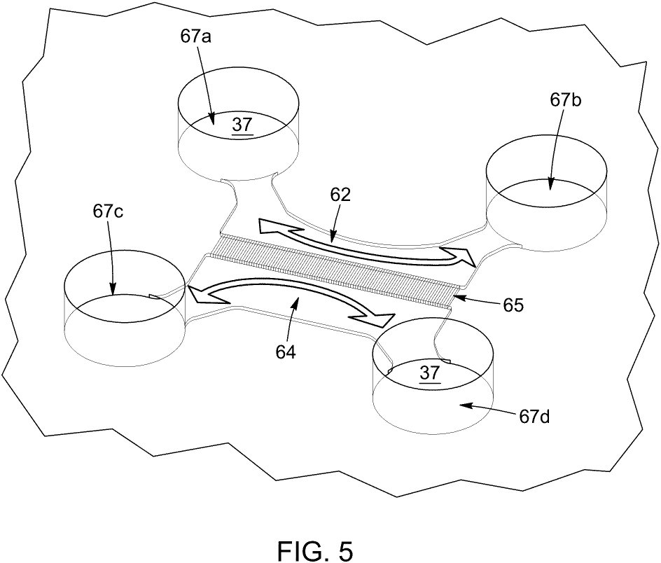

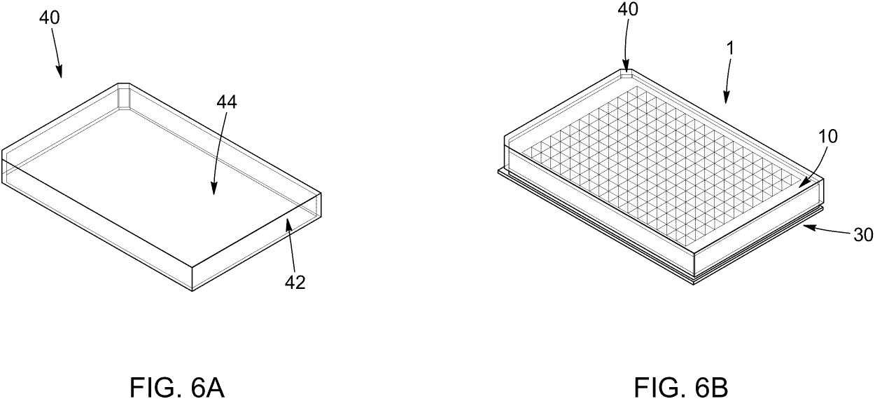

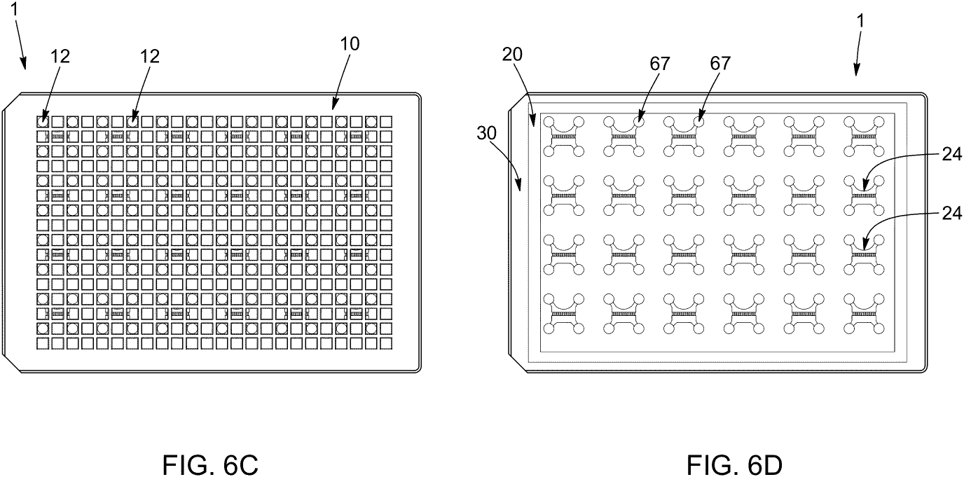

As shown FIG. 5 and FIGS. 6C-6D, once the multi-well grid 10, the microfluidic layer 20 and the base layer 30 are superposed to each other, such as when they are connected or assembled together, the multi-well grid layer 10, the microfluidic layer 20, and the base layer 30 form at least one microchannel network 24 (typically many networks) into which a fluid can flow, e.g., in a substantially leak-tight manner, from at least one well 12 of the multi-well grid layer 10 to at least another well 12 (e.g., via inlets/outlets 67a, 67b, 67c, 67d, the compartments 62, 64 and also the microchannels 65 composing the microfluidic layer 20). Therefore, liquids, particles and cells can advantageously flow in the microfluidic unit 28 using passive flow, without the need of pumps, with the microchannels 65 being positioned substantially transversally to the direction of the fluidic flow from the inlet to the outlet.

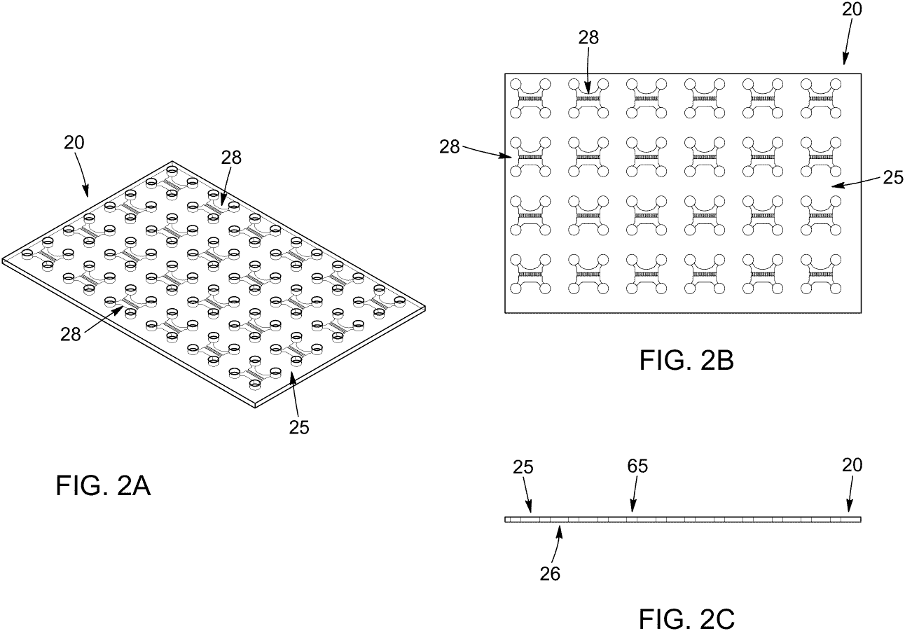

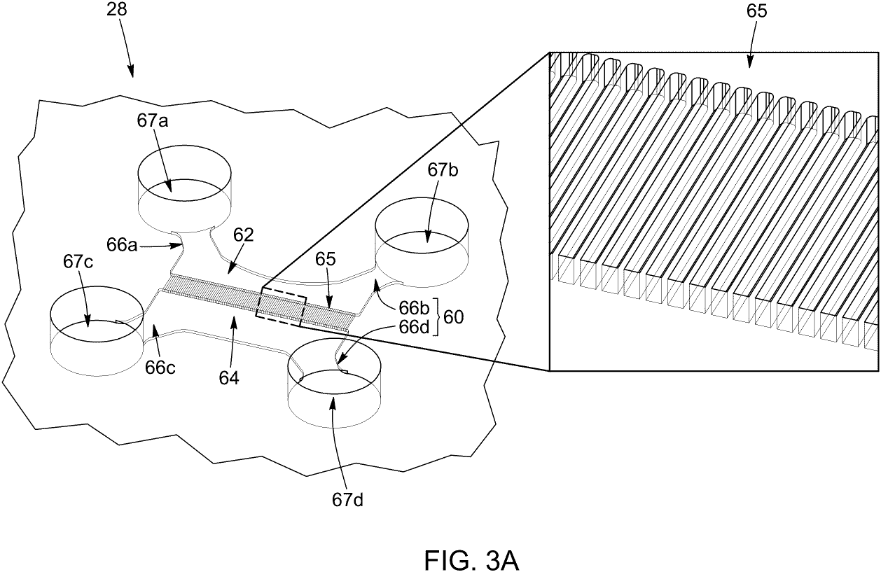

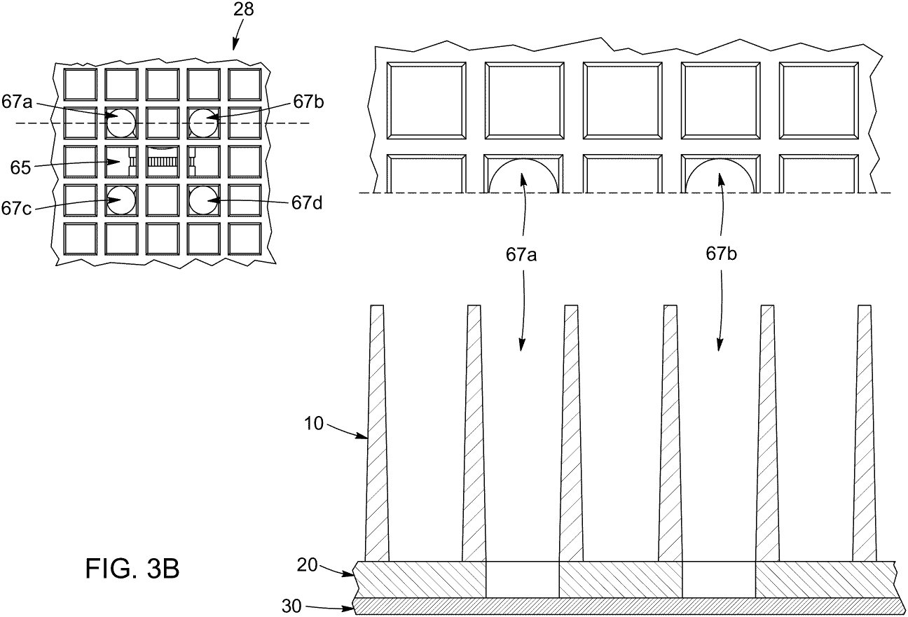

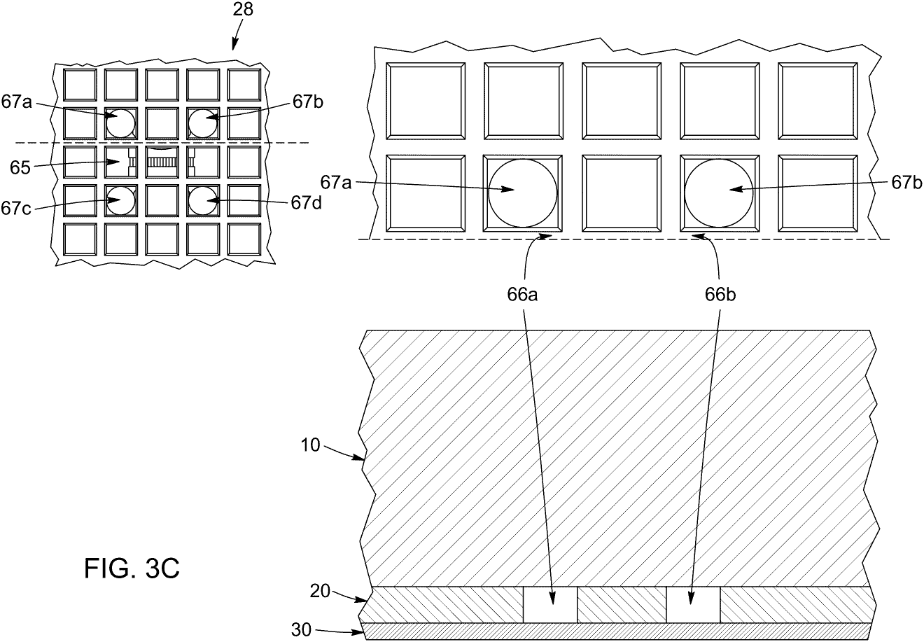

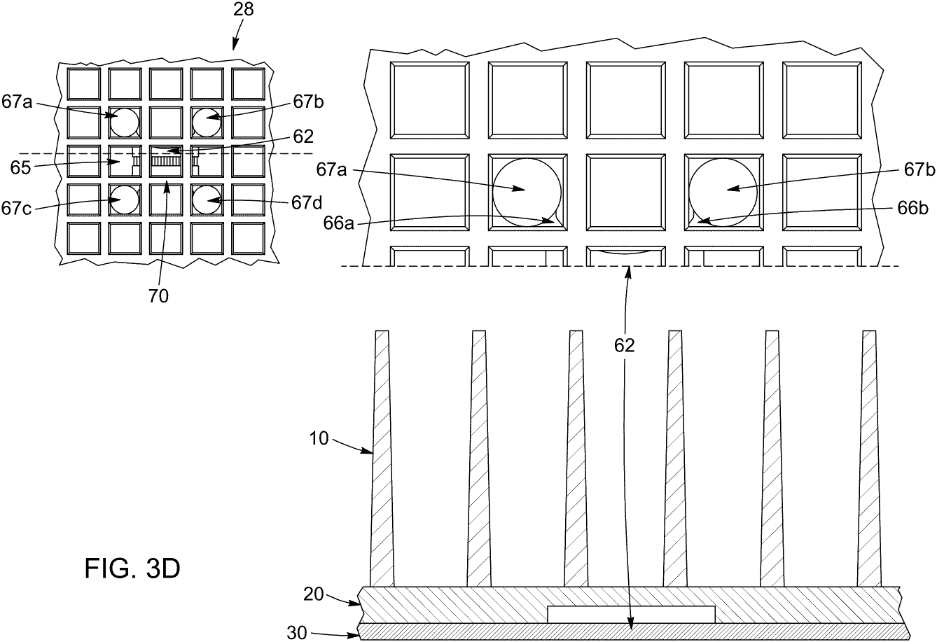

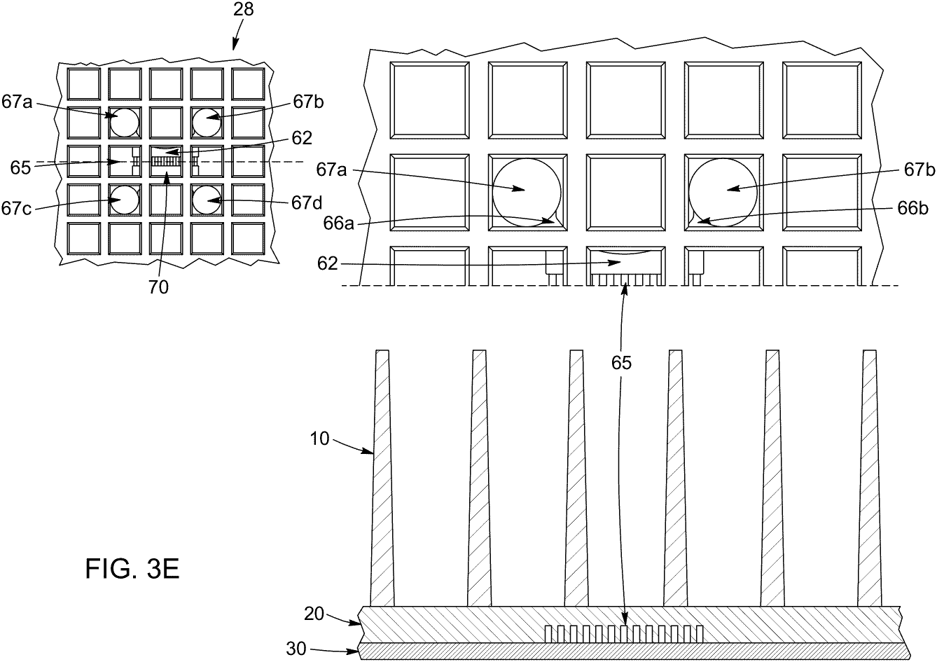

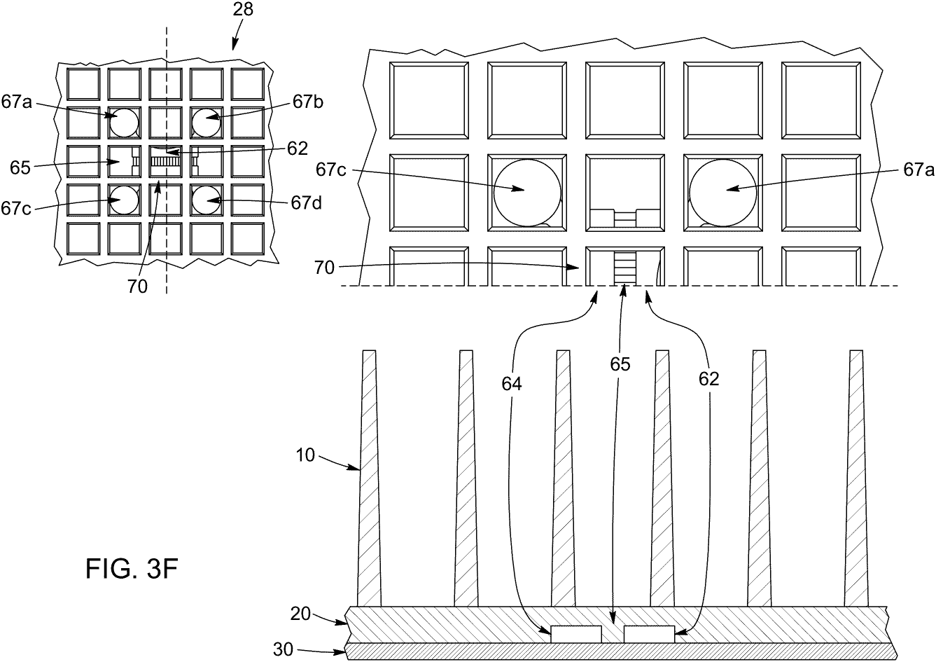

In accordance with the present technology, cells can be cultured and/or analyzed in the microfluidic layer 20. As shown in FIGS. 2A-2C and FIGS. 3A-3C, the microfluidic layer 20 is adapted for HTS and/or HCA, and comprises an upper surface 25, a bottom surface 26 and a plurality of microfluidic units 28 which can enable the analysis of cells. For instance, in FIGS. 2, the microfluidic layer 20 comprises 24 (6 × 4) microfluidic units 28.

In the implementation shown in FIG. 3A, each of the microfluidic unit 28 comprises a central main chamber 60, which in turn comprises a first compartment 62 and a second compartment 64 that each extend substantially longitudinally. In the illustrated implementation, the first and second compartments 62, 64 are substantially parallel, and they are separated by a plurality of microfluidic channels 65 that extend substantially perpendicularly between the first and second compartments 62, 64. The microfluidic channels 65 are configured to provide a fluidic communication between the first and second compartments 62, 64. In some implementations, the microchannels 65 are dimensioned so as to enable neuronal growth along the first and second compartment 62, 64, where neuronal cell bodies remain in one compartment, while axons extent along microchannels 65 to the other compartment. In some implementations, the microchannels 65 can be dimensioned to have an aspect ratio ranging from 1:1 (Width: Height) to 1:50 (Width: Height).

In the implementation shown in FIGS. 3A-3F, the microfluidic unit 28 comprises a first inlet (67a or 67b) and a first outlet (67a or 67b) disposed diagonally at opposite ends of the first compartment 62 of the central main chamber 60. In this implementation, the microfluidic unit 28 also comprises two diagonally extending microfluidic arms 66a, 66b extending diagonally in opposite directions from the first compartment 62 of the central main chamber 60. The microfluidic arms 66a, 66b provide a fluidic communication of the first inlet (67a or 67b) and the first outlet (67a or 67b) with the first compartment 62 of the central main chamber 60.

In some implementations, the microfluidic unit 28 further comprises a second inlet (67c or 67d) and a second outlet (67c or 67d) disposed diagonally at opposite ends of the second compartment 64 of the central main chamber 60. The inlets and outlets described above can enable addition and removal of liquids, particles, and cells in and from the microfluidic unit, respectively. The microfluidic unit 28 further comprises two diagonally extending microfluidic arms (66c, 66d) extending diagonally in opposite directions from the second compartment 64 of the central main chamber 60. The microfluidic arms 66c, 66d provide a fluidic communication of the second inlet (67c or 67d) and the second outlet (67c or 67d) with the second compartment 64 of the central main chamber 64. The second inlet (67c or 67d) and second outlet (67c or 67d) and connecting arms (66c, 66d) are preferable, but optional since the device could comprise the second compartment 64 without these.

In some implementations, the components of the microfluidic units 28, i.e., the inlet(s), the outlet(s), the central chamber, the microfluidic arms, the microchannels, etc., can be carved or moulded into the microfluidic layer 20.

Accordingly, in some implementations, the microfluidic layer 20 may be made of any suitable polymeric material, or other type of material, into which it is possible to carve or mould the components of the microfluidic units 28.

Alternatively, the components of the microfluidic units can be obtained by any other suitable method. For instance, the components of the microfluidic units can be obtained by 3D printing, or can be embossed in the microfluidic layer 20. Any type of material that can enable obtaining the components of the microfluidic units is suitable.

In the implementation shown in FIG. 3A, the inlets and outlets 67a, 67b, 67c, 67d are shown as a bore extending from the upper surface 25 to the lower surface 26 of the microfluidic layer 20, with the rest of the components extending from the bottom surface 26 of the microfluidic layer 20 toward the upper surface 25 of the microfluidic layer 20. The inlets and outlets 67a, 67b, 67c, 67d are also configured to be vertically aligned with corresponding wells of the multi-well grid layer 10.

In some implementations, the microfluidic layer 20 can be made of a polymeric material that is transparent to light to facilitate optical analysis and visualization of cells present in the microfluidic unit 28. Examples of materials that could be used include, but are not restricted to, polystyrene (PS), cyclo-olefin-copolymer (COC), cycloolefin polymer (COP), polymethyl methacrylate (PMMA), polycarbonate (PC), polyethylene (PE), polyethylene terephthalate (PET), polyamide (Nylon®), polypropylene (PP), polyether ether ketone (PEEK), Teflon®, polydimethylsiloxane (PDMS), and/or thermoset polyester (TPE).

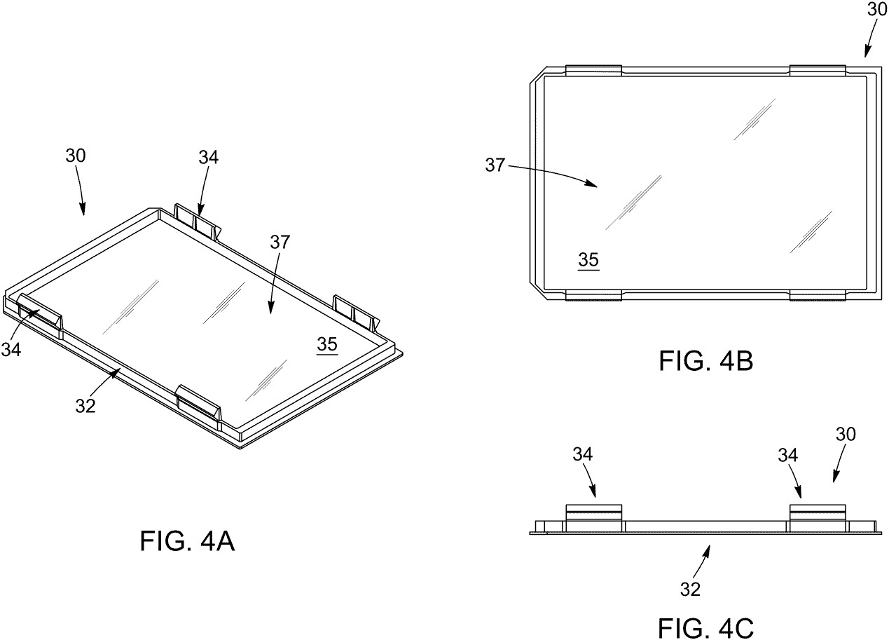

As mentioned hereinbefore and in the implementation shown in FIG. 4, the base layer 30 can be configured for being positioned beneath the microfluidic layer 20 and can be adapted for being detachably connected, or assembled, to the microfluidic layer 20 and/or to the multi-well grid layer 10. In the implementation shown in FIG. 4, the base layer 30 comprises a transparent layer 35, or translucent layer, having an upper surface 37, the transparent layer 35 being surrounded by a rectangular frame 32 comprising a plurality of hooks 34 that can be used for detachably connecting the base layer 30 to the multi-well grid layer 10. In some implementations, the transparent layer (35) is a transparent sheet that is bounded to the rectangular frame 32. In the illustrated implementation, the transparent layer 35 is integral to the rectangular frame (32), i.e., forms i.e., a single piece.

In the illustrated implementations, the base layer 30 and the microfluidic layer 20 are separate pieces since this is generally more convenient, for instance to coat the upper surface 37 of the transparent layer 35 prior to cell culture and/or to have easily access to cells attached to the transparent layer 35 once the device is disassembled and the microfluidic layer 20 is removed. Alternatively, the microfluidic layer 20 can be integral to (e.g., moulded together in a single piece) the base layer 30. The microfluidic layer 20 can also be reversibly or irreversibly attached to the base layer 30 by using any suitable method or technique, including but not limited to, compression, surface adhesion, ultrasonic welding, laser welding, thermocompression bonding, plasma bonding, solvent-assisted bonding, laser-assisted bonding, or adhesive bonding using glue, pressure sensitive adhesives, or double-sided adhesive tape.

Once the multi-well device is assembled, the bottom surface 26 of the microfluidic layer 20 can be considered to be in close contact with the upper surface 37 of the transparent layer 35. As shown in FIGS. 3A-3C and FIG. 5, the upper surface 37 of the transparent layer 35 thus provides a “floor” or a “bottom” to the inlet(s), outlet(s), central chamber, arms, and microchannels that are carved, printed, embossed and/or moulded into the microfluidic layer 20, such that fluid and suspended cells can flow in a substantially leak-tight manner from the inlet (67a or 67b) the to the outlet (67a or 67b) of the microfluidic layer 20, via the microfluidic arms (66a, 66b) and first compartment 62 of the central main chamber 60. Fluid and suspended cells can also flow in a substantially leak-tight manner from the inlet (67c or 67d) to the outlet (67c or 67d) via the microfluidic arms (66c, 66d) and second compartment 64 central main chamber 60. Fluid and suspended cells can also flow in a substantially leak-tight manner between the first compartment 62 and second compartment 64 via the microchannels 65. Likewise, the inlets and outlets (67a, 67b, 67c, 67d) can be configured to be vertically aligned with corresponding wells 12 in the multi-well grid layer 10. Therefore, once assembled, the multi-well grid layer 10, the microfluidic layer 20, the base layer 30 form at least one, preferably a plurality, of microchannel networks 24 into which a fluid and cells can flow in a substantially leak-tight manner from at least one well 12 to at least another well 12, such as shown in FIG. 5. In the illustrated implementations, the microchannel network 24 includes the combination of two wells 12, at least one microfluidic unit 28 and the upper surface 37 of the transparent layer 35.

In the illustrated implementations, the multi-well grid layer 10 comprises a hollow rectangular frame 14 surrounding a central portion comprising a plurality of wells 12 (e.g., a 384-well plates in the figures), and the rectangular frame 32 comprises a plurality of hooks 34 that are adapted to slide and snap fit into corresponding slots provided in the hollow frame 14, the hooks 34 being adapted for detachably connecting the base layer 30 to the multi-well grid layer 10. Nevertheless, the technology is not so limited since those skilled in the art can readily identify many other different means for detachably connecting, or assembling, the base layer 30 to the multi-well grid layer 10, including, but not limited to, a screw, a snap-fit, a pressure fit, a latch, a lock, a magnet, etc. Thus, any means that can enable holding, detachably connecting, or assembling, two or more parts together can be suitable. Likewise, it may be possible to connect the base layer 30 not only to the frame 14 of the multi-well grid layer 10, but also to another portion of the multi-well grid layer 10, such as the central portion and/or the wells 12 of the multi-well grid layer 10. In some implementations, the hooks can be provided inside the base layer 30 to lock or latch onto the wells of the multi-well grid layer 10.

The base layer 30 can also be connected to the microfluidic layer 20 instead of, or in addition to, being connected to the multi-well grid layer 10. For instance, the base layer 30 can be glass adhering to a microfluidic layer made of a polymer. Any type of configuration that can enable the upper surface 37 of the base layer 30 to be sufficiently close to and/or adhered to the bottom surface 26 of the microfluidic layer 20 to provide a substantially leak-tight flow of liquid and/or cells into the microchannel network 24 can be suitable.

In some implementations, the base layer 30, and more particularly any area of the base layer 30 that is in contact with the microfluidic layer 20, can be composed of a polymeric material that is transparent to light (e.g., glass, polymers, thermoplastic) in order to provide for optical analysis and visualization of cells into the central compartment 60 of the microfluidic layer (20). Examples of materials that could be used include, but are not restricted to, polystyrene (PS), cyclo-olefin-copolymer (COC), cycloolefin polymer (COP), polymethyl methacrylate (PMMA), polycarbonate (PC), polyethylene (PE), polyethylene terephthalate (PET), polyamide (Nylon®), polypropylene or polyether ether ketone (PEEK), Teflon®, polydimethylsiloxane (PDMS), and/or thermoset polyester (TPE).

In some implementations, the base layer 30 is made of a transparent sheet (e.g., transparent base layer or bottom) bounded to and surrounded by the rectangular frame 32. In some implementations, the transparent base layer 35 and the rectangular frame 32 are integral and fabricated as a single part.

Advantageously, the upper surface 37 of base layer 30 can be coated with a substance or compound, for instance a substance that promotes cellular adhesion, that promotes cellular growth or repel cellular adhesion. Possibly useful coating substances include, but are not limited to, poly-I-lysine (PLL), poly-d-lysine (PDL), poly-L-ornithine (PLO), collagen, laminin, Matrigel®, and bovine serum albumin. In some cases, the upper surface 37 of the base layer 30 can be chemically modified with one or more of poly [carboxybetaine methacrylate] (PCBMA), poly [2-methacryloyloxy) ethyl-trimethylammonium chloride] (PMETAC), poly [poly(ethylene glycol) methyl ether methacrylate] (PPEGMA), poly [2-hydroxyethyl methacrylate] (PHEMA),poly[3-sulfopropyl methacrylate] (PSPMA), and poly [2-(methacryloyloxy)ethyl dimethyl-(3- sulfopropyl) ammonium hydroxide] (PMEDSAH). In some implementations, the base layer 30 can be treated with plasma, and/or with chemicals.

As shown in FIGS. 6A-6B, the multi-well device may also comprise a lid 40. In some implementations, the lid 40 can be transparent. In some implementations, the lid 40 comprises a transparent horizontal layer 44 surrounded by a rectangular frame 42. The lid 40 can be dimensioned to fit over the multi-well grid layer 10, such that the rectangular frame 42 of the lid 40 can at least partially surround the rectangular frame 14 of the multi-well grid layer 10.

FIGS. 6C-6D show the top and bottom portion of an assembled multi-well device that includes a lid. As shown in FIG. 6C from the top, one can visualize the wells 12 (i.e., 384 wells in the illustrated implementation) of the multi-well grid layer 10, and inlets/outlets 67 of a microfluidic layer 20. As shown in FIG. 6D, from the bottom, one can visualize the microchannel networks 24 (a total of 24 in the illustrated implementation) through the transparent layer 35 of the base layer 30.

As mentioned hereinbefore, the components of the multi-well device described herein can be produced from a wide range of materials, including thermoplastics. In some implementations, the transparent layer 35 can be made of glass, whereas the other layers are made of thermoplastics or silicone polymer material. However, to meet cost criteria for high-throughput screening where disposable substrates may be preferred, and large volumes of devices may be consumed, the components may be produced from PS, COC or COP, as these materials may be amenable to cost-efficient high-volume production methods such as 3D printing, injection moulding, hot embossing, or computer-aided manufacturing (CAM) micro machining.

In some implementations, the material to be used is amenable for surface coatings to enable culture of cells. For example, it may be desirable in some implementations to carry out physical surface treatments, e.g., plasma treatment or corona discharge, as well as to coat the substrate with materials proteins or polymeric materials such as poly-l-lysine, poly-L-ornithine, collagen, laminin, Matrigel®, bovine serum albumin or other protein solutions. Furthermore, chemical modifications can also be grafted onto the surface, using for instance at least one of poly [carboxybetaine methacrylate] (PCBMA), poly [2-methacryloyloxy) ethyl] trimethylammonium chloride] (PMETAC), poly [poly (ethylene glycol) methyl ether methacrylate] (PPEGMA), poly[2-hydroxyethyl methacrylate] (PHEMA), poly[3-sulfopropyl methacrylate] (PSPMA), and poly[2-(methacryloyloxy)ethyl dimethyl-(3-sulfopropyl)ammonium hydroxide] (PMEDSAH).

### Kits

Advantageously, the multi-well device of the present technology can be sold in kits to be assembled. For instance, a kit according to the present technology can comprise a combination of two or more of (i) a multi-well grid layer as defined herein (ii) a microfluidic layer as defined herein, and (iii) a base layer as defined herein. In implementations the kit comprises a first piece comprising: (1) a microfluidic layer bounded or integral (i.e., a single piece) to the multi-well grid layer as defined herein; and (2) a base layer (e.g., a transparent layer bounded or integral to a frame as defined herein above). The kit may also comprise additional elements, including but not limited to, a lid, additional layers, coating substance(s), operating instructions, buffer(s), cell culture media, cells, etc.

### Configurations of the Multi-Well Device

Advantageously, the multi-well device described herein can be configured for using commercially available bottomless multi-well plate comprising 6, 12, 24, 48, 96, 384, or 1536 wells. The multi-well device described herein can also be configured to comprise a plurality of microfluidic units having different sizes and configurations.

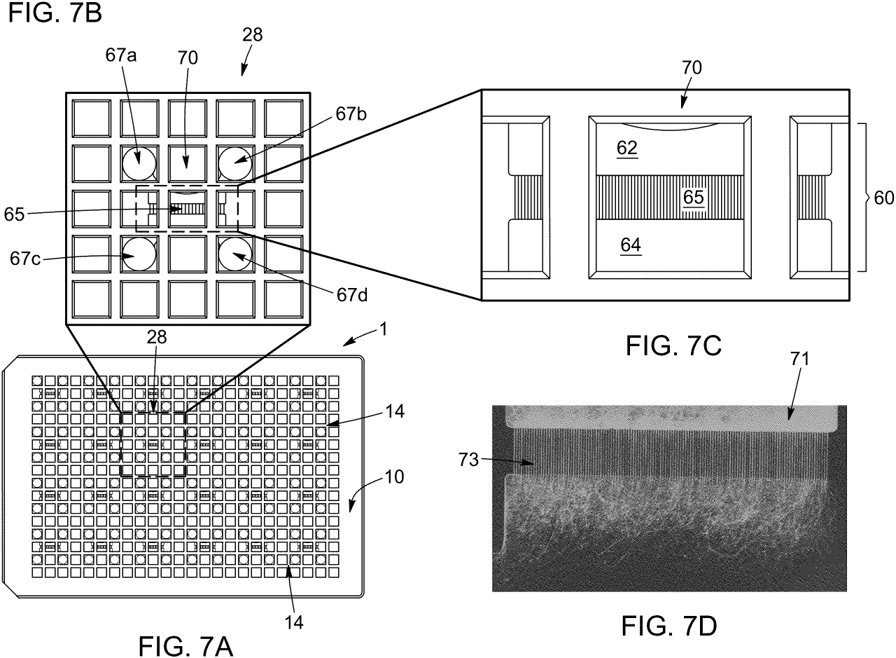

FIGS. 7A-7B illustrate one particular example of a configuration of a multi-well device that includes a 384-well plate as the multi-well grid layer 10. In this configuration, 24 microfluidic units (dark square) are disposed underneath the multi-well grid layer 10, i.e., underneath the 384-well plate (FIG. 7A). In this configuration, each microfluidic unit 28 comprises two pairs of inlets-outlet (67a-67b and 67c-67d), and one central compartment 60 occupying a total space corresponding to nine 9 wells (i.e., 3 × 3 arrangement) of the 384-well plate. In this 3 × 3 arrangement, the centre well defines a central assay window 70, while the inlets and outlets align with four opposite corner wells (FIG. 7B). A zoom of the visualization window shows microchannels 65 (FIG. 7C), the microchannels 65 enabling a precise organization of cultured cells. FIG. 7D shows an example of rat cortical neurons labelled with beta-tubulin antibody where the neuronal cell bodies 71 stay in the first compartment 62, while the axons 73 extend within the microchannels 65 to the second compartment 64.

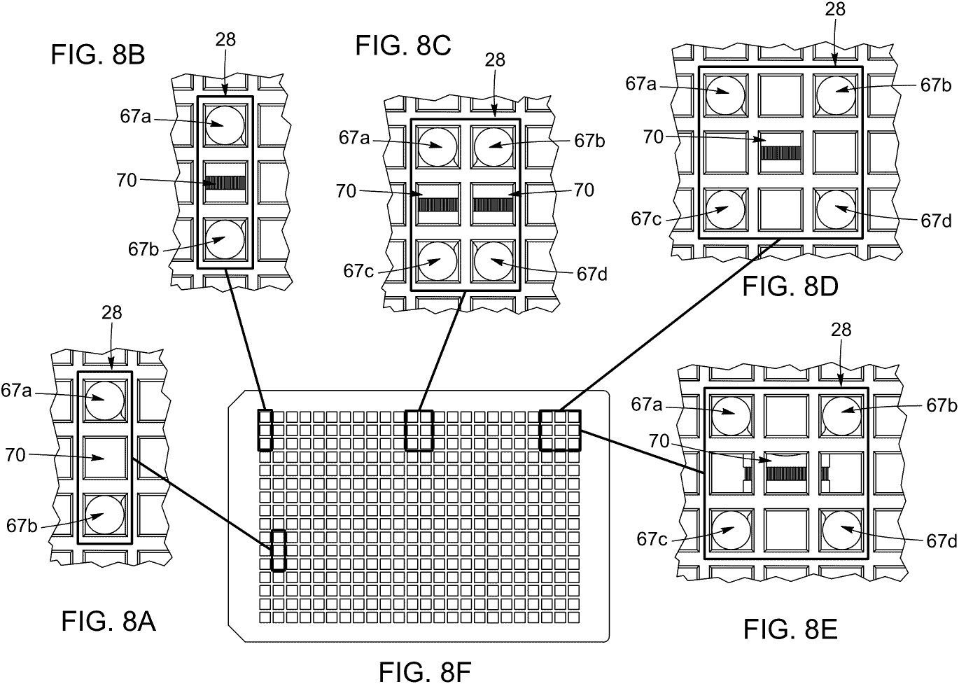

FIG. 8 illustrates various examples of additional configurations of microfluidic units that can be provided under a 384-well plate as the multi-well grid layer 10. As illustrated, the present technology can be adapted such that:
the microfluidic unit 28 occupies a space corresponding to three (3) wells in a 1 × 3 arrangements with one centre well being aligned over one single central chamber (i.e., no separating microchannels), the centre well defining an assay window 70;
the microfluidic unit 28 occupies a space corresponding to three (3) wells in a 1 × 3 arrangements, with one centre well being aligned over two compartments (i.e., a central chamber having a first and second compartments, with microchannels extending therebetween), the centre well defining an assay window 70;
the microfluidic unit 28 occupies a space corresponding to six (6) wells in a 2 × 3 arrangement with two centre wells, each centre well being aligned over a corresponding central chamber, with each central chamber having a first and second compartments with microchannels extending therebetween, the two centre wells defining two separate assay windows 70;
the microfluidic unit 28 occupies a space corresponding to nine (9) wells in a 3 × 3 arrangement with one centre well aligned over a central chamber having a first and second compartments, with microchannels extending therebetween, the centre well defining an assay window 70;
the microfluidic unit 28 occupies a space corresponding to nine (9) wells in a 3 × 3 arrangement with three centre wells being aligned over a central chamber having a first and second compartments with microchannels extending therebetween, the centre well defining an assay window 70.

In implementations, the assay window 70 can be formed between two or more wells. In some implementations, the assay window 70 comprises a height of visualization from 0.001 mm to 5 mm. In implementations, the assay window 70 comprises one or multiple sections (e.g., the assay window 70 is defined by one or multiple wells). In some implementations, the assay window 70 comprises one or multiple series of microchannels 65. In some implementations, the assay window 70 comprises two or more sections connected by one or multiple series of microchannels 65.

The configuration, length and dimensions of each microfluidic unit 28 can be adapted to particular needs. For instance, it is possible to adjust the width and height of the central main chamber 60 and the width and height of the first compartment 62, the second compartment 64 and of the microchannels 65, the angle, width, length of the microfluidic arms 66, the diameter and height of the inlets and outlets 67, etc.

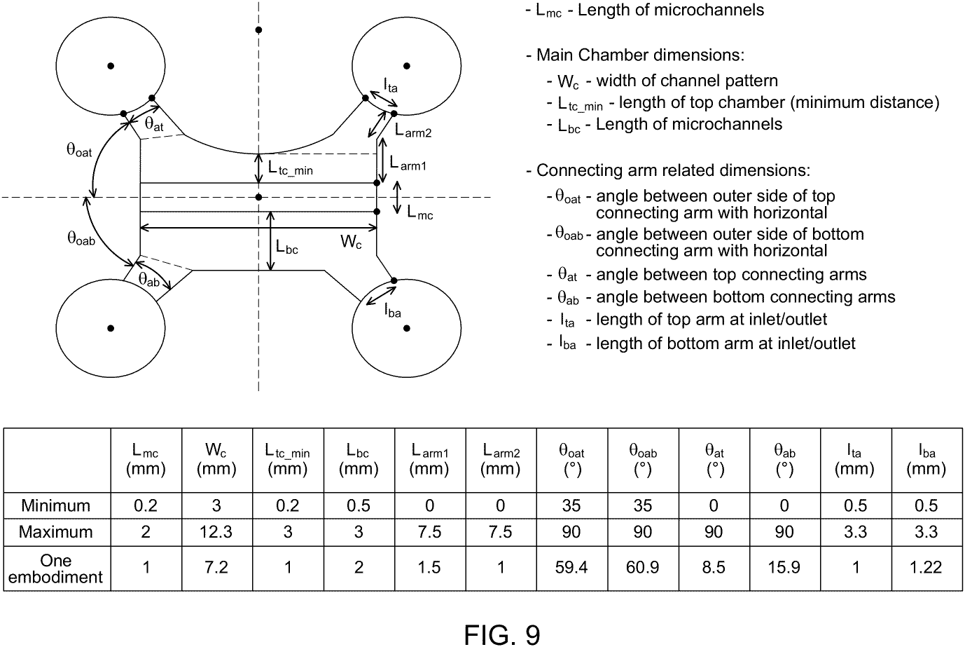

FIG. 9 provides examples of ranges of dimensions of various components of the microfluidic unit 28 that can be implemented when using microfluidic units with a 24-well plate, a 96-well plate or a 384-well plate as the multi-well grid layer 10. Different configurations are possible to increase or reduce the number of microfluidic units per plate, as well as to adapt the dimensions of the microfluidic units to fit with 6, 12, 24, 48, 96, 384 or 1536-well plates. It is to be noted that the range of values listed in FIG. 9 are per parameter, and not to be taken in combination. The third row provides values for parameters in accordance with one example implementation.

Those are only selected examples of the many possible configurations that can be envisioned according to the present technology and each single microfluidic layer can be adapted to comprise a plurality of identical configurations or it may be adapted to comprise many different configurations of microfluidic units, as illustrated in FIG. 8.

It is to be understood that when referring to a microfluidic layer herein, the microfluidic layer can be a single layer of material that includes given microfluidic units, or the microfluidic layer can include a plurality of layers to achieve given microfluidic units.

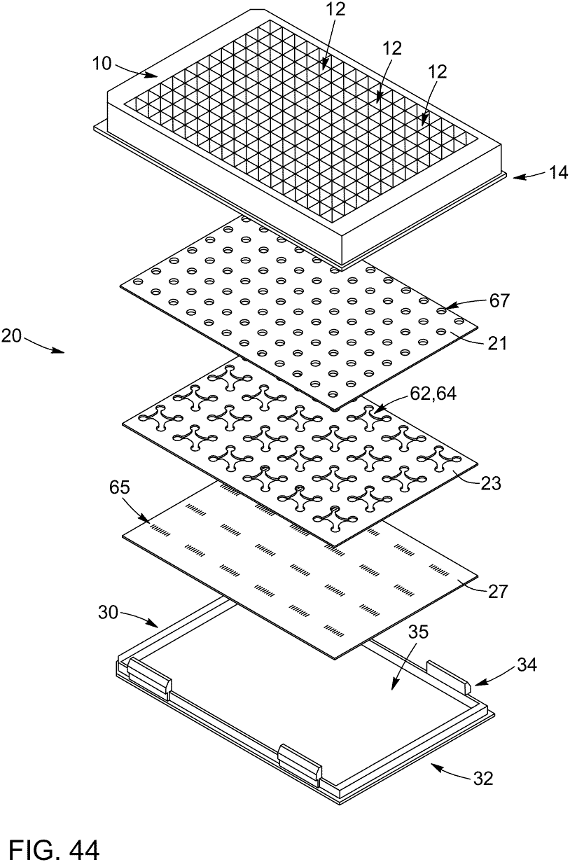

FIG. 44 illustrates an example of a microfluidic layer 20 that successively includes a first layer 21, a second layer 23 and a third layer 27. The first layer 21, the second layer 23 and the third layer 27 together form the microfluidic layer 20. In the implementation illustrated in FIG. 44, the configuration of each microfluidic unit is similar to the microfluidic units described above with reference to FIG. 1. The first layer 21 includes the inlets and outlets 67 of the microfluidic unit, the second layer 23 includes the first and second compartments 62, 64, and the third layer 27 includes the microchannels 65. Once the first layer 21, the second layer 23 and the third layer 27 are superposed to each other, the resulting microfluidic layer 20 includes microfluidic units that each includes inlets and outlets 67, first and second compartments 62, 64, and microchannels 65.

Although the microfluidic layer 20 shown in FIG. 44 includes three layers, it is to be understood that in other implementations, the microfluidic layer 20 can include two layers to achieve desired microfluidic units, or can include more than three layers to achieve desired microfluidic units.

Furthermore, although the microfluidic layer, being provided as a single layer or including a plurality of layers, has been described in relation with the figures as including microfluidic units, it is to be understood that the microfluidic layer can be configured to include any type of pattern that can form a patterned unit that can facilitate culturing cells as part of the multi-well device described herein. Thus, in some implementations, the microchannels can be omitted and optionally be replaced by macrochannels, channels can be omitted altogether, the first and second compartments can have various shapes and sizes, etc. In some implementations, the patterned unit can include inlets and outlets only.

Various options are possible to provide certain components of the multi-well device integral to each other or as distinct features of the multi-well device. For instance, in some implementations, the multi-well grid layer 10 can be integral with the first and second layers 21, 23 shown in FIG. 44, and the third layer 27 shown in FIG. 44 can be integral with the base layer 30. In other implementations, the first, second and third layers 21, 23, 27 shown in FIG. 44 can be integral with the base layer 30. In yet other implementations, the first, second and third layers 21, 23, 27 shown in FIG. 44 can be integral with each other, with the multi-well grid layer 10 and the base layer 30 being provided as separate components.

In some implementations, the microfluidic layer 20 can include a drug administration port to enable real-time drug administration. In some implementations, when a drug administration port is integrated in the microfluidic layer, real time drug administration and recordings from cells can be performed simultaneously.

In some implementations, multi-well device can be used with a dedicated plate reader that is configured to enable control of variables related to cell culture, including the temperature and humidity at which the cells are cultured, for instance. This type of dedicated plate reader can be advantageous for instance when performing cell cultures experiments that spans over a few days, or to perform long term experiments. In addition, the plate reader can be operatively connected to a processor, which can optionally be equipped with a custom software, for real time observations and analysis of the recordings.

### Culture Methods and Cell Analysis

Additional aspects of the technology concerns methods for cell culture and methods for cell analysis.

In some implementations, the method for cell culture and/or cell analysis comprises: (1) providing a microfluidic assembly comprising a multi-well grid layer 10 and a microfluidic layer 20 as defined herein; (2) connecting a base layer 30 as defined herein to the microfluidic assembly to obtain a multi-well device as described herein; (3) loading cells to be analyzed in a well (e.g., inlet); and (4) analyzing loaded cells (e.g., cells in the assay window).

In some implementations, the analysis of cells comprises optical analysis (optical microscopy, fluorescence microscopy, etc.). The present technology may also be amenable to other types of analysis, including but not limited to electrical stimulation, absorbance, spectrophotometry, mass spectroscopy, electrical impedance, etc.

In some implementations, the multi-well device can be used to measure neurite length, neurite morphology, network formation, neurite branching, synapse formation, neurite thickness using manual analysis and measurement methods or digital analysis and measurements with specific application notes and software to automate measurements of neuronal morphology, health and activity.

In some implementations, the multi-well device can also be used to enable selective measurement of the effect of compounds on neurites or on soma with neurites, including the collection of medium for biochemical measurements from the neuronal or soma compartments.

In some implementation, the analysis of the cells can be carried out in the central main chamber of the microchannel network.

In implementations, the multi-well grid layer of the microfluidic assembly comprises at least 6, 12, 24, 48, 96, 384, 1536 or 3456 wells and the microfluidic assembly is adapted for high throughput optical analysis.

In implementations, the method further comprises coating the base layer prior to loading cells. In implementations, the method further comprises culturing the loaded cells for a certain period of time (e.g., 1 h, 2 h, 12 h, 24 h, 48 h, 7 days, 14 days, 30 days or more) prior to analysis. In implementations, after the culturing the cells, the method further comprises detaching the microfluidic assembly from the base layer, leaving organized cells attached to the base layer, wherein these attached cells can further be cultured and/or analyzed.

### Advantages

Advantageously, and as illustrated in the examples hereinafter, the multi-well device described herein can provide numerous benefits for cell culture and cell analysis.

One of the advantages is to provide a “microscopic window” with a precise architecture to organize cells in vitro with similar structure as in vivo in a microplate compatible with HTS and HCA automation equipment. This can find numerous applications in the fields of parasitology, fertility (e.g., analysis sperm, such as analysis of individual spermatozoa), cancer, muscular diseases, immune diseases, Alzheimer’s disease (e.g., neuronal cells), etc. The present technology is also compatible with standard plate readers and microscopes.

The present technology can also enable the creation of in vitro models compatible with human and rodent neurons derived from the central nervous system (CNS) and peripheral nervous system (PNS). Indeed, the technology can enable reproduction of the precise in vivo organization of cells in an in vitro environment while being easily scalable to enable high capacity experimentation such as HTS and HCA of thousands to hundreds of thousands of compounds. For instance, in accordance with some implementations and as shown in the examples, the technology can provide for a high throughput analysis of neuronal morphology, network organization and connectivity with over 2,400 axons/plate. It can also provide for high-throughput screening of compound toxicity and efficacy on neurons. It can also enable for 2D cell culture as well as 3D cell culture. It can also enable co-culture of neurons with one or more cell types (e.g., astrocytes, oligodendrocytes, keratinocytes, myocytes, etc.). The co-culture can be used to promote innervation of 3D tissues and/or to study myelination, neuro-immune interactions, innervation, etc. Accordingly, the multi-well device described herein can thus support multiple applications such as neurotoxicity tests, disease modelling, axonal transport, drug screening, neurodevelopmental studies, mechanism of action of compounds, intracellular and extracellular events, network formation, etc. The present technology is versatile and compatible with cultures of mammalian neurons derived from the central and peripheral nervous system. Precise dimensions of the compartments can guide an efficient fluidic flow to promote substantially uniform distribution of nutrients and to promote neuronal survival, for instance for over 5 weeks.

The multi-well device described herein comprises an architecture that can enable control of seeding density of any cell, and can significantly reduce the number of cells and reagents that are being used, compared to many existing cell culture devices.

As the multi-well device described herein comprises a base layer that can be removed, the upper surface of the base layer can be treated, i.e., the surface where the cells will attach and grow, by specific methods or techniques (e.g., plasma treatment) or by coating with any desired molecule or substance. Moreover, as the cells usually adhere to the upper surface of the base layer, i.e., the surface that can be treated, the multi-well device can be disassembled to perform additional tests, such as network formation and connectivity.

The present technology also provides means for increasing reproducibility, for enabling automation with HTS/HCA, for accelerating testing of drug candidates and reducing costs.

The present technology can find useful applications in many different industries including, but not limited to, pharmaceutical industry, cosmetics industry, food industry environmental industry, etc. For instance, specific applications for the pharmaceutical field may include drug screening, drug repurposing, toxicity testing, disease modelling, etc.

Those skilled in the art will recognize, or be able to ascertain, using no more than routine experimentation, numerous equivalents to the specific procedures, implementations, claims, and examples described herein. Such equivalents are considered to be within the scope of this technology, and covered by the claims appended hereto. The technology is further illustrated by the following examples, which should not be construed as further or specifically limiting.

### Implementations Related to the Combination of a Microfluidic Layer With an Electrode Layer

In some implementations, the multi-well device as described herein can further be configured to receive therein or in proximity thereof an electrode or a group of electrodes such that the electrode or group of electrodes can be in contact, either direct or indirect, i.e., in electrical communication, with the cells that are cultured in the microfluidic layer 20. When the cells cultured in the microfluidic layer 20 are neuronal cells, the electrode or group of electrodes can be used for instance to analyze neuronal electrical activity.

The electrode or the group of electrodes can be distributed over an electrode layer such that the positioning of the electrode or group of electrodes can be determined at least in part according to the architecture of the microchannels 65 of the microfluidic units 28 of the microfluidic layer 20. Conversely, the architecture of the microchannels of the microfluidic units 28 of the microfluidic layer 20 can be determined at least in part according to the positioning of the electrode or the group of electrodes over the electrode layer. The positioning of the electrodes in relation to the microchannels 65 can enable aligning the microchannels 65 with the electrodes, which in turn, can contribute to improving the interaction between the cells present in the microchannels and the electrodes. The microfluidic pattern, i.e., the architecture of the microchannels, enables adhesion and organized growth of the cells on top of or underneath electrodes or in proximity to the electrodes, which in turn can contribute to improving electrode detection of signals from cells as well as improving precision and accuracy of detected signals. The microfluidic pattern enabling the positioning cells on top of, underneath or in proximity to electrodes can also facilitate precise sensing, or detection, of signals from cells, and/or stimulation of cells, thereby increasing accuracy of electrode readings.

Accordingly, the distribution of the electrodes over the surface area of the electrode layer can be such that it enables aligning the microchannels of the microfluidic unit with the electrodes to achieve a predetermined organized architecture of the microchannels of the microfluidic layer relative to the electrodes, instead of the electrodes being provided randomly relative to the architecture of the channels of the microfluidic layer. Providing a predetermined organized architecture of the microchannels of the microfluidic layer relative to the electrodes can improve the accuracy of the stimulation by the electrodes and/or detection of signals by the electrodes, since with a predetermined organized architecture of the microchannels of the microfluidic layer relative to the electrodes, it is possible to identify a given location, e.g., a given microchannel of the microchannels, of a signal detection and to target a given location, e.g., a given microchannel of the microchannels, for cell stimulation.

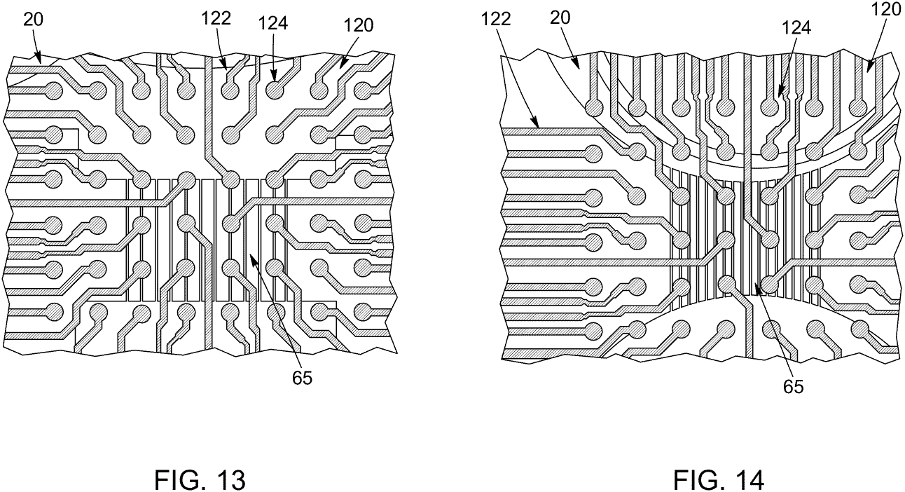

FIGS. 13 and 14 illustrate examples of a microfluidic layer 20 combined with an electrode layer 120 that includes a group of electrodes 122, each electrode including a tip 124 serving as a measuring point. The microfluidic layer 20 includes microchannels 65 that can be configured to enable neuronal growth, with axons extending within the microchannels 65, and cells bodies remaining in adjacent compartments, for example. In FIGS. 13 and 14, the respective tip 124 of several of the electrodes 122 of the electrode layer 120 is shown as being aligned with a given microchannel of the microchannels 65. In the context of the present description, the term “aligned” is intended to mean that there is a controlled organization of the electrodes with respect to the microchannels. For instance, when axons are present in the microchannels, such controlled organization of the electrodes with respect to the microchannels can enable the electrodes to interact with an organized architecture of the axons growing in the microchannels instead of randomly distributed axons, or instead of a random interaction between microchannels and electrodes.

When using the term “interacting” herein, it is to be understood that it refers to the interaction between the cells and the electrodes, i.e., the electrical communication between the cells and the electrodes, and can include a stimulation of the cells by an electrode, or a detection of a signal from the cells by an electrode, for instance. In some implementations, the interaction of the cells with the electrodes can be enabled by a contact, direct or indirect, of the cells with the electrodes.

The term “align” can be interpreted as referring to a positioning of the microchannels of the microfluidic layer at a given position relative to the electrodes of the electrode layer or electrode grid, or to a positioning the electrodes of the electrode layer or electrode grid at a given position relative to the microchannels of the microfluidic layer. As used herein, an “electrode grid” refers to a patterned organization of a plurality of electrodes, such as shown in FIGS. 13, 14 and 16 for instance.

For instance, in some implementations, aligning the microchannels of the microfluidic unit with the electrodes of the electrode layer can include aligning at least one microchannel with a predetermined number of the electrode tips. In some implementations, aligning the microchannels of the microfluidic unit with the electrodes of the electrode layer can include aligning a predetermined number of the electrode tips laterally along the microchannels. In other implementations, aligning the microchannels of the microfluidic unit with the electrodes of the electrode layer can include positioning a predetermined number of the microchannels over a predetermined number of electrode tips. In yet other implementations, aligning the microchannels of the microfluidic unit with the electrodes of the electrode layer can include placing the microfluidic layer over the electrode layer such that the microchannels extend substantially vertically or substantially horizontally and intersect a predetermined number of electrode tips. In yet other implementations, aligning the microchannels of the microfluidic unit with the electrodes of the electrode layer can include positioning the microfluidic layer such that the microchannels intersect the electrode tips along an entire diameter of the electrode tips. In some implementations, the electrode layer can be designed to fit the microfluidic layer, or can be designed for specific types of measurements or experiments, for instance to measure any type of chemical, electrical and/or biological signal.

FIG. 15 is a prior art figure that illustrates cultured neuronal cells randomly distributed with respect to the electrodes of an electrode layer. This random distribution of the cultured neuronal cells as depicted in prior art FIG. 15 can result in a multitude of indiscernible signals from cells that may or may not be in electrical communication with the electrodes. For instance, if the electrodes illustrated in prior art FIG. 15 were used to stimulate the randomly distributed axons, a random number of axons would end up being stimulated given that a random number of axons are in electrical communication with the electrodes. Similarly, if the electrodes illustrated in prior art FIG. 15 were used to detect a response from the randomly distributed axons, a random number of axons can end up having a response detected given that a random number of axons are in electrical communication with the electrodes.

In neuronal cultures lacking an organized architecture, such as shown in prior art FIG. 15, any part of the neuron can end up being in contact with electrodes, such as cell bodies, axons, and/or dendrites. In such neuronal cultures lacking an organized architecture, the electrodes are used to detect or stimulate electrical responses from the whole cell culture and as a result, the readings that are obtained include a mixture of signals arising from an undetermined number of cells or cell parts that are in contact with the electrodes.

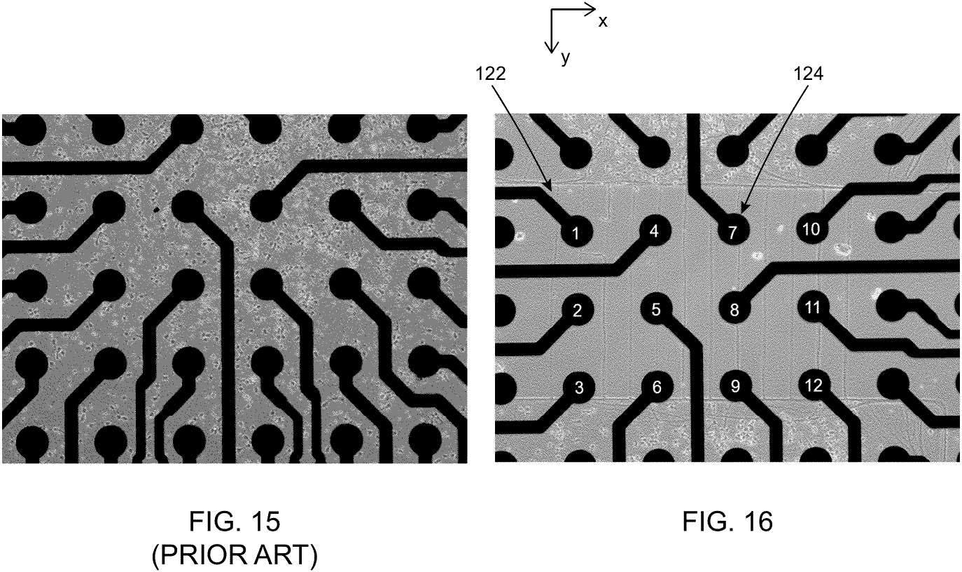

In contrast, a predetermined organized architecture of microchannels relative to electrodes can enable specific cell components of the neuronal cells to be placed in electrical communication with electrodes. For instance, FIG. 16 illustrates that the axons are placed in contact with electrodes tips #1, #2, #4, #5, #7, #8, #10 and #11.

The predetermined organized architecture of microchannels relative to electrodes can enable stimulating and sensing signals from precise cells or cell parts, it is possible to control which cells and which cell parts are in contact with the electrodes. The predetermined organized architecture of microchannels relative to electrodes also promotes higher well-to-well reproducibility because every well can have the same or similar architecture of microchannels, and it is thus possible to determine the direction of the signal path from cell to cell and how compounds can impact the signal path.

FIG. 16 illustrates neuronal cells that are grown in a microfluidic layer 20 as described herein, with axons extending within the microchannels 65 of the microfluidic layer 20. FIG. 16 illustrates axons growing in the vicinity of the electrodes according to an organized architecture, so that the presence of the axons in the vicinity of the electrodes can be predictable or controlled. The tips 124 of the electrodes that are superposed to, or placed underneath, the microfluidic layer 20 have been numbered 1-12 in FIG. 16 for ease of reference.

FIG. 16 illustrates that following alignment of the electrode layer 124 with the microchannels of the microfluidic layer, a substantially uniform distribution of the tips of the electrodes is obtained over the microfluidic channels, and thus over the axons. In the implementation illustrated in FIG. 16, each electrode tip 124 of the 12 electrode tips is provided at a substantially constant distance from each other longitudinally and laterally, i.e., along the y axis and the x axis respectively. Still in the implementation illustrated in FIG. 16, the tip of each of the electrodes is directly adjacent or intersects a series of three microfluidic channels into which axons are growing. For instance, tip #1 in the top left corner of FIG. 16 is adjacent to a first microfluidic channel on the left-hand side, intersects a second microfluidic channel, and is adjacent to a third microfluidic channel on the right-hand side. Tip #2 is longitudinally spaced-apart from tip #1, and is also adjacent to the first microfluidic channel on the left-hand side, intersects the second microfluidic channel, and is adjacent to the third microfluidic channel on the right-hand side, and so on for tip #3. Tip #4 is laterally spaced-apart from tip #1, and is adjacent to a fourth microfluidic channel on the left-hand side, intersects a fifth microfluidic channel, and is adjacent to a sixth microfluidic channel on the right-hand side, and so on. As mentioned above, this controlled distribution of the tips of the electrodes with respect to the microchannels of the microfluidic layer can facilitate controlling the location of stimulation of the axons present in the microchannels as well as the detection of signals from the axons present in the microchannels. In other words, the microfluidic layer and more particularly the microchannels of the microfluidic layer can facilitate positioning the axons at a given location such that the axons can be stimulated at this given location by the electrodes and the signal can be measured from the axons at this given location using the electrodes. In the implementation shown in FIG. 16, each series of three electrodes, i.e., tip #1, tip #2 and tip #3, tip #4, tip #5 and tip #6, tip #7, tip #8 and tip #9 and tip #10, tip #11 and tip #12, connects a first population of neurons present in the upper portion of the figure with the second population of neurons present in the below portion of the figure, the expressions “upper portion” and “below portion” being used to facilitate reference to portions of the figure. In this example, the first population of neurons were seeded in a top inlet (67a or 67b) and a bottom inlet (67c or 67d) of a microfluidic unit 28 as described herein. It is to be understood that cells can also be seeded in only one of the top and bottom inlets.

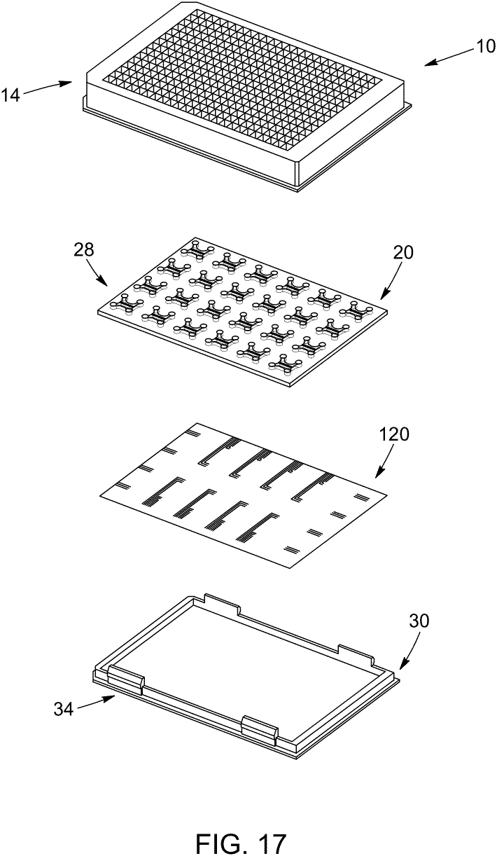

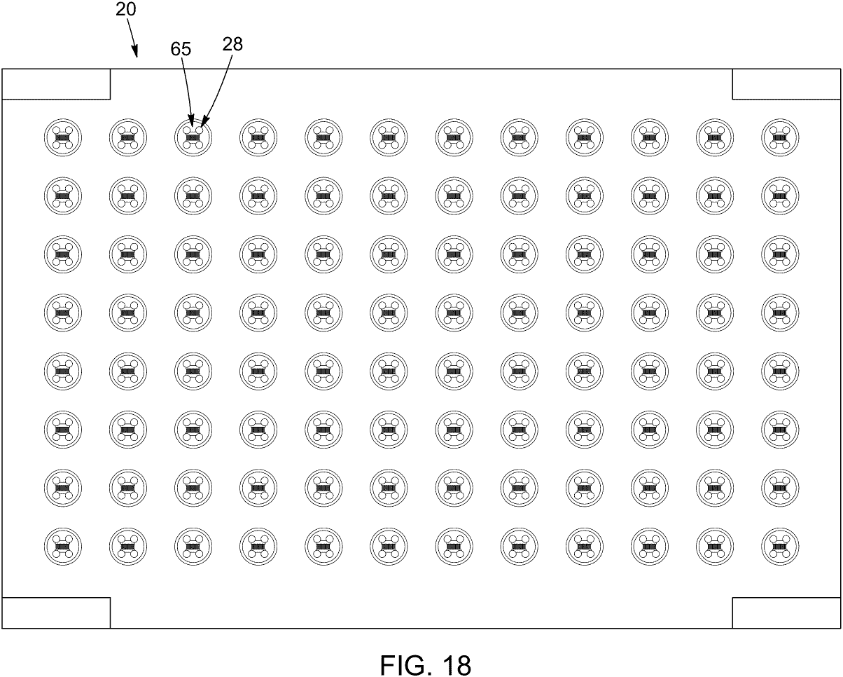

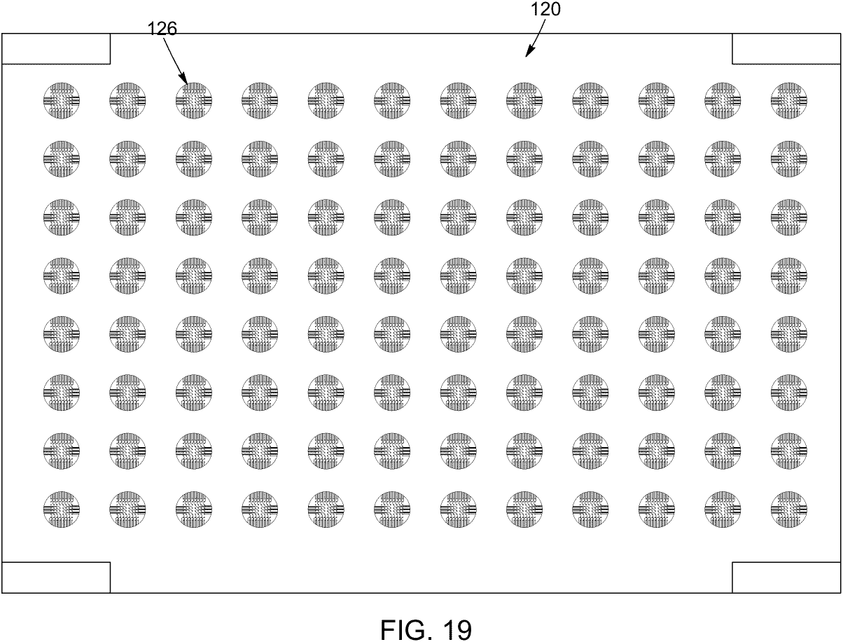

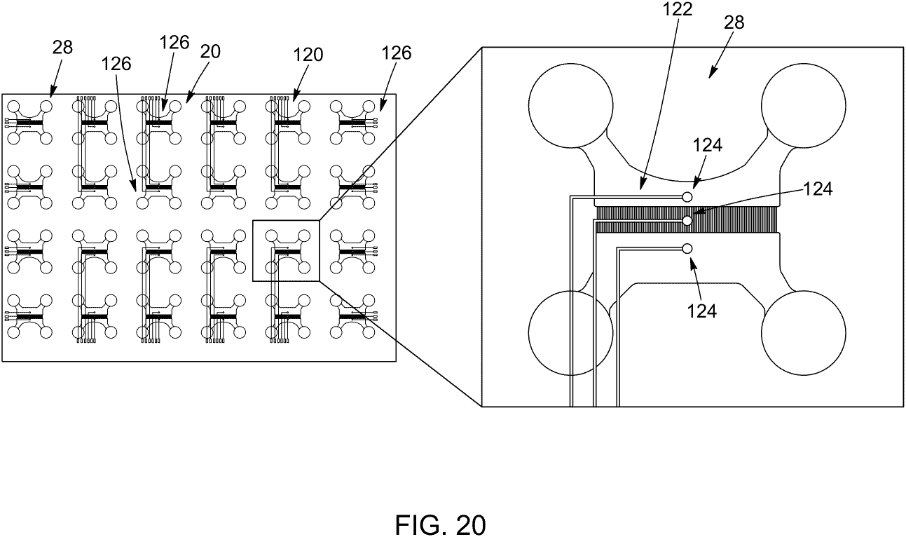

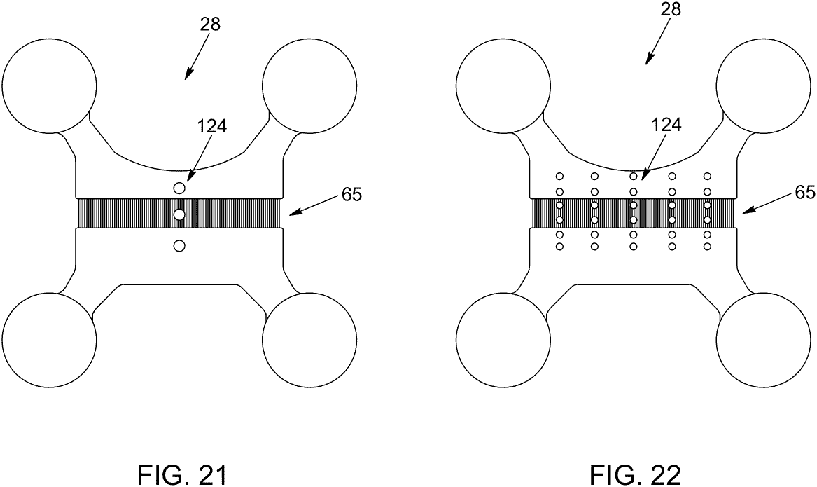

FIGS. 17-20 illustrate an implementation of an electrode layer 120 that can be used in cooperation with a microfluidic layer 20, a multi-well grid layer 10 and a base layer 30 as described herein. In FIGS. 17 and 20, the microfluidic layer 20 includes 24 microfluidic units 28, and the electrodes 122 of the electrode layer 120 are distributed to achieve a predetermined organized architecture of the microchannels relative to the electrodes. In FIG. 18, the microfluidic layer 20 includes 96 microfluidic units 28, and the electrode layer 120 shown in FIG. 19 includes a corresponding number of electrode grids 126 to achieve a predetermined organized architecture of the microchannels relative to the electrodes. As used herein, the term “grid” refers to a grouping of electrodes associated with a microfluidic unit 28. The electrode layer 120 can be placed onto the upper surface of the base layer 30 and underneath the microfluidic layer 20. In other words, the electrode layer 120 can be sandwiched between the upper surface of the base layer 30 and the microfluidic layer 20.

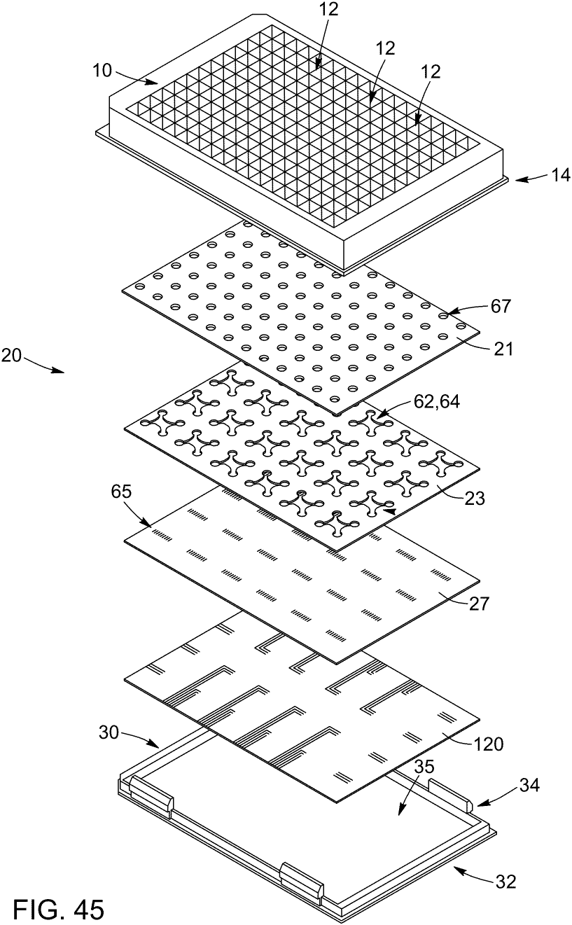

FIG. 45 illustrates an example of a microfluidic layer 20 that successively includes a first layer 21, a second layer 23 and a third layer 27 as described above with reference to FIG. 44. The first layer 21, the second layer 23 and the third layer 27 together form the microfluidic layer 20. In the implementation shown in FIG. 45, the first layer 21 includes the inlets and outlets 67 of the microfluidic units, the second layer 23 includes the first and second compartments 62, 64, and the third layer 27 includes the microchannels 65. Once the first layer 21, the second layer 23 and the third layer 27 are superposed to each other, the resulting microfluidic layer 120 includes microfluidic units that each includes inlets and outlets 67, first and second compartments 62, 64, and microchannels 65. The microfluidic layer 20 is then superposed to an electrode layer 120 as described herein, such that the electrodes of the electrode layer 120 are aligned with the microchannels 65 of the microfluidic layer 20. It is to be noted that although the electrode layer 120 shown in FIG. 45 is provided underneath the third layer 27 of the microfluidic layer 20, in alternative implementations, the electrode layer 120 can be placed at any location between the multi-well grid layer 10 and the base layer 30, such as between the first layer 21 and the second layer 23, for instance.

In some implementations, the electrode layer can be configured such that the spatial distribution of the electrodes is predetermined in accordance with the spatial distribution of the microchannels of the microfluidic layer, such that when the microfluidic layer and the electrode layer are placed in contact, a predetermined organized architecture of the microchannels relative to the electrodes can be achieved. In some implementations, the microfluidic layer can be configured such that the spatial distribution of the microchannels is predetermined in accordance with the spatial distribution of the electrodes of the electrode layer, such that when the microfluidic layer and the electrode layer are placed in contact, a predetermined organized architecture of the microchannels relative to the electrodes can be achieved.

In some implementations, in order to align the electrodes 122 of each of the electrode grids 126 with the microchannels 65, one or more alignment features can be included in either one of the microfluidic layer 20 or the electrode layer 120, or both.

For instance, the electrode layer 120 can include a protruding member extending upwardly therefrom, and the microfluidic layer 20 can include a corresponding opening for engaging with the protruding member. The insertion of the protruding member of the electrode layer into the opening of the microfluidic layer can contribute to aligning the electrodes 122 of each of the electrode grids 126 with the microchannels 65 by limiting lateral and longitudinal movement of the microfluidic layer 20 relative to the electrode layer 120. It is to be understood that the reverse configuration can also be implemented, i.e., the microfluidic layer can include a protruding member extending downwardly toward the electrode layer, and the electrode layer can include a corresponding cavity for receiving the protruding member of the electrode layer therein.

In other implementations, the alignment feature can include a notch defined in the electrode layer 120, and the microfluidic layer 20 can include a protruding member configured to engage with the notch to limit lateral and longitudinal movement of the microfluidic layer 20 relative to the electrode layer 120. Alternatively, the notch can be defined in the microfluidic layer 20 and the electrode layer can include a protruding member that can be engaged with the notch of the microfluidic layer 20.

In yet other implementations, the alignment feature can include one or more features such as ridges, crests, furrows, grooves, and the like. For instance, the upper surface of the electrode layer 120 can include a series of ridges, and the lower surface of the microfluidic layer 20 can include a complimentary series of furrows, or vice versa, such that when the electrode layer 120 and the microfluidic layer 20 are coupled together, or placed in contact with each other, the electrode layer 120 and the microfluidic layer 20 can interlock and/or self-align to increase the stability of their positioning and facilitate the alignment of the electrodes 122 of the electrode layer 120 with the microchannels 65 of the microfluidic layer 20.

In some implementations, there can be an alignment feature provided in each corner region of the electrode layer 120, i.e., there can be four alignment features. In other implementations, there can be an alignment feature in two opposed corner regions, for instance one in the upper left-hand side of the electrode layer 120, and one in the lower right-hand side of the electrode layer 120. Any number of alignment features, from one and up, can be present to facilitate the alignment of the electrodes 122 of the electrode layer 120 with the microchannels 65.

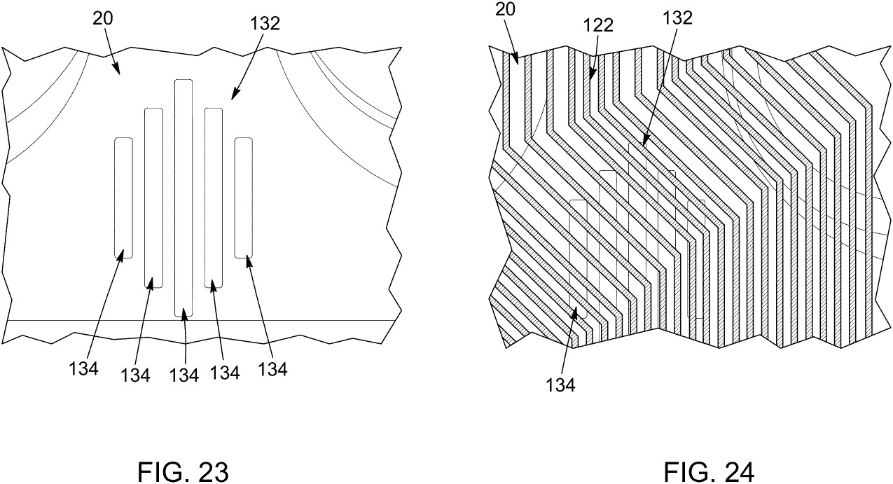

With reference to FIGS. 23 and 24, in yet other implementations, the alignment feature can be an alignment marking 132 provided on the microfluidic layer 20 to facilitate visual alignment of the electrodes 122 of the electrode layer 120 with the microchannels 65 of the microfluidic layer 20, or alignment of the microchannels 65 of the microfluidic layer 20 with the electrodes 122 of the electrode layer 120. An alignment marking can be any given pattern defined in the thickness of the microfluidic layer 20, or integrated in the microfluidic layer 20. Alternatively, the alignment marking can be added on the upper or lower surface of the microfluidic layer. The addition of the alignment marking on the upper surface or lower surface of the microfluidic layer can be done for instance via a sticker that is placed on the upper surface or lower surface of the microfluidic layer, the sticker including an alignment marking that can enable alignment of the microchannels with the electrodes. The alignment marking has a predetermined configuration that can be obtained based on the distribution of the electrodes, and that can be used to reproducibly position the microfluidic layer at a given position relative to the electrodes, or reproducibly position the electrodes at a given position relative to the microchannels. The alignment marking can thus be a visual reference that can be used to visually orient and position the microfluidic layer relative to the electrode layer, or vice versa. FIGS. 23 and 24 show an example of an alignment marking 132 that is integrated into the microfluidic layer 20. The alignment marking 132 shown in FIG. 23 includes a plurality of elongated markings 134. The pattern of the elongated markings 134 shown in FIG. 23 is one example among others, and it is to be understood that any combination of markings that can enable obtaining a pattern that can subsequently enable to serve as a point of reference for aligning the electrode can be suitable.

In some implementations, the alignment marking 132 can be determined following a test alignment of the microchannels with the electrodes. A test alignment of the microchannels with the electrodes can include aligning a given configuration of microchannels with a given configuration of electrodes by an alternate method, and once it is determined that the microchannels are aligned with the electrodes, an alignment marking can be added to the microfluidic layer such that subsequent matching of a microfluidic layer having the given configuration of microchannels with the given configuration of electrodes can be achieved based on the alignment marking.

In some implementations, when the microfluidic layer is used for culturing neuronal cells, the alignment marking 132 can enable to positioning the microfluidic layer such that one or more given electrodes are placed in electrical communication with the cells bodies, and one or more given electrodes are placed in electrical communication with the axons. For instance, in some implementations, the alignment marking 132 can include elongated markings 134 that are configured such that the top rows of electrodes is in electrical communication with the cell bodies of a first neuronal population, the middle rows of electrodes are in electrical communication with the axons, and the bottom rows of electrodes are in electrical communication with the cell bodies of a second neuronal population. This type of cooperation of the microfluidic layer with the electrode layer can enable to reproducibly placing given electrodes at selected locations of the microfluidic layer to enable electrical communication between the cells and the electrodes.

In the implementation shown in FIG. 24, the alignment marking 132, and more precisely the plurality elongated markings 134, is shown as being placed at a given position relative to the electrodes 122 of an electrode grid. This matching of the alignment marking 132 with the electrodes 122, which can be seen for instance by the alignment of the top of the right-hand side of the elongated markings 134 with the angled pathway of the electrodes 122, can be relied upon for aligning the microchannels of the microfluidic layer with the electrodes for that given configuration of microchannels and given configuration of electrodes.

In yet other implementations, the microfluidic layer 20 and the electrode layer 120 can be engaged with one another with a snap-on mechanism, or snap-fit mechanism, that results in a predetermined positioning of the microfluidic layer 20 relative to the electrode layer 120. The snap-on mechanism can reduce the need to use additional techniques or methods to bond the microfluidic layer 20 to the electrode layer 120, which can contribute to accelerating the overall experimental process. The snap-on mechanism can enable a non-permanent, or reversible, method of assembly of the microfluidic layer 20 and the electrode layer 120, and can also allow for the flexibility of using different coating materials. The predetermined positioning of the microfluidic layer 20 relative to the electrode layer 120 can enable aligning the electrodes 122 of each of the electrode grids 126 of the electrode layer 120 with the microchannels 65 of the microfluidic units 28, at least in part by limiting lateral and longitudinal movements of the microfluidic layer 20 relative to the electrode layer 120. The snap-on mechanism can be provided to ensure that the predetermined positioning of the microfluidic layer 20 relative to the electrode layer 120 remains constant once the microfluidic layer 20 is put in contact with the electrode layer 120. For instance, in some implementations and referring back to FIG. 17, the base layer 30 can include a plurality of hooks 34 that are adapted to slide and snap fit into corresponding slots in frame 14 of the base layer 30, the hooks 34 being adapted for detachably connecting the base layer 30 to the multi-well grid layer 10 and stabilizing the electrode layer 120 in position.

Although various examples of alignment features have been described above, it is to be understood that any alignment feature that can enable interlocking or stabilizing the microfluidic layer 20 with respect to the electrode layer 120 in a given position that results in the alignment of the electrodes 122 of the electrode grids 126 with the microchannels 65 of the microfluidic layer can be suitable.

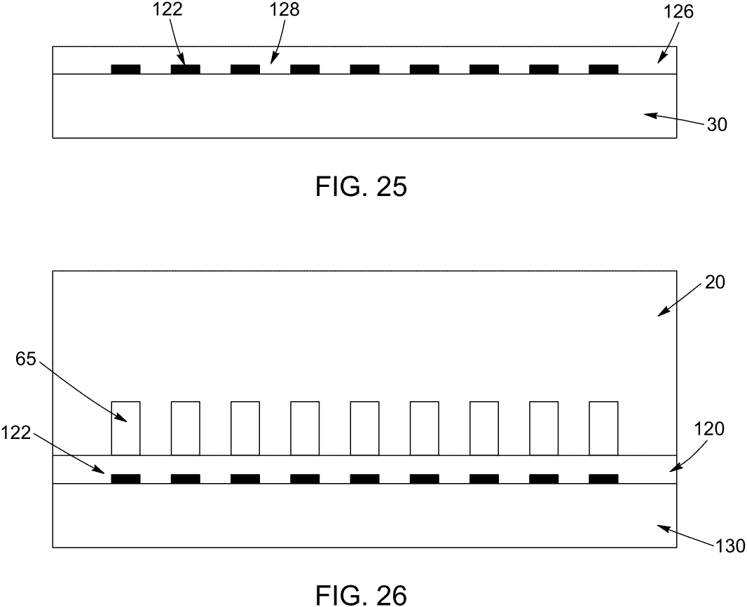

FIG. 25 illustrates a cross-sectional view of one of the electrode grids 126 of the electrode layer 120 shown in FIGS. 19 and 27. In this implementation of the electrode grid 126, the electrode grid 126 includes a protective coating 128 deposited onto the electrodes 122. The protective coating 128 can be made for instance of a polymer, such as Su-8, or any other polymer as known in the art. The electrode layer 120 is shown as being deposited onto the upper surface of the base layer 30.

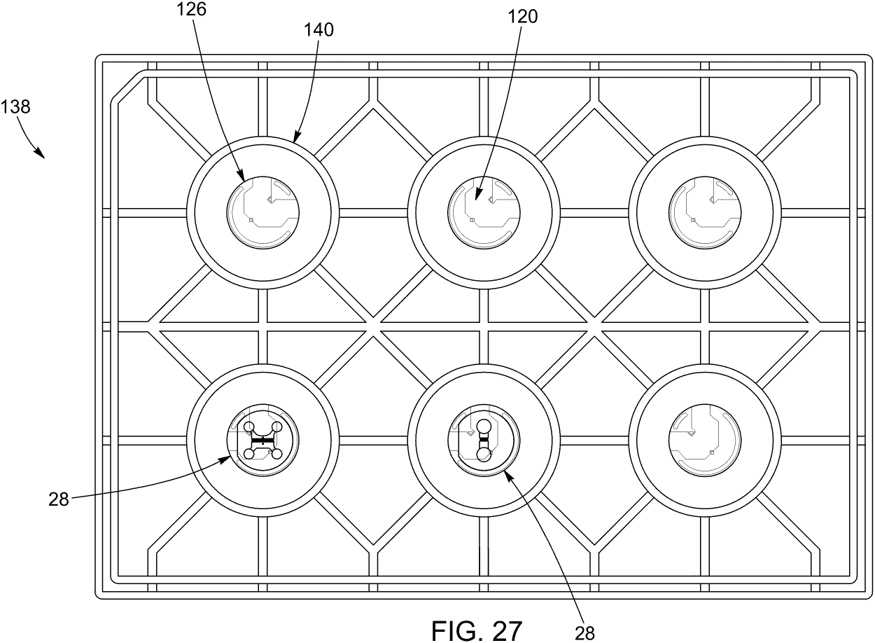

In some implementations, the electrode layer 120 can form part, or be integral with the base layer 30, such that no additional step of combining the electrode layer with the base layer has to be performed. In that respect, the electrode layer 120 shown in FIG. 18 can also be integrated into the base layer 30 of the multi-well device to form a unitary structure, such that the electrode layer 120 does not have to be manually superposed to the base layer 30. FIG. 26 illustrates the superposition of the microfluidic layer 20 onto the electrode layer 120, with the microchannels 65 being aligned with the electrodes 122. In this example, each of the microchannels is shown as intersecting with an electrode 122. In other implementations, there can be any other number of electrodes per microchannel, and there can be some microchannels that are adjacent to an electrode without the electrode directly intersecting the microchannels. The alignment of the electrodes with the microchannels, or of the microchannels with the electrodes, can be performed to achieve a given organization of the microchannels relative to the electrodes, and can take various forms depending on the intended application of the microfluidic layer.

In some implementations, the electrode layer 120 can be transparent. In the present description, the term “transparent” refers to the capability of an object of allowing electromagnetic radiation in a certain spectral region to pass therethrough without appreciable scattering. The term “translucent” refers to the capability of an object of allowing electromagnetic radiation in a certain spectral region to pass therethrough with appreciable scattering. The term “translucent” is generally synonymous with the term “partly transparent”. In this regard, it is understood that the term “transparent” includes not only “completely transparent”, but also “substantially transparent”, “sufficiently transparent”, and “partly transparent”. As such, unless specified otherwise, the term “transparent”, when used alone, is meant to encompass the term “translucent”. Providing an electrode layer that is transparent can contribute to facilitate the alignment of the electrodes with the microchannels or the alignment of the microchannels with the electrodes.

In some implementations, a microfluidic unit 28 as described herein can be used as a single microfluidic unit 28, instead of being provided as part of a microfluidic layer 20 that includes a plurality of microfluidic units 28, as illustrated for instance in FIGS. 2A, 2B and 18. When a single microfluidic unit 28 is used, the single microfluidic unit 28 can be inserted into a well of a multi-well plate. In such implementations, an electrode grid can be integrated into the bottom wall of the well, or an electrode grid can be deposited onto the bottom wall of the well.

FIG. 27 illustrates an example of a multi-well plate 138 that includes six wells 140, each of the wells 140 including an electrode layer 120 that is integrated into the bottom wall of the well 140, and which can also be referred to as the base layer. Each electrode layer 120 includes an electrode grid 126. In FIG. 27, a single microfluidic unit 28 is shown in a respective well 140 of the six wells, each of the single microfluidic units 28 having a respective configuration, i.e., one of the single microfluidic units includes two inlets and two outlets as described herein, and the other one of the microfluidic units includes one inlet and one outlet. The multi-well plate 138 can be used in cooperation with a multi-well plate reader equipment configured for reading signals from the electrodes 122.

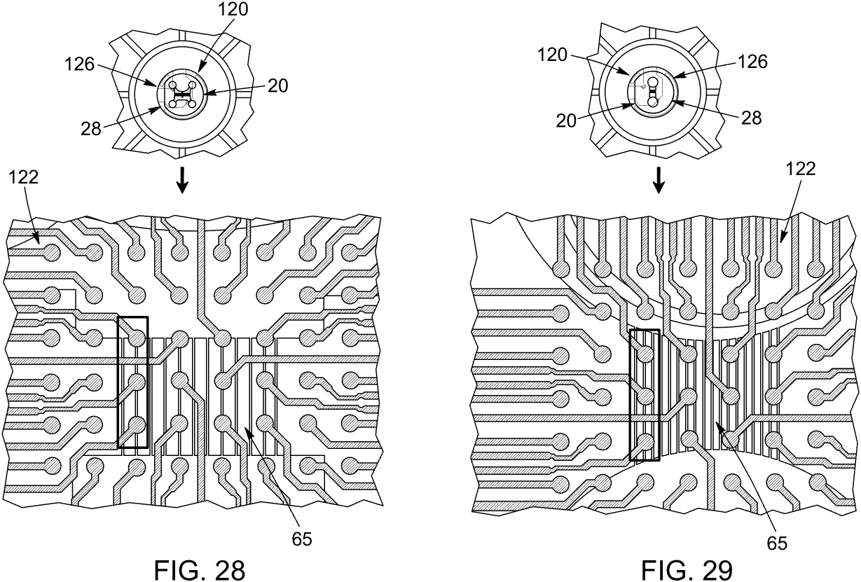

FIGS. 28 and 29 illustrate, for each of the single microfluidic units 28 shown in FIG. 27, that a spatial organization of the microchannels of the microfluidic unit can be obtained with respect to the electrodes 122 of the electrode grid 126, resulting in the microchannels 65 being aligned with the electrodes 122 of the electrode grid 126. For instance, the microchannels can be aligned with the tips 124 of the electrodes 122 that serve as measuring points. In the implementation shown in FIG. 28, three electrode tips are shown as intersecting a respective microchannel. In the implementation shown in FIG. 29, three electrode tips are shown as being intersected across an entire diameter thereof by two substantially parallelly extending microchannels 65. It is to be understood that in some implementations, the number of microchannels located adjacent or intersecting an electrode can be a secondary consideration, and that the predetermined organized architecture of the microchannels relative to the electrode once the microchannels and the electrodes are aligned can be a desired objective to promote better contact with the cells, such as neuronal cells, present in the microchannels with the electrodes.

When a single microfluidic unit 28 is to be inserted into a well 140 having an electrode layer 120 deposited onto, or integrated into, a bottom wall of the well 140, either one of the single microfluidic unit 28 or the electrode grid 126, or both, can include an alignment feature to enable the alignment of the microchannels with the electrodes.

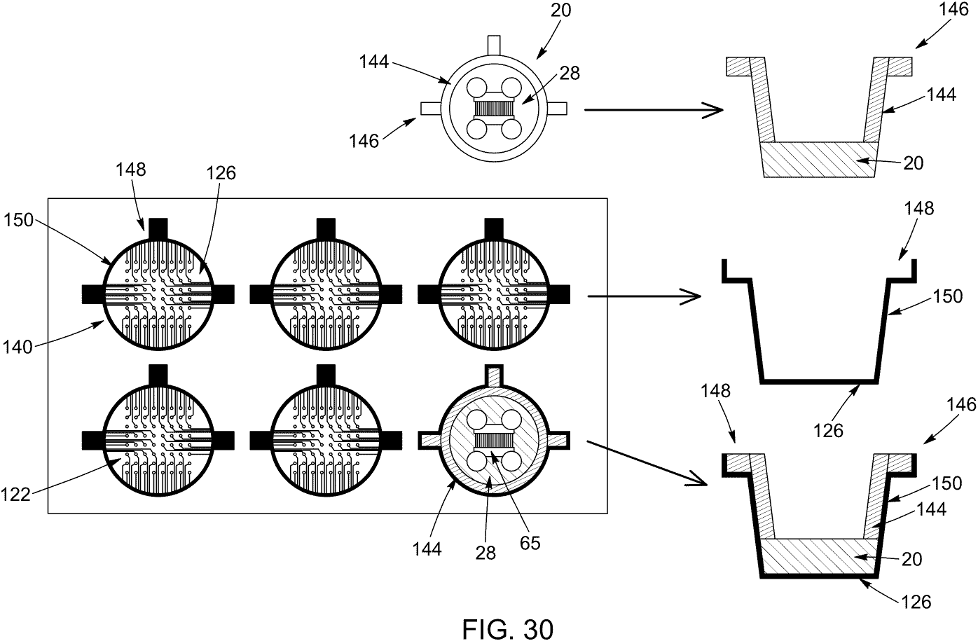

In some implementations, and with reference to FIG. 30, the single microfluidic unit 28 can include an alignment frame 144 coupled to the microfluidic unit 28, the microfluidic unit 28 and the alignment frame 144 being fixedly engaged to prevent rotation of the microfluidic unit 28 within the alignment frame 144. The alignment frame 144 can include one or more alignment tabs 146 extending outwardly from the alignment frame 144. In the implementation shown, the alignment frame 144 includes three alignment tabs 146. It is to be understood that any number of alignment tabs 146 can be present, from one and up. In other words, at least one alignment tab 146 can extend outwardly from the alignment frame 144. In turn, the well 140 includes a corresponding number of alignment tab-receiving cavity 148 defined in the peripheral wall 150 of the well, the alignment tab-receiving cavity 148 being configured for receiving an alignment tab 146 therein. The alignment tab-receiving cavity 148 can be located at a predetermined location around the periphery of the peripheral wall 150 such that when the alignment tab 146 of the alignment frame 144 is inserted into the alignment tab-receiving cavity 148, the alignment frame 144 interlocks with the peripheral wall 150 of the well. The alignment tab-receiving cavity 148 can thus serve as a guide to orient the microfluidic unit 28 for insertion into the well 140 and onto the electrode layer 120, by imposing a predetermined interaction between the alignment tab-receiving cavity 148 and the alignment tab 146. It is to be understood that the reverse configuration is also possible, with the peripheral wall of the well 140 having at least one alignment tab, and the alignment frame 144 having a corresponding alignment tab-receiving cavity.

Still referring to the implementation shown in FIG. 30, the alignment frame 144 includes three alignment tabs 146, with three alignment tab-receiving cavities 148 being defined in the peripheral wall of the well at predetermined locations. The alignment tabs 144 are provided at 0°, 90° and 270°of the circular well 140, with the alignment tab-receiving cavities 148 being provided at corresponding locations. Given that no alignment tab-receiving cavity 148 is provided at 180°, the alignment frame 144 and associated alignment tabs 146 can be inserted into the well 140 according to a single orientation of the microfluidic unit 28. If the alignment frame 144 and associated alignment tabs 146 were rotated either clockwise or counterclockwise for any number of degrees up to 90°, the alignment tabs 146 would not fit within corresponding alignment tab-receiving cavities 148, and if the alignment frame 144 and associated alignment tabs 146 were rotated clockwise or counterclockwise for 90°, there would be no alignment tab-receiving cavity 148 at 180° for receiving a corresponding alignment tab 146. Other configurations of the alignment tabs 146 and associated alignment tab-receiving cavities 148 are also possible, and the example shown in FIG. 30 is for illustrative purposes only. The interaction between the alignment tabs 146 and associated alignment tab-receiving cavities 148 can enable the microfluidic unit 28 to be deposited onto the electrode layer 120 according to a substantially rotationally constant orientation, such that once deposited onto the electrode layer 120, the microchannels 65 are aligned with the electrodes of the electrode layer 120.

Although the alignment tab-receiving cavities 148 have been exemplified as being provided at 0°, 90° and 270°of the circular well and the alignment tabs 146 have also been exemplified at 0°, 90° and 270° of the circular microfluidic unit 28, any other combination of angles is also possible to achieve a single interlocking interaction between the microfluidic unit 28 and the electrode layer 120.

In other implementations, still when a single microfluidic unit 28 is to be inserted into a well having an electrode layer 120 deposited onto, or integrated into, a bottom wall of the well, the alignment feature for enabling alignment of the microchannels with the electrodes can take the form of a protruding member extending upwardly from the bottom wall of the well or from the electrode layer, and the microfluidic layer can include a corresponding opening for engaging with the protruding member. The insertion of the protruding member of the bottom wall of the well or of the electrode layer into the opening of the microfluidic layer can contribute to aligning the electrodes of the electrode layer with the microchannels of the microfluidic unit by limiting lateral, longitudinal and rotational movement of the microfluidic layer relative to the electrode layer.

It is to be understood that the reverse configuration can also be implemented, i.e., the bottom wall of the well or the electrode layer can include a cavity and the microfluidic layer can include a protruding member extending downwardly toward the electrode layer for being received into the cavity of the bottom wall or of the electrode layer. In implementations where the microfluidic unit 28 has a circular shape, such as shown in FIG. 30, the number of protruding members and associated cavities can be chosen to limit the rotational movement of the microfluidic unit and to ensure that the microfluidic unit remains at a substantially rotationally constant orientation upon deposition onto the electrode layer.

Similarly to the implementation shown in FIG. 30 with the alignment frame 144 and associated alignment tabs 146, there can be three protruding members extending upwardly from the bottom wall of the well or of the electrode layer, provided at 0°, 90° and 270°, and associated openings defined in the microfluidic layer to engage with protruding members for interlocking the microfluidic layer at a substantially rotationally constant orientation relative to the electrode layer, such that once deposited onto the electrode layer, the microchannels of the microfluidic unit are aligned with the electrodes of the electrode layer.

In implementations where the bottom wall of the well or the electrode grid includes a cavity and the microfluidic unit includes a protruding member extending downwardly toward the electrode layer for being received into the cavity of the bottom wall or of the electrode layer, there can be three protruding members extending downwardly from the microfluidic unit, provided at 0°, 90° and 270°, and associated cavities defined in the bottom wall of the well or in the electrode layer to engage with protruding members for interlocking the microfluidic unit at a substantially rotationally constant orientation relative to the electrode layer, such that once deposited onto the electrode layer, the microchannels of the microfluidic unit are aligned with the electrodes of the electrode layer.

In yet other implementations, still when a single microfluidic unit is to be inserted into a well having an electrode layer deposited onto, or integrated into, a bottom wall of the well, the alignment feature can include one or more features such as ridges, crests, furrows, grooves, and the like. For instance, the upper surface of the bottom wall of the well or of the electrode layer can include a series of ridges, and the lower surface of the microfluidic layer, i.e., underneath the microfluidic unit, can include a complimentary series of furrows at specific locations such that when the microfluidic unit is deposited onto the electrode layer so that the microfluidic unit and the electrode layer can interlock, the microfluidic unit is deposited at a substantially rotationally constant orientation on the electrode layer to facilitate alignment of the electrodes with the microchannels of the microfluidic unit.

In yet other implementations, still when a single microfluidic unit is to be inserted into a well having an electrode layer deposited onto, or integrated into, a bottom wall of the well, the alignment feature can be an alignment marking as described above. In such implementations, an alignment marking can be provided on the microfluidic layer to facilitate alignment of the electrodes with the microchannels or alignment of the microchannels with the electrodes. An alignment marking can be a given pattern defined in the thickness of the microfluidic layer, or integrated in the microfluidic layer. Alternatively, the alignment marking can be added on the upper or lower surface of the microfluidic layer, for instance as a sticker.

The electrodes 122 can be configured to provide an electrical signal to stimulate the neuronal cells growing in the microchannels, or any other type of cells growing in the microchannels. The electrodes 122 can also be configured to collect, and/or record, and/or measure, and/or detect the response of cells to stimulation. In some implementations, the same electrode can be configurable to sequentially perform different actions. For instance, the electrode can be configured to collect a signal at a given timepoint, and at a subsequent timepoint, the electrode can be configured to provide an electrical signal. In some implementations, the electrode can be configured to detect an optical signal or an electrical signal.

The electrodes can enable providing electrical read-outs comprising one or more of potential recordings, impedance spectroscopy, voltammetry and amperometry.

In some implementations, the electrodes can comprise at least one metallic electrode, at least one metal oxide electrode, at least one carbon electrode, a multi electrode array, and/or at least one field effect transistor detectors. The substrate containing the electrodes can be fabricated from a stiff material or a flexible material to facilitate obtaining a tight fit and/or a leak-proof fit, with the microfluidic layer.

In some implementations, any other type of sensors can be used to stimulate cells that are present in the microchannels or to measure responses of cells that are present in the microchannels to stimulation. Examples of sensors can include optical sensors, chemical sensors, and electrical sensors, for instance. In some implementations, the sensors can be made of graphene, and can be used to detect and measure physical, biological and chemical signals such as electrical activity and/or the concentration of specific compounds.

In some implementations, an electronic device can be provided in ohmic connection with the electrodes described above. The electronic device can include for instance a sensing device or a stimulating device, and can be configured for providing electrical read-outs comprising one or more of potential recordings, impedance spectroscopy, voltammetry and amperometry. The electronic device can be located within the reservoir of a cell culture plate or an insert well of a multi-well insert, or be provided in proximity thereof.

In some implementations, a sensor configured for stimulating neuronal cells present in the microchannels, measuring spontaneous activity of cells as well as a response from the neuronal cells present in the microchannels to stimulation, and/or providing an output and/or receiving an input can be provided. The sensor can include for instance an optical or an electrical transducer.

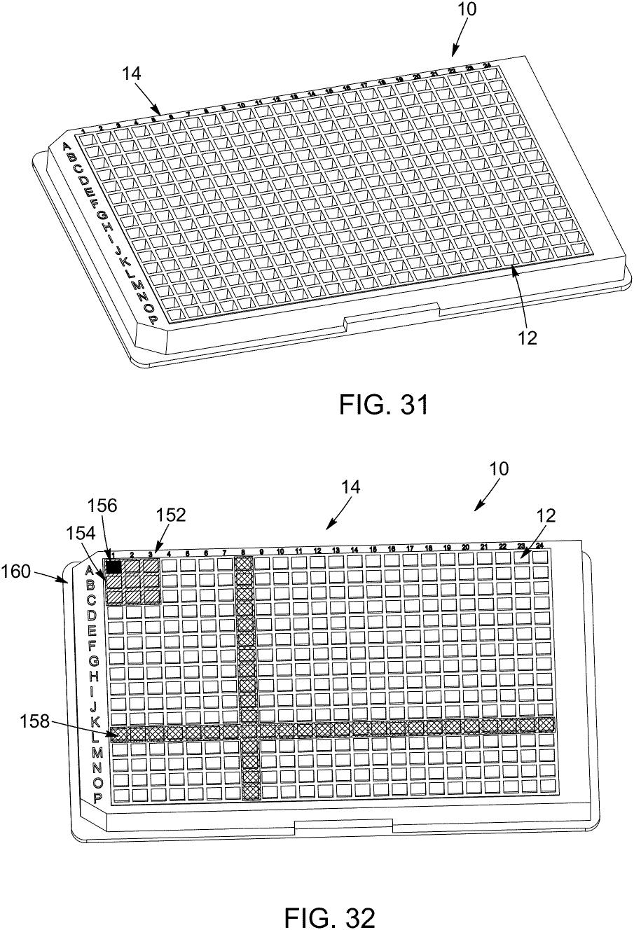

Referring now to FIGS. 31 to 33, an implementation of a multi-well grid layer is shown. In accordance with the concepts described above, the multi-well grid layer 10 includes a rectangular frame 14 surrounding a central portion that includes a plurality of wells 12 that are bottomless. In the implementation shown in FIGS. 31 to 33, the multi-well grid layer 10 includes 384 bottomless wells, but it is to be understood that the multi-well grid layer 10 can also include any number of wells, such as 6, 12, 24, 48, 96, or 1536 wells.

Referring more particularly to FIG. 32, the multi-well grid layer 10 includes a well identification feature 160 provided as well as markings 152. Each well marking 152 is configured to at least partially surround specific wells 12 of the multi-well grid layer 10. In the implementation shown in FIG. 32, a total of twenty-four well markings 152 are shown, each one of the twenty-four well markings 152 being associated with a corresponding microfluidic unit 28 of the microfluidic layer 20. In the implementation shown in FIG. 32, the multi-well grid layer 10 being made of a dark-coloured material, the well markings 152 are provided in lighter color to enhance the contrast between the multi-well grid layer 10 and the well markings 152. It is to be understood that any color of the well markings that visually contrasts sufficiently with the multi-well grid layer to identify specific wells associated with at least a portion of the microfluidic unit can be suitable.

In the implementation shown, the multi-well grid layer 10 is configured for use with a microfluidic layer as shown in FIGS. 2A and 2B, and with the microfluidic unit 28 having a configuration such as shown in FIG. 3B. Accordingly, in FIG. 32, each well marking 152 is configured to at least partially surround an overall microfluidic unit covering a total of nine wells, and each of the four wells that is in fluid communication with a corresponding one of the inlets and outlets 67a, 67b, 67c, 67d. The portion of the well marking 152 surrounding the overall microfluidic unit can be referred to as an outer well marking 154 (schematically shown by the hatched square in FIG. 32), and the portion of the well marking 152 surrounding each of the four wells that is in fluid communication with a corresponding one of the inlets and outlets 67a, 67b, 67c, 67d can be referred to as an individual well marking 156 (schematically shown by the dark square in FIG. 32). It is to be understood that in some implementations, the well marking 152 can include either one of the outer well marking or the individual well marking, or both. The well marking 152 of the overall microfluidic unit and/or of the inlets and outlets 67a, 67b, 67c, 67d can facilitate visual identification of the wells into which liquids, particles, compounds and/or cells can be fed to be added in the microfluidic unit, and the visual identification of the wells from which liquids, particles, compounds and/or cells can be taken from to be removed from the microfluidic unit. For instance, the well making 152, and more particularly, the individual well markings 156, of FIG. 32 identifies wells A1, A3, C1 and C3 that would be aligned with the inlets and outlets 67a, 67b, 67c, 67d of the microfluidic unit 28, with the outer well marking 154 surrounding wells A1, A2, A3, B1, C1, C2, C3 and B3. In this implementation, an array of wells 158 (schematically shown by the hatched rectangle) between successive microfluidic units is left without individual well marking, as these wells are not in fluid communication with inlets and outlets of the microfluidic layer and are thus not typically configured for receiving a fluid therein when the microfluidic units of the microfluidic layer 20 is configured as shown in FIGS. 2B and 3B.

The outer well marking 154, the individual well marking 156 and the array of wells 158 are shown for illustrative purposes only, and it is to be understood that the well marking 152 can be adapted according to the number of wells of the multi-well grid layer 10 and the microfluidic layer 20 and corresponding microfluidic units 28 that are used, to achieve the visual identification of the wells corresponding to the inlet(s) and outlet(s) of the microfluidic unit.

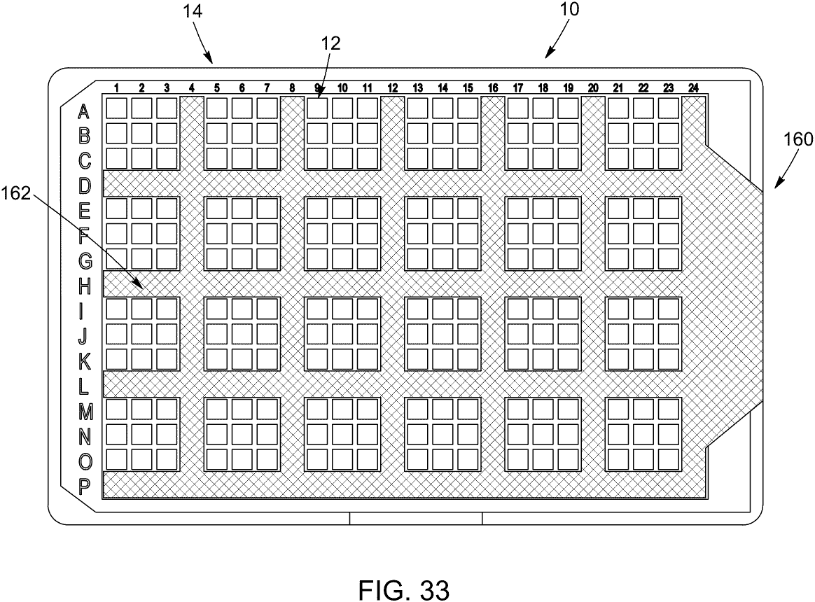

FIG. 33 illustrates another implementation of a multi-well grid layer 10 that is used in cooperation with a well identification feature 160. In FIG. 33, the well identification feature 160 is provided as a well identification layer 162 that is superposable to the upper surface of the multi-well grid layer 10 to enable visual identification of at least a portion of the microfluidic unit, such as the wells corresponding to the inlet(s) and outlet(s) of the microfluidic unit.

In the implementation shown in FIG. 33, the well identification layer 162 is made of a “mask” that is placed on the upper surface of the multi-well grid layer 10, to block the arrays of wells between successive and spaced-apart microfluidic units, as these wells are not in fluid communication with inlets and outlets of the microfluidic layer and are thus not typically configured for receiving a fluid therein when the microfluidic units 28 of the microfluidic layer 20 is configured as shown in FIGS. 2B and 3B. In other words, the well identification layer 162 can include columns provided in between longitudinally spaced-apart microfluidic units of the plurality of microfluidic units, and/or rows provided in between laterally spaced-apart microfluidic units of the plurality of microfluidic units.

In some implementations, the well identification layer 162 shown in FIG. 33 can be used in addition to the well markings 152 of FIG. 32, for instance to cover the arrays of wells 158 (schematically shown by the purple rectangle in FIG. 32) between successive and spaced-apart microfluidic units.

The well identification layer 162 can have any color to facilitate identification of the wells associated with a corresponding microfluidic unit. In the implementation shown in FIG. 33, the well identification layer 162 is illustrated in a light color, to enhance the visual contrast with the dark-colored multi-well grid layer 10.

In some implementations, the well identification layer 162 can be removable from the upper surface of the multi-well grid layer 10 to be reused multiple times with distinct multi-well grid layers 10. The well identification layer 162 can be a sticker-type well identification layer 162 that can be temporarily affixed to the upper surface of the multi-well grid layer 10, to avoid the well identification layer 162 to be displaced, e.g., slid over the upper surface of the multi-well grid layers 10, during use. In some implementations, the well identification layer 162 can have a configuration that is different than the configuration shown in FIG. 33. For instance, the well identification layer 162 can include a plurality of well identification layer 162, one for each of the microfluidic units of the microfluidic layer. The well identification layer 162 can surround the outer periphery of wells, or partially surround the outer periphery of wells, such as shown in FIG. 33.

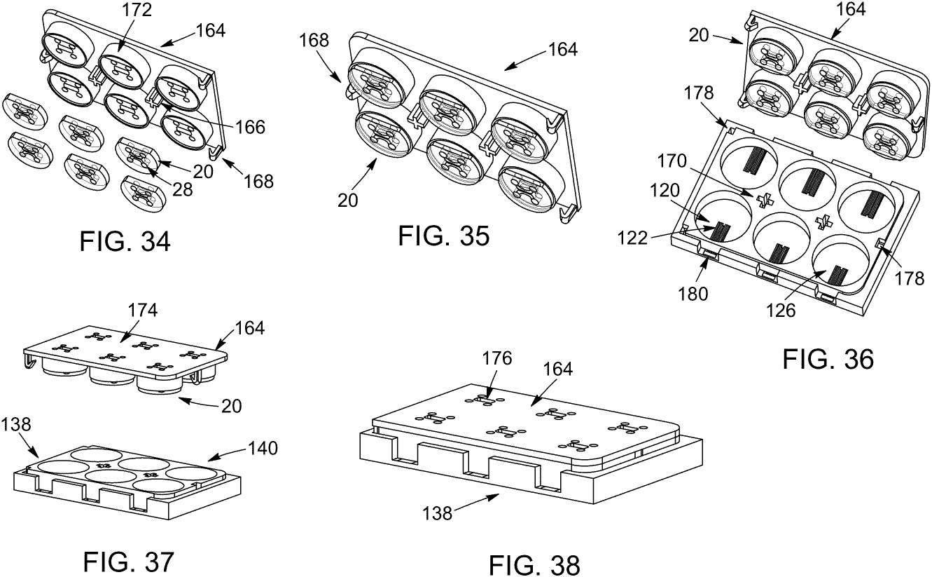

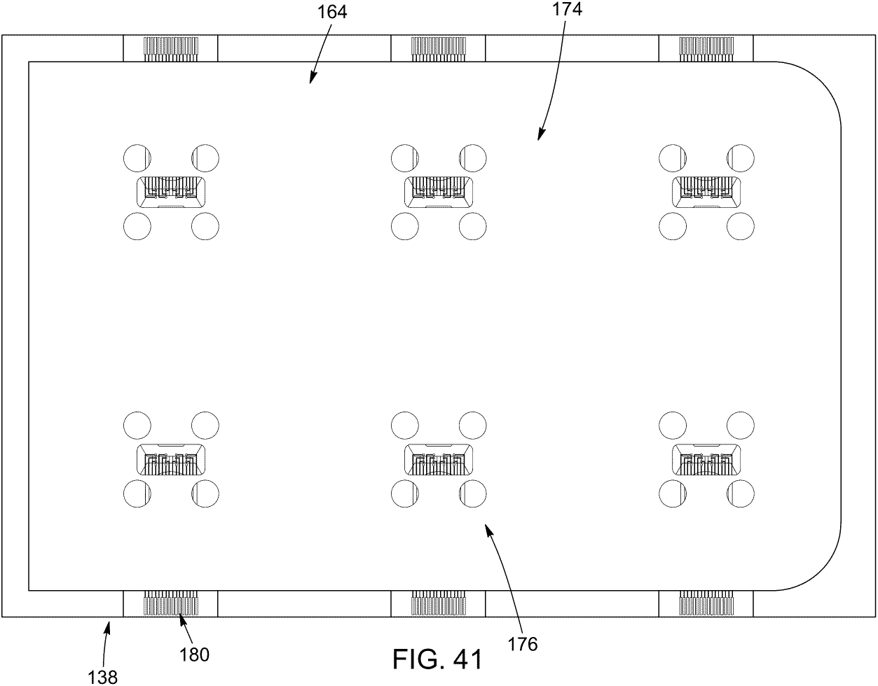

Referring now to FIGS. 34-43, another implementation of a device for culturing cells and that enables alignment of the microchannels of a microfluidic unit with electrodes of an electrode layer to achieve to achieve a predetermined organized architecture of the microchannels of the microfluidic layer relative to the electrodes is shown.

In this implementation, the cell culture device includes a microfluidic layer engagement frame 164 that is engageable with a plurality of microfluidic layers 20 each having a microfluidic unit 28, the plurality of microfluidic layers 20 being provided in an adjacent and spaced-apart relationship. In the implementation shown, the microfluidic layer engagement frame 164 includes a plurality of downwardly extending tubular portions 172 extending downwardly from a base wall 174 of the microfluidic layer engagement frame 164, the number of downwardly extending tubular portions 172 corresponding to the number of microfluidic layers 20 with which the microfluidic layer engagement frame 164 is to be engaged. The size and configuration of the downwardly extending tubular portions 172 can be determined according to the size and configuration of the wells 140 of the multi-well plate 138. For instance, in the implementation shown, the multi-well plate 138 includes six wells 140, and the microfluidic layer engagement frame 164 includes a corresponding six downwardly extending tubular portions 172. The wells 140 of the multi-well plate 138 each include an electrode layer 120 that can be integrated into the bottom wall of the well 140 or be deposited onto the bottom wall of the well 140. Each electrode layer 120 includes electrodes forming an electrode grid 126. In the implementation shown in FIG. 40, the electrode grid 126 is a 4 × 4 circular grid, but it is to be understood that other configurations of the electrode grid 126 are of course possible depending on the microfluidic layer used in combination therewith and/or the intended application.

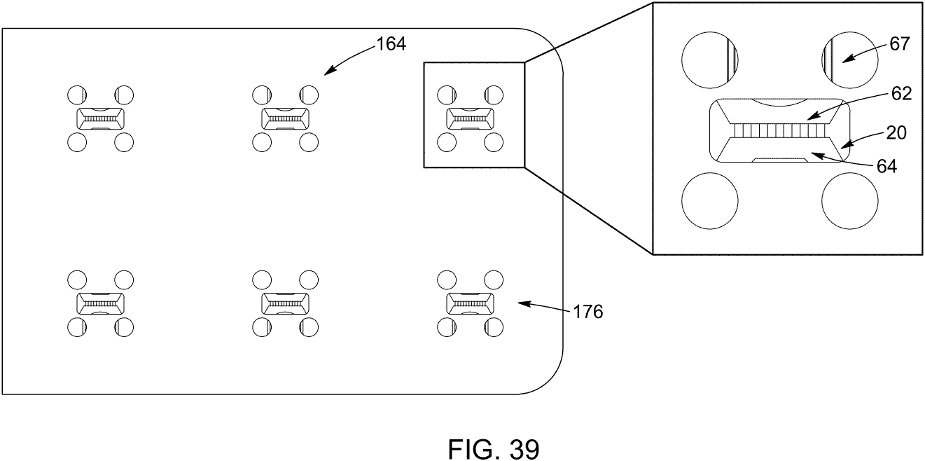

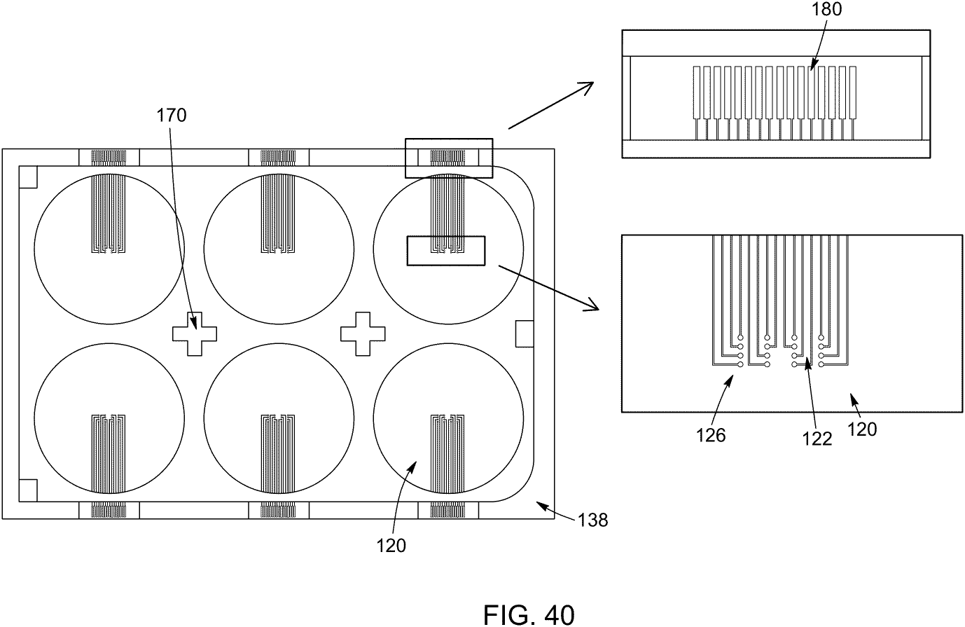

In the implementation shown, the base wall 174 includes microfluidic layer openings 176 defined therein to enable fluid communication with compartments and microchannels of a corresponding microfluidic unit 28. For instance, in FIGS. 34-43, the microfluidic layer 120 includes a microfluidic unit 28 that has a configuration similar to the configuration of the microfluidic unit shown in FIG. 5 or FIG. 30, and includes a total of four inlets and outlets 67a, 67b, 67c, 67d and microchannels 65 extending between a first compartment 62 and a second compartment 64. Accordingly, the base wall 174 of the microfluidic layer engagement frame 164 includes corresponding microfluidic layer openings 176 exposing key components of each of the microfluidic units 28, such as shown in FIG. 39. Although a total of five microfluidic layer openings 176 are shown for each microfluidic unit 28, it is to be understood that other configurations are also possible, such as a single microfluidic layer opening, or three microfluidic openings defined in the base wall 174 of the microfluidic layer engagement frame 164, or any other configuration that enable access to the compartments of the microfluidic unit 28. In some implementations, the presence of the microfluidic openings 176 can facilitate visual identification of the components of the microfluidic unit 28 into which liquids, particles, compounds and/or cells can be added, and the visual identification of the components of the microfluidic unit 28 from which liquids, particles, compounds and/or cells can be taken from. In some implementations, the presence of the microfluidic openings 176 can also guide a user’s pipette within a constraint area to facilitate the addition or retrieval of liquids, particles, compounds and/or cells from the microfluidic unit.

In some implementations, the microfluidic layer engagement frame 164 can include a multi-well plate alignment feature 166 extending downwardly from the base wall 174 toward the multi-well plate 138, the multi-well plate alignment feature 166 being insertable into an alignment feature receiving opening 170 defined in the multi-well plate 138 to align the microfluidic layer engaging frame 164 with the multi-well plate 138. The reverse configuration is also possible, with an alignment feature extending upwardly from the multi-well plate and that is insertable into a corresponding receiving opening defined in the microfluidic layer engagement frame 164.

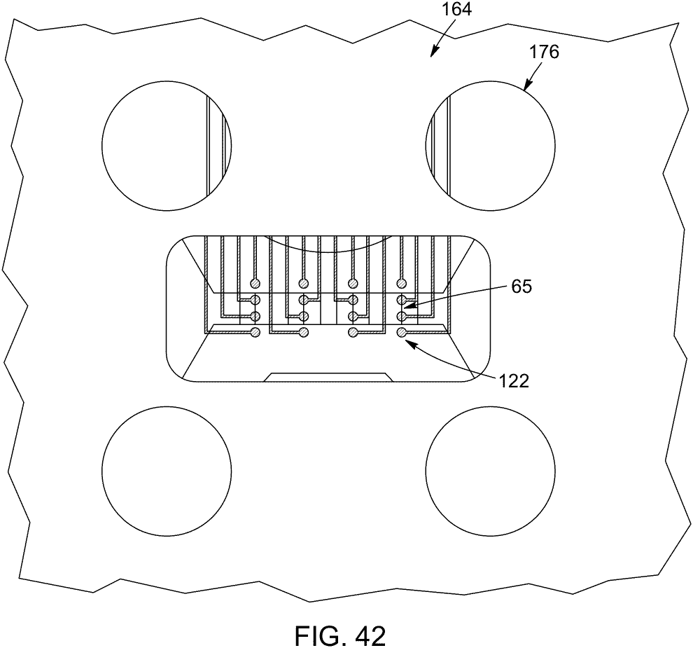

The multi-well plate alignment feature 166 can take various shapes and be of various sizes, as long as the multi-well plate alignment feature 166 enables coupling with the alignment feature receiving opening 170 such that the microfluidic layer engagement frame 164 is at a predetermined position relative to the multi-well plate 138. The number and spatial distribution of the multi-well plate alignment feature(s) 166 and associated alignment feature receiving opening(s) 170 can also vary from the implementation shown in FIGS. 34-43. For instance, there can be four multi-well plate alignment features 166, one provided in each corner region of the base wall 174, and four associated alignment feature receiving openings 170 defined in the multi-well plate 138, or there can be a single microfluidic layer engagement frame 164 and an associated alignment feature receiving opening 170. These examples are provided for illustrative purposes only, and are not meant to be limitative in any way. In addition, the number and spatial distribution of the multi-well plate alignment feature(s) 166 and associated alignment feature receiving opening(s) 170 can vary and be adapted depending on the number of wells of the multi-well plate 138. In the implementation shown, the multi-well plate alignment feature 166 and the alignment feature receiving opening 170 are exemplified as being cross-shaped, but the multi-well plate alignment feature 166 and the alignment feature receiving opening 170 can have any shape that enables limiting the movement, e.g., lateral and longitudinal displacements, of the microfluidic layer engagement frame 164 relative to the multi-well plate 138. The cooperation of the multi-well plate alignment feature 166 with the alignment feature receiving opening 170 can thus contribute to ensure that the microfluidic layer engagement frame 164 is positioned at a predetermined location relative to the multi-well plate 138 which in turn, can also enable the plurality of microfluidic layers 20 and the microfluidic units 28 to be positioned at a given location relative to the electrodes 122 to achieve the predetermined organized architecture of the microchannels of the microfluidic layer relative to the electrodes. FIG. 42 illustrates the enlarged portion of FIG. 39 once the unitary structure comprising the microfluidic layer engaging frame 164 and the plurality of microfluidic layers 20 has been coupled with the multi-well plate 138, and illustrates the alignment of the electrodes 122 of an electrode layer 120 with the microchannels 65 of a microfluidic layer 20.

In some implementations, the microfluidic layer engaging frame 164 can include an engagement feature 168 engageable with the multi-well plate 138. The engagement feature 168 can take various forms, and can be configured so as to enable alignment of the microfluidic layer engaging frame 164 relative to the multi-well plate 138. In the implementation shown in FIGS. 34-43, three engagement features 168 are present, each one of the three engagement features 168 extending downwardly from the base wall 174 and being shaped as a hook, or as a lever, that is engageable with an engagement feature connector 178 defined in the multi-well plate 138. In some implementations, the engagement feature 168 is engageable with the engagement feature connector 178 via a snap-on mechanism, also referred to as a snap-fit mechanism. The snap-on mechanism can be for instance a cantilever snap-on mechanism, as in the illustrated implementation. Alternatively, the engagement feature 168 can be any type of mechanical fastener that enables engagement with the multi-plate layer 138, either permanently or reversibly.

In some implementations and as shown in FIGS. 34-43, the microfluidic layer engaging frame 164 both a multi-well plate alignment feature 166 and an engagement feature 168. Alternatively, the microfluidic layer engaging frame 164 can include either one of a multi-well plate alignment feature 166 or an engagement feature 168.

The engagement features 168 are distributed at strategic locations around the periphery of the base wall 174 to provide a stable engagement of the microfluidic layer engaging frame 164 with the multi-well plate 138, and thus of the microfluidic layers 20 and the electrodes layers 120. In some implementations, the engagement of the engagement features 168 with the engagement feature connector 178 can be such that a light pressure is applied to hold the microfluidic layer engaging frame 164 and the multi-well plate 138 together. In some implementations, the light pressure can result from the engagement of the engagement feature 168 with the engagement feature connector 178 via the snap-on mechanism described above.

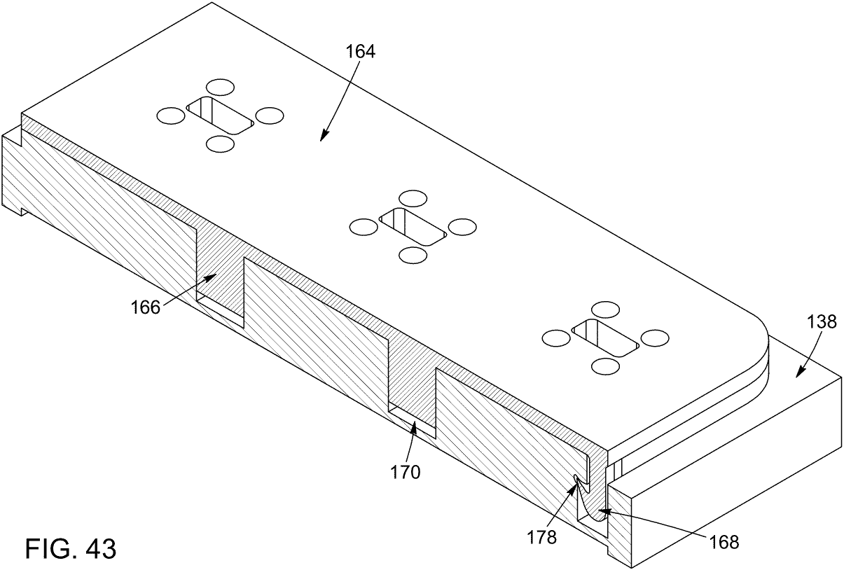

FIG. 43 shows a cross-sectional view of the combination of a microfluidic layer engaging frame 164 and a multi-well plate 138, taken along a longitudinal axis in a middle portion thereof. This cross-sectional view of the combination of a microfluidic layer engaging frame 164 and a multi-well plate 138 illustrates the interaction of the multi-well plate alignment feature 166 with the alignment feature receiving opening 170, and the interaction of the engagement feature 168 with the engagement feature connector 178. In this implementation, the cross-sectional view shows that the multi-well plate alignment feature 166 falls substantially flush with the wall of the alignment feature receiving opening 170, which can contribute to stabilize the microfluidic layer engaging frame 164 relative to the multi-well plate 138. When the expression “substantially flush” is used to describe the cooperation between the multi-well plate alignment feature 166 and the alignment feature receiving opening 170, it is meant that there can be a frictional contact between the multi-well plate alignment feature 166 and the alignment feature receiving opening 170, or there can be a gap ranging from about 0.1 µm to about 5 mm, for instance. Furthermore, the cross-sectional view of FIG. 43 illustrates the engagement of the engagement feature 168 with the engagement feature connector 178, when the engagement feature 168 is shaped as a cantilever snap-fit.

In some implementations, the base wall 174 can include an alignment feature that enables positioning a corresponding one of the plurality of microfluidic layers 20 according to a predetermined orientation. For instance, in the implementation shown in FIGS. 34-43, the surface of the base layer 174 that contacts a microfluidic layer 20 can include a protruding section (not shown) having a shape that is complementary to the shape of the microfluidic layer 20, such that when the microfluidic layer 20 is placed in contact with the surface of the base layer 174, the microfluidic layer 20 can be positioned at a rotationally constant orientation, i.e., in a predetermined orientation. In the implementation shown in FIGS. 34-43, the protruding member would thus be shaped as a croissant (half-moon) that would be complementary to the shape of the illustrated microfluidic layer 20. Once the microfluidic layer 20 is positioned at a predetermined location, the unitary structure comprising the microfluidic layer engaging frame 164, the combination of the microfluidic layers 20 with the multi-well plate 138 enables depositing the microfluidic layers onto a corresponding electrode layer 120 such that a predetermined organized architecture of the microchannels relative to the electrodes can be achieved.

Alternatively, in some implementations, the microfluidic layers 20 can be integral with the microfluidic layer engaging frame 164, which can also ensure that the combination of the microfluidic layers 20 with the multi-well plate 138 can result in the microfluidic layers being deposited onto a corresponding electrode layer 120 such that a predetermined organized architecture of the microchannels relative to the electrodes can be achieved.

In the implementation shown in FIGS. 34-43, and with reference more particularly to FIG. 40, the electrodes 122 of each of the electrode layers 120 of the multi-well plate 138 can be electrically connected by wires to a series of terminals 180. The terminals 180 are provided in proximity of the outer periphery of the multi-well plate 138, which can facilitate the interfacing of the terminals 180 with external equipment, such as a multi-well plate reader equipment configured for reading signals from the electrodes 122.

### EXAMPLES

### Example 1: Assay to Compare the Effects of Two Compounds on Axonal Growth and Neuronal Network Formation

Different compounds can affect the capacity of axons to grow and to form connections to create a functional network [Olivia Spead, Fabienne E Poulain Trans-Axonal Signaling in Neural Circuit Wiring. Int J Mol Sci 2020 Jul 21;21(14):5170.] In this example, we have used the device of the current technology to grow cortical neurons derived from rat in compartmentalized cultures to compare neuronal morphology and circuitry formation in the presence of two different compounds.

The upper surface (37) of the base layer (30) was coated with a solution of 100 µg/ml of PDL for 2 h at 37° C. and 5% CO2. Next, the upper surface was washed and assembled with (i) a microfluidic layer (20) comprising 24 microfluidic units (28) (each unit comprising a pair of inlets/outlets and one central chamber divided in two compartments by 120 microchannels and (ii) a multi-well grid layer (10) consisting of a 384-well plate (e.g., as illustrated in FIG. 7).

Animal procedures were performed in accordance with the Canadian Council on Animal Care Guidelines and were approved by the INRS Animal Care and Use Committee. Sprague Dawley rats were provided by Charles River Laboratories. The dissection of cortices of rat embryos at gestational date E19 was performed and cells were dissociated and suspended in a solution with 1 million cells/ml.

Next, 10 µl of cell suspension (20,000 cells) were added to the top well on the right side of each of the 24 microfluidic units in the 384-well plate. For example, cells were added to wells A3, A7, A11, A15, A19, A23 and to wells of the same number on rows E, I and M.

After incubation of the microplates for 20 min to promote cell adhesion to the upper surface (37) of the base layer (30), 50 µl of preheated cell medium was added to the top wells of each microfluidic unit and the device was placed in the incubator at 37° C. and 5% CO₂.

Neurons seeded in the current technology where cultured with medium A and or with medium B. To maintain the cultures, every 2-3 days, 25 µl of medium from each well was discarded and 50 µl of the respective A or B preheated medium was added to the top wells, while 25 µl of medium was added to bottom wells. After 7 days in culture, the microfluidic assembly consisting of the multi-well grid layer (10) and microfluidic layer (20) was removed and discarded. All neurons remained attached to the upper surface (37) of the base layer (30), and were found to be organized in a precise pattern: with soma on the top side of the visualization window and axons elongating along over 100 parallel microchannels towards the lower side of each visualization window. In this example, each device comprised 24 visualization windows and each window has over 100 microchannels. Considering that more than one axon can grow on each microchannel, once can appreciate that the present technology enables imaging of over 2,400 axons/plate.

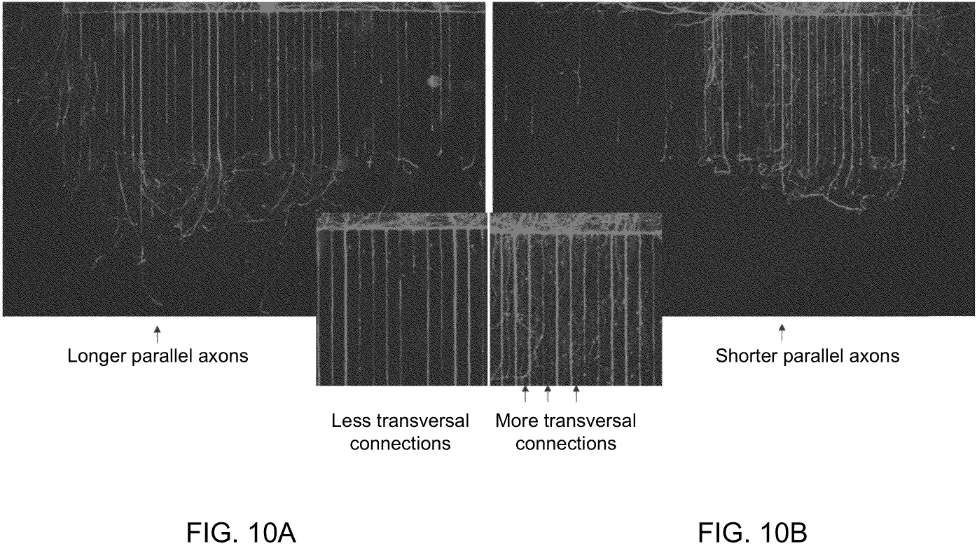

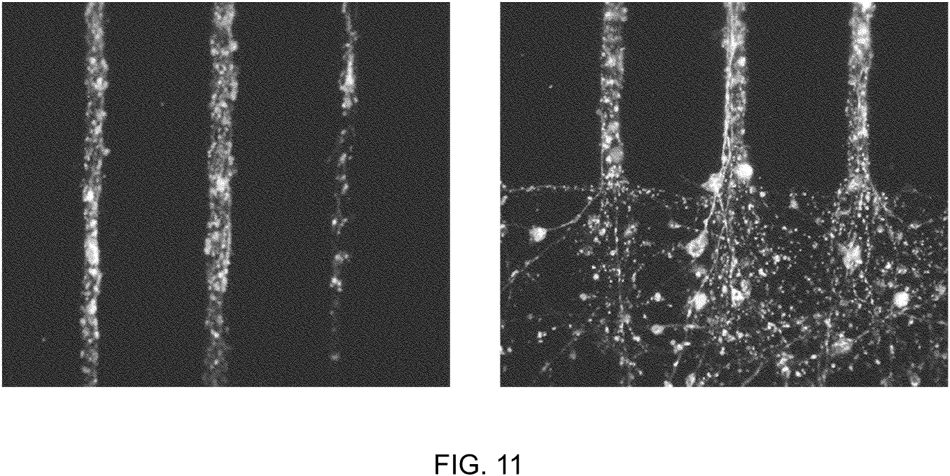

Neurons were maintained in culture in the respective medium A or B and after 14 days in culture, the neurons were fixed, stained with anti-TUBB3 rabbit antibody (1:1000) and AlexaTM Fluor-conjugated secondary antibodies (1:1000). Fluorescent images of the samples were acquired using an Axiovert 1 TM microscope (ZeissTM) with a 20× objective (Plan-Apochromat Pln ApoTM 20×/0.8; ZeissTM). As shown in FIG. 10, neurons cultured with medium (a) (FIG. 10A) produce axons 31% (15%) longer than neurons cultured with medium (b) (FIG. 10B). After removing the microfluidic layer and lifting the microchannels walls that separated the parallel axons, axons and dendrites that elongated in the microchannels are free to move, connect and form networks with axons and dendrites on neighbour channels. FIG. 10 is a representative image that clearly shows that there are connections between all channels in neurons cultured with medium (B) (FIG. 10B), while connections can only be seen between 24% of the microchannels in neurons cultured with medium (A) (FIG. 10A).

These results confirm that the present technology is advantageously compatible with HCA and HTS techniques and equipment enabling most of the process to be performed with multi-well pipettes, automated liquid handlers and automated microscope readers. The current technology thus enables for the first-time high capacity screening of axons (over 2,400 axons/plate). Moreover, the current technology enables high capacity screening of neuronal connections and circuitry formation: current technology first uses microfluidics as a mould to precisely position axons and dendrites, next, as we remove the microfluidics, neurons start to connect and the current technology enables live visualization of how the axons and dendrites connect, forming unique circuits when exposed to different molecules. Current technology enables for the first time the high capacity screening of compounds that affect network formation in vitro.

### Example 2: Assay to Model Neurological Diseases in Vitro

In neurodegenerative diseases such as Huntington’s disease (HD), Alzheimer’s disease and Parkinson’s disease, disease-specific proteins are expressed and accumulate. It is not clear which precise problems the accumulation of proteins incurs in patients suffering from these neurodegenerative proteopathies. Recent studies have shown that increased protein levels or changes in the ratios of different isoforms affect the movement of molecules along the axon, thereby disrupting neuronal function. Huntingtin, the protein involved in HD, plays a special role in axonal transport, and very recent studies have found that its activity - and the movement of its cargoes -are altered not only in HD but in other neurological diseases [Hélène Vitet, Vicky Brandt, Frédéric Saudou (2020) Traffic signaling: new functions of huntingtin and axonal transport in neurological disease. Curr Opin Neurobiol. May 11;63:122-130.] In the present example, it is shown how the present technology enables the screening in high capacity of axonal transport in dopaminergic neurons derived from induced pluripotent stem cells (iPSCs) from Parkinson Disease patients.

The upper surface (37) of the base layer (30) was coated with a solution of 100 µg/ml of PLO for 2 h at 37° C. and 5% CO2, followed by coating with laminin 10 µg/ml overnight at 4C. Next day, the upper surface was washed and assembled with (i) a microfluidic layer (20) comprising 24 microfluidic units (28) (each unit comprising a pair of inlets/outlets and one central chamber divided in two compartments by 120 microchannels) and (ii) a multi-well grid layer (10) consisting of a 384-well plate (e.g., as illustrated in FIG. 7).

Neurons derived from human iPSCs from Parkinson disease patients were acquired from The Neuro, McGill University Open iPSC biobank and resuspended in a 1 million cells/ml solution. Next, 10 µl of cell suspension (20,000 cells) were added to the top well on the right side of each microfluidic unit in the 384-well plate. For example, cells were added to wells A3, A7, A11, A15, A19, A23 and to wells of the same number on rows E, I and M. After incubation of the microplates for 20 min to promote cell adhesion to the upper surface (37) of the base layer (30), 50 µl of preheated cell medium was added to the top wells of each microfluidic unit and the system was placed in the incubator at 37° C. and 5% CO2.

Neurons seeded in the device of the technology where cultured for 4 weeks. To maintain the cultures, every 2-3 days, 25 µl of medium from each well was discarded and 50 µl of the respective A or B preheated medium was added to the top wells, while 25 µl of medium was added to bottom wells. After 4 weeks in culture, the neurons were fixed, stained with Tuj1, TOM20 and PDH antibodies (1:1000) and fluorescence-conjugated secondary antibodies (1:1000). Fluorescent images of the samples were acquired using an Axio ObserverTM Microscope (Zeiss) with a 40× objective (Plan-Apochromat Pln ApoTM 40x/0.8; Zeiss).

As shown in FIG. 11, the precise organization of neurons with soma on the top side of the visualization window and over 100 parallel axons elongating towards the lower side of each visualization window (each window comprising 100 microchannels). All axons are on the same plane, making the present technology compatible with automated imaging of a full plate in less than 2 h.

As it can appreciate, these results confirm that the current technology enables high content screening of over 2,400 axons/plate/2 h. The current technology enables for the first-time rapid high content screening of lead molecules directly on patient’s derived cells, providing a human model of Parkinson Disease in vitro, reducing animal experimentation and significantly accelerating drug development.

### Example 3: Assay to Model Neurodevelopmental Toxicity in Vitro

Astrocytes are characterized by their star-like shape astrocytes and they represent the most abundant cell type in the brain. Closely linked to neurons with pivotal roles in synaptic activity and blood-brain barrier function, the interaction between neurons and astrocytes is becoming increasingly important in neuroscience research [Role of glial cells in the formation and maintenance of synapses. Pfrieger et al., 2010]. Astrocytes support the metabolic and trophic development of neurons and serve a variety a well-established stage-specific functions in synaptogenesis, myelination and neuronal migration as they mature. In the past decade, it is becoming increasingly apparent that astrocytes are highly significant when there is disruption of the choreography of neural development, leading to disease pathogenesis in neurodevelopmental disorders such as autism [Astrocytes and disease: a neurodevelopmental perspective. Molofsky et al., 2012; Mechanisms of astrocyte development and their contributions to neurodevelopmental disorders. Sloan & Barres, 2014]. Understanding the impact of astrocyte dysfunction and behaviour, including the impact on surrounding neurons, is of high importance in understanding the underlying causes of such disorders and will pave the way towards future therapies. However, there are no efficient methods for HTS and HCA of co-cultures of neurons and astrocytes that enables evaluation of axonal health.

In this example, the current technology was used to grow co-cultures of astrocytes and cortical neurons derived from rats. The upper surface (37) of the base layer (30) was coated with a solution of 100 µg/ml of PDL for 2 h at 37° C. and 5% CO2. Next, the upper surface was washed and assembled with (i) a microfluidic layer (20) comprising microfluidic units (28) and (ii) a multi-well grid layer (10) consisting of a 384-well plate (e.g., as defined hereinbefore and illustrated in FIG. 7).

Animal procedures were performed in accordance with the Canadian Council on Animal Care Guidelines and were approved by the INRS Animal Care and Use Committee. Sprague Dawley rats were provided by Charles River Laboratories. The dissection of cortices of rat embryos at gestational date E19 was performed and astrocytes and neurons were collected and suspended in a solution with 1 million cells/ml. First, 2 µl of astrocyte suspension (2,000 cells) was added to the top and bottom wells on the right side of each the 24 microfluidic units in the 384-well plate. For example, cells were added to wells A3, A7, A11, A15, A19, A23 and to wells of the same number on rows C, E, G, I, J, M and O.

After incubation of the microplates for 20 min to promote cell adhesion to the upper surface (37) of the base layer (30), 50 µl of preheated cell medium was added to the top wells of each patter and the system was placed in the incubator at 37° C. and 5% CO2. Cells were kept in culture for 3 days.

After 3 days, cortical neurons were added to the plate. 10 µl of cell suspension (20,000 neurons) was added to the top well on the right side of each microfluidic unit. For example, cells were added to wells A3, A7, A11, A15, A19, A23 and to wells of the same number on rows E, I and M. After incubation of the microplates for 20 min to promote cell adhesion to the upper surface (37) of the base layer (30), 50 µl of preheated cell medium was added to the top wells of each patter and the system was placed in the incubator at 37° C. and 5% CO2.

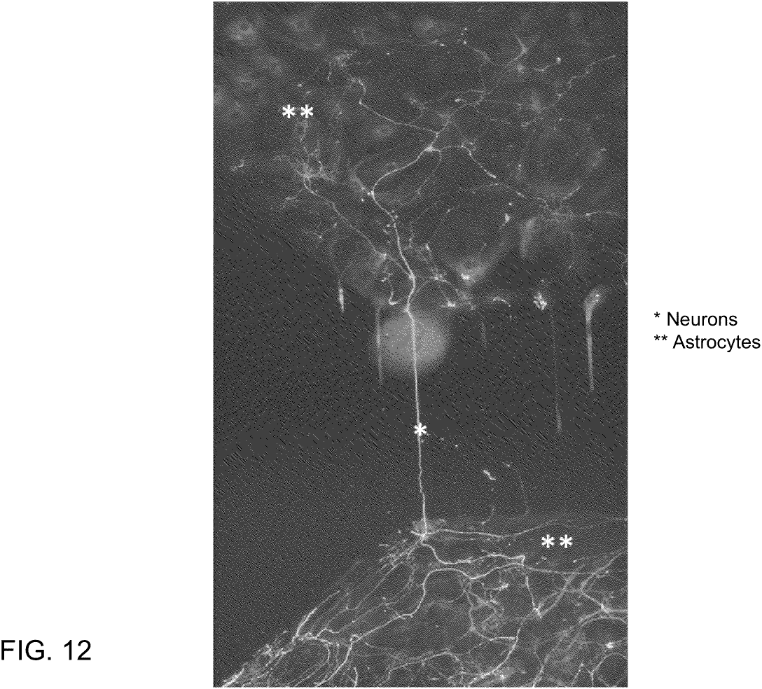

To maintain the cultures, every 2-3 days, 25 µl of medium from each well was discarded and 50 µl of preheated medium was added to the top wells, while 25 µl of medium was added to bottom wells. After 14 days in culture, neurons and astrocytes were fixed, stained with phalloidin (1:1000), anti-TUBB3 rabbit antibody (1:1000) and AlexaTM Fluor-conjugated secondary antibodies (1:1000). Fluorescent images of the samples were acquired using an Axiovert 1 TM Microscope (Zeiss) with a 20× objective (Plan-Apochromat Pln ApoTM 20×/0.8; Zeiss). As shown in FIG. 12, current technology enables efficient co-cultures of astrocytes and neurons where astrocytes form a uniform layer of cells on the upper and lower part of the visualization window, while neuronal cell bodies remained on the upper side of the visualization window with axons elongating vertically along over 100 parallel microchannels towards the lower side of each visualization window (each window comprising 100 microchannels). All astrocytes and axons are on the same plane, which makes their visualization compatible with automated imaging of a full plate is less than 2 h. The current technology thus enables for the first-time rapid high content screening of lead molecules on organized co-cultures of astrocytes and neurons, providing easy visualization of axonal health and significantly accelerating drug development.

Headings are included herein for reference and to aid in locating certain sections. These headings are not intended to limit the scope of the concepts described therein, and these concepts may have applicability in other sections throughout the entire specification. Thus, the present technology is not intended to be limited to the implementations shown herein but is to be accorded the widest scope consistent with the principles and novel features disclosed herein.

The singular forms “a”, “an” and “the” include corresponding plural references unless the context clearly dictates otherwise. Thus, for example, reference to “a well” includes one or more of such wells and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the implementations are approximations, the numerical values set forth in the specific examples are reported as precisely as possible. Any numerical value, however, inherently contains certain errors resulting from variations in experiments, testing measurements, statistical analyses and such.

Several alternative implementations and examples have been described and illustrated herein. The implementations of the technology described above are intended to be exemplary only. A person of ordinary skill in the art would appreciate the features of the individual implementations, and the possible combinations and variations of the components. A person of ordinary skill in the art would further appreciate that any of the implementations could be provided in any combination with the other implementations disclosed herein. It is understood that the technology may be embodied in other specific forms without departing from the central characteristics thereof. The present implementations and examples, therefore, are to be considered in all respects as illustrative and not restrictive, and the technology is not to be limited to the details given herein. Accordingly, while the specific implementations have been illustrated and described, numerous modifications come to mind.

1. A multi-well device for analysis of cells, comprising:
a multi-well grid layer comprising a plurality of wells;
a microfluidic layer comprising microchannels, wherein the microfluidic layer is configured for being positioned beneath the upper multi-well grid layer; and
a base layer configured for being positioned beneath the microfluidic layer and adapted for being detachably connected to the microfluidic layer and/or to the multi-well grid layer;
wherein once connected, the multi-well grid layer, the microfluidic layer and the base layer form at least one microchannel network enabling a fluid to flow therein from via the microchannels.

2. The multi-well device of claim 1, wherein the microfluidic layer comprises a microfluidic unit comprising a central main chamber with microchannels, at least one inlet, at least one outlet and arms extending from the main chamber to the at least one inlet and the at least one outlet, the arms providing a fluidic communication between the central main chamber, the at least one inlet and the at least one outlet.

3. The multi-well device of claim 2, wherein the at least one inlet, the at least one outlet, the central main chamber, the arms, and the microchannels are carved, printed, embossed, or moulded into the microfluidic layer.

4. The multi-well device of any one of claims 1 to 3, wherein the microfluidic layer comprises an upper surface that is bounded to a lower surface of the multi-well grid layer.

5. The multi-well device of any one of claims 1 to 4, wherein the multi-well grid layer comprises at least 6, 12, 24, 48, 96, 384, 1536 or 3456 wells.

6. The multi-well device of any one of claims 1 to 5, wherein at least one of the multi-well grid layers, the microfluidic layer and the base layer is made of glass and/or a polymeric material.

7. The multi-well device of any one of claims 1 to 6, wherein the base layer is made of an optically transparent material or a translucent material.

8. The multi-well device of claim 7, wherein the optically transparent material is selected from the group consisting of glass, acrylic, polystyrene (PS), cyclo-olefin-copolymer (COC), cycloolefin polymer (COP), a thermoplastic elastomer (TPE) and polydimethylsiloxane (PDMS).

9. The multi-well device of any one of claims 1 to 8, wherein the base layer is coated with a substance that promotes cellular adhesion, promotes cellular growth or repels cellular adhesion.

10. The multi-well device of any one of claims 1 to 9, wherein the base layer comprises connecting means for detachably connecting the base layer to the multi-well grid layer.

11. The multi-well device of any one of claims 1 to 10, wherein the base layer comprises a frame and a transparent layer bonded to the frame.

12. The multi-well device of any one of claims 1 to 10, wherein the base layer comprises a frame and a transparent layer integral to the frame.

13. The multi-well device of any one of claims 1 to 12, further comprising a lid adapted to be deposited over the multi-well grid layer.

14. The multi-well device of any one of claims 1 to 13, wherein the multi-well device is adapted for optical analysis of cells loaded into the at least one microchannel network.

15. The multi-well device of any one of claims 1 to 14, wherein the microfluidic layer is integral with the base layer.

16. The multi-well device of any one of claims 1 to 14, wherein the microfluidic layer is integral with the multi-well grid layer.

17. The multi-well device of any one of claims 1 to 16, wherein the microfluidic layer comprises a plurality of layers configured such that once superposed, the multi-well grid layer, the microfluidic layer, and the base layer form the least one microchannel network.

18. The multi-well device of claim 17, wherein at least one layer of the plurality of layers is integral with the base layer.

19. The multi-well device of claim 17 or 18, wherein at least one layer of the plurality of layers is integral with the multi-well grid layer.

20. A multi-well device for analysis of cells, comprising:
a multi-well grid layer comprising a plurality of wells;
a patterned layer configured for being positioned beneath the upper multi-well grid layer; and
a base layer configured for being positioned beneath the patterned layer and adapted for being detachably connected to the patterned layer and/or to the multi-well grid layer;
wherein once connected, the multi-well grid layer, the patterned layer and the base layer form at least one fluidic network enabling a fluid to flow therein.

21. The multi-well device of claim 20, wherein the patterned layer comprises a patterned unit.

22. The multi-well device of claim 21, wherein the patterned unit comprises a hole extending through a thickness of the patterned layer.

23. The multi-well device of claim 22, wherein the hole is an inlet configured to receive a fluid therein.

24. The multi-well device of claim 22, wherein the hole is an outlet configured for retrieving a fluid therefrom.

25. The multi-well device of claim 22, wherein the patterned unit comprises a plurality of holes extending through a thickness of the patterned layer, the plurality of holes comprising an inlet to receive a fluid therein and an outlet configured for retrieving a fluid therefrom.

26. The multi-well device of claim 25, wherein the inlet and the outlet are in fluid communication with a central main chamber.

27. The multi-well device of claim 26, wherein the inlet and the outlet are in fluid communication with the central main chamber via a corresponding arm.

28. The multi-well device of claim 26 or 27, wherein the patterned unit comprises an additional inlet and an additional outlet in fluid communication with the central main chamber.

29. The multi-well device of any one of claims 21 to 28, wherein the patterned unit is carved, printed, embossed, or moulded into the patterned layer.

30. The multi-well device of claim 21, wherein the patterned unit comprises a microfluidic unit.

31. The multi-well device of claim 30, wherein the microfluidic unit comprises a central main chamber with microchannels, and an inlet and an outlet both in fluid communication with the central main chamber.

32. The multi-well device of claim 31, wherein the inlet and the outlet are in fluid communication with the central main chamber via a corresponding arm.

33. The multi-well device of any one of claims 30 to 32, wherein the microfluidic unit is carved, printed, embossed, or moulded into the microfluidic layer.

34. The multi-well device of any one of claims 20 to 33, wherein the patterned layer comprises an upper surface that is bounded to a lower surface of the multi-well grid layer.

35. The multi-well device of any one of claims 22 to 34, wherein the multi-well grid layer comprises at least 6, 12, 24, 48, 96, 384, 1536 or 3456 wells.

36. The multi-well device of any one of claims 22 to 35, wherein at least one of the multi-well grid layers, the patterned layer and the base layer is made of glass and/or a polymeric material.

37. The multi-well device of any one of claims 22 to 36, wherein the base layer is made of an optically transparent material or a translucent material.

38. The multi-well device of claim 37, wherein the optically transparent material is selected from the group consisting of glass, acrylic, polystyrene (PS), cyclo-olefin-copolymer (COC), cycloolefin polymer (COP), a thermoplastic elastomer (TPE) and polydimethylsiloxane (PDMS).

39. The multi-well device of any one of claims 20 to 38, wherein the base layer is coated with a substance that promotes cellular adhesion, promotes cellular growth or repels cellular adhesion.

40. The multi-well device of any one of claims 22 to 39, wherein the base layer comprises connecting means for detachably connecting the base layer to the multi-well grid layer.

41. The multi-well device of any one of claims 22 to 40, wherein the base layer comprises a frame and a transparent layer bonded to the frame.

42. The multi-well device of any one of claims 22 to 40, wherein the base layer comprises a frame and a transparent layer integral to the frame.

43. The multi-well device of any one of claims 22 to 42, further comprising a lid adapted to be deposited over the multi-well grid layer.

44. The multi-well device of any one of claims 22 to 43, wherein the multi-well device is adapted for optical analysis of cells loaded into the at least one microchannel network.

45. The multi-well device of any one of claims 22 to 44, wherein the patterned layer is integral with the base layer.

46. The multi-well device of any one of claims 22 to 44, wherein the patterned layer is integral with the multi-well grid layer.

47. The multi-well device of any one of claims 22 to 44, wherein the patterned layer comprises a plurality of layers configured such that once superposed, the multi-well grid layer, the patterned layer, and the base layer form the least one microchannel network.

48. The multi-well device of claim 47, wherein at least one layer of the plurality of layers is integral with the base layer.

49. The multi-well device of claim 47 or 48, wherein at least one layer of the plurality of layers is integral with the multi-well grid layer.

50. A method for culturing cells, comprising:
providing a microfluidic assembly comprising:
a multi-well grid layer comprising a plurality of wells; and
a microfluidic layer comprising microchannels, the microfluidic layer being positionable beneath the upper multi-well grid layer;
providing a base layer positionable beneath the microfluidic layer and adapted for being detachably connected to the microfluidic layer and/or to the multi-well grid layer;
connecting the base layer to the microfluidic assembly, wherein once connected the multi-well grid layer, the microfluidic layer, the base layer form at least one microchannel network enabling a fluid to flow therein via the microchannels; and
loading cells to be cultured into at least one well of the plurality of wells.

51. The method of claim 50, further comprising analyzing the cells loaded into the at least one well.

52. The method of claim 50 or 51, the cells are cultured for a certain period of time prior to the analysis.

53. The method of claim 51 or 52, wherein analyzing the cells loaded into the at least one well comprises performing at least one of microscopy, electrical stimulation, absorbance, spectrophotometry, mass spectroscopy, or electrical impedance.

54. The method of any one of claims 50 to 53, wherein the at least one microchannel network comprises a central main chamber in fluid communication with at least one inlet and at least one outlet, and wherein the analysis of the cells loaded into the at least one well is carried out by analyzing cells that are in the central main chamber.

55. The method of any one of claims 50 to 54, wherein the multi-well grid layer comprises at least 6, 12, 24, 48, 96, 384, 1536 or 3456 wells.

56. The method of any one of claims 50 to 55, wherein the microfluidic assembly is adapted for high throughput optical analysis.

57. The method of any one of claims 50 to 56, further comprising, prior to loading the cells, coating the base layer with a substance that promotes cellular adhesion, promotes cellular growth or repels cellular adhesion.

58. The method of any one of claims 50 to 57, further comprising detaching the microfluidic assembly from the base layer, leaving organized cells attached to the base layer.

59. The method of any one of claims 50 to 58, wherein said method is for drug discovery, drug screening and/or systems biology.

60. A kit for analysis of cells, comprising:
a fluidic assembly comprising:
a multi-well grid layer comprising a plurality of wells; and
a patterned layer comprising microchannels and configured to be positioned beneath the upper multi-well grid layer; and
a base layer configured for being positioned beneath the patterned layer and adapted for being detachably connected to the patterned layer and/or to the multi-well grid layer;
wherein once connected, the multi-well grid layer, the microfluidic layer, and the base layer form at least one microchannel network enabling a fluid to flow therein via the microchannels.

61. The kit of claim 60, further comprising at least one feature as described in any one of claims 21 to 49.

62. A multi-well device for analysis of cells comprising:
a microfluidic layer comprising:
a central main chamber comprising a first compartment and a second compartment, wherein the first and second compartments are separated by a plurality of microfluidic channels, the microfluidic channels providing a fluidic communication between the first and second compartments;
a first inlet and a first outlet disposed at opposite ends of the first compartment of the central main chamber;
two arms extending diagonally in opposite directions from the first compartment of the central main chamber, the arms providing a fluidic communication of the first inlet and the first outlet with the first compartment of the central main chamber;
a second inlet and a second outlet disposed at opposite ends of the second compartment of the central main chamber;
two arms extending diagonally in opposite directions from the second compartment of the central main chamber, the arms providing a fluidic communication of the second inlet and the second outlet with the second compartment of the central main chamber;
a multi-well grid layer configured to be superposed over the microfluidic layer;
the multi-well grid layer comprising a corresponding well extending therethrough and vertically aligned with the central main chamber, the first inlet, the first outlet, the second inlet and the second outlet of the microfluidic layer, respectively;
a base layer configured for being positioned beneath the microfluidic layer.

63. The multi-well device of claim 62, wherein the multi-well grid layer comprises at least nine (9) wells that are distributed in a 3 × 3 configuration, and wherein said 3 × 3 configuration comprises (i) a center well vertically aligned over the main chamber and (ii) four opposite corner wells vertically aligned over the first inlet, the first outlet, the second inlet and the second outlet of the microfluidic layer, respectively.

64. The multi-well device of claim 62 or 63, wherein the central main chamber, the first inlet, the first outlet, the second inlet and the second outlet of the microfluidic layer forms together a single microfluidic unit having an X-configuration.

65. The multi-well device of any one of claims 62 to 64, wherein the base layer is detachably connected to the microfluidic layer and/or to the multi-well grid layer.

66. The multi-well device of any one of claims 62 to 65, wherein the microfluidic layer comprises an upper surface that is bounded to a lower surface of the multi-well grid layer.

67. The multi-well device of any one of claims 62 to 66, wherein the multi-well grid layer comprises at least 6, 12, 24, 48, 96, 384, 1536 or 3456 wells.

68. The multi-well device of any one of claims 62 to 67, wherein at least one of the multi-well grid layer, the microfluidic layer and the base layer is made of glass and/or a polymeric material.

69. The multi-well device of any one of claims 62 to 68, wherein the base layer is transparent or translucid.

70. A device for analysis of cells, comprising:
a microfluidic layer configured for being placed in contact with an electrode layer comprising electrodes, the microfluidic layer comprising a microfluidic unit having microchannels configured to receive at least a component of the cells therein, the microchannels being provided in a spaced-apart relationship relative to each other; and
an alignment feature for aligning the microchannels of the microfluidic unit with the electrodes of the electrode layer once the microfluidic layer is placed in contact with the electrode layer to achieve a predetermined organized architecture of the microchannels relative to the electrodes.

71. The device of claim 70, wherein each of the electrodes comprises an electrode tip, and the alignment feature enables alignment of at least one microchannel with a predetermined number of the electrode tips.

72. The device of claim 70, wherein each of the electrodes comprises an electrode tip, and the alignment feature enables alignment of a predetermined number of the electrode tips laterally along the microchannels.

73. The device of claim 70, wherein each of the electrodes comprises an electrode tip, and the alignment feature enables positioning of a predetermined number of the microchannels over a predetermined number of electrode tips.

74. The device of claim 70, wherein each of the electrodes comprises an electrode tip, and the alignment feature enables placement of the microfluidic layer over the electrode layer such that the microchannels extend substantially vertically or substantially horizontally and intersect a predetermined number of electrode tips.

75. The device of claim 70, wherein each of the electrodes comprises an electrode tip, and the alignment feature enables positioning the microfluidic layer such that the microchannels intersect the electrode tips along an entire diameter of the electrode tips.

76. The device of any one of claims 70 to 75, wherein the microfluidic layer comprises a plurality of microfluidic units, each microfluidic unit of the plurality of microfluidic units being associated with a corresponding electrode grid of the electrode layer.

77. The device of claim 76, wherein the alignment feature comprises a microfluidic layer alignment opening defined in the microfluidic layer, the microfluidic layer alignment opening being engageable with an electrode layer protruding member extending upwardly from the electrode layer.

78. The device of claim 76, wherein the alignment feature comprises a microfluidic layer protruding member extending downwardly from the microfluidic layer, the microfluidic layer protruding member being engageable with an electrode layer alignment cavity defined in the electrode layer.

79. The device of claim 76, wherein the alignment feature comprises a series of ridges protruding from a lower surface of the microfluidic layer, the series of ridges being engageable with a complimentary series of furrows defined in an upper surface of the electrode layer.

80. The device of claim 76, wherein the alignment feature comprises an alignment marking provided on the microfluidic layer, the alignment marking having a predetermined configuration based on a distribution of the electrodes of the electrode grid to enable alignment of the microchannels with the electrodes.

81. The device of any one of claims 76 to 80, wherein the alignment feature comprises a microfluidic layer alignment frame coupled to the microfluidic layer, the microfluidic layer alignment frame being configured to engage with an electrode layer frame to secure the microfluidic layer in the predetermined organized architecture of the microchannels relative to the electrodes.

82. The device of claim 81, wherein the microfluidic layer alignment frame is engageable with the electrode layer frame via a snap-on mechanism.

83. The device of any one of claims 70 to 75, wherein the microfluidic layer comprises a single microfluidic unit, the single microfluidic unit being associated with a corresponding electrode grid of the electrode layer.

84. The device of claim 83, wherein the alignment feature comprises a microfluidic layer alignment opening defined in the microfluidic layer, the microfluidic layer alignment opening being engageable with an electrode layer protruding member extending upwardly from the electrode layer.

85. The device of claim 83, wherein the alignment feature comprises a microfluidic layer protruding member extending downwardly from the microfluidic layer, the microfluidic layer protruding member being engageable with an electrode layer alignment cavity defined in the electrode layer.

86. The device of claim 83, wherein the alignment feature comprises a series of ridges protruding from a lower surface of the microfluidic layer, the series of ridges being engageable with a complimentary series of furrows defined in an upper surface of the electrode layer.

87. The device of claim 83, wherein the alignment feature comprises an alignment marking provided on the microfluidic layer, the alignment marking having a predetermined configuration based on a distribution of the electrodes of the electrode grid to enable alignment of the microchannels with the electrodes.

88. The device of any one of claims 83 to 87, wherein the alignment feature comprises a microfluidic layer alignment frame coupled to the microfluidic layer.

89. The device of claim 88, wherein the microfluidic layer alignment frame is configured to engage with a peripheral wall of a well having the electrode layer as a bottom wall.

90. The device of claim 89, wherein the microfluidic layer alignment frame comprises an alignment tab configured to be received within an alignment tab-receiving cavity defined in the peripheral wall of the well.

91. The device of claim 89, wherein the microfluidic layer alignment frame comprises a predetermined number of alignment tabs configured to be received in a corresponding predetermined number of alignment tab-receiving cavities defined in the peripheral wall of the well.

92. The device of any one of claims 88 to 91, wherein the microfluidic layer alignment frame extends upwardly from the microfluidic layer, and the alignment tabs are provided in an upper portion of the microfluidic layer alignment frame.

93. The device of any one of claims 88 to 91, wherein the microfluidic layer alignment frame at least partially surrounds an outer periphery of the microfluidic layer.

94. The device of any one of claims 88 to 93, wherein the microfluidic layer alignment frame is engageable with an electrode layer frame via a snap-on mechanism.

95. The device of any one of claims 70 to 94, wherein the at least a component of the cells received in the microchannels comprises axons of neuronal cells.

96. A device for establishing electrical communication with cells, comprising:
an electrode layer configured for being placed in contact with a microfluidic layer comprising a microfluidic unit having microchannels configured to receive at least a component of the cells therein, the electrode layer comprising electrodes for interacting with at least a component of cells received in the microchannels; and
an alignment feature for aligning the electrodes with the microchannels of the microfluidic unit once the electrode layer is placed in contact with the microfluidic layer to achieve a predetermined organized architecture of the microchannels relative to the electrodes.

97. The device of claim 96, wherein each of the electrodes comprises an electrode tip, and the alignment feature enables alignment of a predetermined number of the electrode tips with at least one microchannel.

98. The device of claim 96, wherein each of the electrodes comprises an electrode tip, and the alignment feature enables alignment of a predetermined number of the electrode tips laterally along the microchannels.

99. The device of claim 96, wherein each of the electrodes comprises an electrode tip, and the alignment feature enables positioning of a predetermined number of electrodes tips over a predetermined number of microchannels.

100. The device of claim 96, wherein each of the electrodes comprises an electrode tip, and the alignment feature enables placement of the electrode layer in contact with the microfluidic layer such that a predetermined number of the electrode tips intersect the microchannels.

101. The device of claim 96, wherein each of the electrodes comprises an electrode tip, and the alignment feature enables positioning the electrode layer such that the microchannels intersect the electrode tips along an entire diameter of the electrode tips.

102. The device of any one of claims 96 to 101, wherein the electrodes of the electrode layer are provided as a plurality of electrode grids that are placeable in contact with a corresponding microfluidic unit of the microfluidic layer.

103. The device of claim 102, wherein the alignment feature comprises an electrode layer protruding member extending upwardly from the electrode layer, the electrode layer protruding member being engageable with a microfluidic layer alignment opening defined in the microfluidic layer.

104. The device of claim 102, wherein the alignment feature comprises an electrode layer alignment cavity defined in the electrode layer, the electrode layer alignment cavity being engageable with a microfluidic layer protruding member extending downwardly from the microfluidic layer.

105. The device of claim 102, wherein the alignment feature comprises a series of ridges protruding from an upper surface of the electrode layer, the series of ridges being engageable with a complimentary series of furrows defined in a lower surface of the microfluidic layer.

106. The device of claim 102, wherein the alignment feature comprises an electrode layer alignment frame surrounding the electrode layer, the electrode layer alignment frame being configured to engage with a microfluidic layer alignment frame to secure the electrode layer in the predetermined organized architecture of the microchannels relative to the electrodes.

107. The device of claim 106, wherein the electrode layer alignment frame is engageable with the microfluidic layer alignment frame via a snap-on mechanism.

108. The device of any one of claims 96 to 101, wherein the electrode layer is provided as a bottom wall of a well of a multi-well plate.

109. The device of claim 108, wherein the electrodes of the electrode layer are provided as an electrode grid.

110. The device of claim 108 or 109, wherein the alignment feature comprises an electrode layer protruding member extending upwardly from the electrode layer, the electrode layer protruding member being engageable with a microfluidic layer alignment opening defined in the microfluidic layer.

111. The device of claim 108 or 109, wherein the alignment feature comprises an electrode layer alignment cavity defined in the electrode layer, the electrode layer alignment cavity being engageable with a microfluidic layer protruding member extending downwardly from the microfluidic layer.

112. The device of claim 108 or 109, wherein the alignment feature comprises a series of ridges protruding from an upper surface of the electrode layer, the series of ridges being engageable with a complimentary series of furrows defined in a lower surface of the microfluidic layer.

113. The device of claim 108 or 109, wherein the alignment feature comprises an alignment tab-receiving cavity defined in a peripheral wall of the well, the alignment tab-receiving cavity being configured to receive therein an alignment tab extending from a microfluidic layer alignment frame coupled to the microfluidic layer.

114. The device of claim 108 or 109, wherein the peripheral wall of the well comprises a predetermined number of alignment tab-receiving cavities for receiving a corresponding predetermined number of alignment tabs tab extending from a microfluidic layer alignment frame coupled to the microfluidic layer.

115. The device of claim 113 or 114, wherein the alignment tab-receiving cavity is provided in an upper portion of the well.

116. The device of any one of claims 96 to 115, wherein the at least a component of the cells received in the microchannels comprises axons of neuronal cells.

117. A device for analysis of cells, comprising:
an electrode layer comprising electrodes for establishing electrical communication with the cells;
a microfluidic layer configured for being placed in contact with the electrode layer, the microfluidic layer comprising a microfluidic unit having microchannels configured to receive at least a component of the cells therein, the microchannels being provided in a spaced-apart relationship relative to each other; and
an alignment feature for aligning the microchannels of the microfluidic unit with the electrodes of the electrode layer once the microfluidic layer is placed in contact with the electrode layer to achieve a predetermined organized architecture of the microchannels relative to the electrodes.

118. The device of claim 117, comprising one or more features as defined in any one of claims 71 to 95 or any one of claims 97 to 116.

119. A method for placing a microfluidic layer in contact with an electrode layer, the method comprising:
placing the microfluidic layer in proximity of the electrode layer; and aligning microchannels of a microfluidic unit of the microfluidic layer with electrodes of the electrode layer using an alignment feature to achieve a predetermined organized architecture of the microchannels relative to the electrodes, the microchannels being configured to receive at least a component of cells therein and being provided in a spaced-apart relationship relative to each other.

120. The method of claim 119, wherein each of the electrodes comprises an electrode tip, and aligning the microchannels of the microfluidic unit with the electrodes of the electrode layer using the alignment feature comprises aligning at least one microchannel with a predetermined number of the electrode tips.

121. The method of claim 119, wherein each of the electrodes comprises an electrode tip, and aligning the microchannels of the microfluidic unit with the electrodes of the electrode layer using the alignment feature comprises aligning a predetermined number of the electrode tips laterally along the microchannels.

122. The method of claim 119, wherein each of the electrodes comprises an electrode tip, and aligning the microchannels of the microfluidic unit with the electrodes of the electrode layer using the alignment feature comprises positioning a predetermined number of the microchannels over a predetermined number of electrode tips.

123. The method of claim 119, wherein each of the electrodes comprises an electrode tip, and aligning the microchannels of the microfluidic unit with the electrodes of the electrode layer using the alignment feature comprises placing the microfluidic layer over the electrode layer such that the microchannels extend substantially vertically or substantially horizontally and intersect a predetermined number of electrode tips.

124. The method of claim 119, wherein each of the electrodes comprises an electrode tip, and aligning the microchannels of the microfluidic unit with the electrodes of the electrode layer using the alignment feature comprises positioning the microfluidic layer such that the microchannels intersect the electrode tips along an entire diameter of the electrode tips.

125. The method of any one of claims 119 to 124, wherein aligning the microchannels of the microfluidic unit with the electrodes of the electrode layer using the alignment feature comprises engaging a microfluidic layer alignment opening defined in the microfluidic layer with an electrode layer protruding member extending upwardly from the electrode layer.

126. The method of any one of claims 119 to 124, wherein aligning the microchannels of the microfluidic unit with the electrodes of the electrode layer using the alignment feature comprises engaging a microfluidic layer protruding member extending downwardly from the microfluidic layer with an electrode layer alignment cavity defined in the electrode layer.

127. The method of any one of claims 119 to 124, wherein aligning the microchannels of the microfluidic unit with the electrodes of the electrode layer using the alignment feature comprises engaging a series of ridges protruding from a lower surface of the microfluidic layer with a complimentary series of furrows defined in an upper surface of the electrode layer.

128. The method of any one of claims 119 to 124, wherein aligning the microchannels of the microfluidic unit with the electrodes of the electrode layer using the alignment feature comprises aligning an alignment marking provided on the microfluidic layer with the electrodes, the alignment marking having a predetermined configuration based on a distribution of the electrodes.

129. The method of any one of claims 119 to 128, wherein aligning the microchannels of the microfluidic unit with the electrodes of the electrode layer using the alignment feature comprises engaging an alignment tab of a microfluidic layer alignment frame coupled to the microfluidic layer with an alignment tab-receiving cavity defined in a peripheral wall of a well having the electrode layer as a bottom wall.

130. The method of any one of claims 119 to 128, wherein aligning the microchannels of the microfluidic unit with the electrodes of the electrode layer using the alignment feature comprises engaging a microfluidic layer alignment frame coupled to the microfluidic layer with an electrode layer frame surrounding the electrode layer.

131. The method of claims 130, wherein the microfluidic alignment frame is engageable with the electrode layer frame via a snap-on mechanism.

132. The method of any one of claims 119 to 131, wherein the at least a component of the cells received in the microchannels comprises axons of neuronal cells.

133. A multi-well device for analysis of cells, comprising:
a multi-well grid layer comprising a plurality of wells;
a microfluidic layer comprising a microfluidic unit having microchannels configured to receive at least a component of the cells therein, the microchannels being provided in a spaced-apart relationship relative to each other; and
an electrode layer comprising electrodes placeable in contact with the microfluidic layer to achieve a predetermined organized architecture of the microchannels relative to the electrodes.

134. The multi-well device of claim 133, further comprising a base layer positionable underneath the electrode layer, the base layer being detachably connectable to at least one of the microfluidic layer, the electrode layer or the multi-well grid layer.

135. The multi-well device of claim 133, further comprising a base layer positionable underneath the microfluidic layer, the electrode layer being integrated into the base layer.

136. The multi-well device of any one of claims 133 to 135, further comprising one or more features as defined in any one of claims 71 to 95 or any one of claims 97 to 116.

137. A device for analysis of cells, comprising:
a plurality of microfluidic layers each comprising a microfluidic unit having microchannels configured to receive at least a component of the cells therein; and
a microfluidic layer engaging frame engageable with the plurality of microfluidic layers such that once engaged, the microfluidic layer engaging frame and the plurality of microfluidic layers form a unitary structure, the unitary structure being engageable with a multi-well plate comprising a plurality of wells each comprising an electrode layer having electrodes and each being configured for receiving therein a corresponding one of the plurality of microfluidic layers to place the corresponding one of the plurality of microfluidic layers in contact with the electrode layer to achieve a predetermined organized architecture of the microchannels relative to the electrodes.

138. The device of claim 137, wherein the microfluidic layer engaging frame comprises a base wall comprising microfluidic layer openings defined therethrough to enable fluid communication with the microfluidic unit and insertion and/or removal of fluids into the microfluidic unit.

139. The device of claim 137 or 138, wherein the microfluidic layer engaging frame comprises a multi-well plate alignment feature extending downwardly toward the multi-well plate, the multi-well plate alignment feature being insertable into an alignment feature receiving opening defined in the multi-well plate to align the microfluidic layer engaging frame with the multi-well plate.

140. The device of any one of claims 137 to 139, wherein the microfluidic layer engaging frame comprises an engagement feature engageable with an engagement feature connector of the multi-well plate.

141. The device of claim 140, wherein the engagement feature is engageable with the engagement feature connector of the multi-well plate via a snap-on mechanism.

142. The device of any one of claims 137 to 141, wherein the plurality of microfluidic layers is integral with the microfluidic layer engaging frame.

143. The device of any one of claims 137 to 142, wherein the microfluidic layer engaging frame comprises a microfluidic layer alignment feature configured for placement of the corresponding one of the plurality of microfluidic layers at a given location of the microfluidic layer engaging frame.

144. A device for analysis of cells, comprising:
a microfluidic layer comprising a plurality of microfluidic units each comprising microchannels; and
a multi-well grid layer comprising a plurality of bottomless wells, the multi-well grid layer being positionable over the microfluidic layer; and
a well identification feature provided on an upper surface of the multi-grid layer, the well identification feature being associated with a corresponding microfluidic unit of the plurality of microfluidic units to enable visual identification of at least one predetermined well of the multi-well grid layer that is in fluid communication with a component of the corresponding microfluidic unit.

145. The device of claim 144, wherein each microfluidic unit comprises:
first and second inlets;
first and second outlets, the first outlet being in fluid communication with the first inlet via a first compartment and the second outlet being fluid communication with the second inlet via a second compartment;
wherein the microchannels extend between the first and second compartments.

146. The device of claim 145, wherein the well identification feature comprises a well marking.

147. The device of claim 146, wherein the well marking comprises an individual well marking associated with each one of the first and second inlets and the first and second outlets once the multi-well grid layer is positioned over the microfluidic layer, each one of the first and second inlets and the first and second outlets corresponding to a respective component of the microfluidic layer.

148. The device of claim 146 or 147, wherein the at least one predetermined well of the multi-well grid layer comprises a plurality of wells associated with the corresponding microfluidic unit, and the well marking comprises an outer well marking provided at an outer periphery of the plurality of wells of the multi-well grid layer to visually identify the corresponding microfluidic unit once the multi-well grid layer is positioned over the microfluidic layer.

149. The device of claim 144, wherein the well identification feature comprises a well identification layer superposable to an upper surface of the multi-well grid layer.

150. The device of claim 149, wherein the well identification layer comprises columns provided in between longitudinally spaced-apart microfluidic units of the plurality of microfluidic units.

151. The device of claim 149 or 150, wherein the well identification layer comprises rows provided in between laterally spaced-apart microfluidic units of the plurality of microfluidic units.

152. A method for culturing cells, comprising:
providing a fluidic assembly comprising:
a multi-well grid layer comprising a plurality of wells; and
a patterned layer being positionable beneath the upper multi-well grid layer;
providing a base layer positionable beneath the patterned layer and adapted for being detachably connected to the patterned layer and/or to the multi-well grid layer;
connecting the base layer to the fluidic assembly, wherein once connected the multi-well grid layer, the patterned layer, the base layer form at least one fluidic network enabling a fluid to flow therein; and
loading cells to be cultured into at least one well of the plurality of wells.
